(19) United States
(12) Patent Application Publication
(10) Pub. No.: US 2024/0301380 A1
(43) Pub. Date: Sep. 12, 2024

(54) Compositions and Methods for Treating Fabry Disease

(71) Applicant: Spark Therapeutics, Inc., Philadelphia, PA (US)

(72) Inventors: Sean Armour, Holland, PA (US); Daniel Cohen, Philadelphia, PA (US); Christopher Riling, Exton, PA (US)

(21) Appl. No.: 18/261,285

(22) PCT Filed: Jan. 13, 2022
(86) PCT No.: PCT/US2022/070184

§ 371 Date: Jul. 13, 2023

Related U.S. Application Data
(60) Provisional application No. 63/137,235, filed on Jan. 14, 2021.
(60) Provisional application No. 63/264,356, filed on Nov. 19, 2021.

(51) Int. Cl. C12N 9/40; A61K 48/00; A61P 3/00; C12N 15/86

(52) U.S. Cl. CPC C12N 9/2465; A61K 48/0058; A61P 3/00; C12N 15/86; C07K 2319/02; C12N 2750/14143; C12N 2830/008; C12N 2830/15; C12N 2830/42; C12N 2830/50; C12Y 302/01022

(57) ABSTRACT

Polynucleotides including nucleic acids encoding α-galactosidase A (GLA) are described. Also described are expression cassettes, vectors, cells, and cell lines containing the polynucleotides, as well as methods of using the polynucleotides to treat lysosomal storage disorders such as Fabry disease.

### RELATED APPLICATIONS

This application claims priority to U.S. Provisional Patent Application No. 63/137,235 filed Jan. 14, 2021 and U.S. Provisional Patent Application No. 63/264,356 filed Nov. 19, 2021. The entire contents of the foregoing applications are incorporated herein by reference, including all text, tables, sequence listings and drawings.

### REFERENCE TO SEQUENCE LISTING SUBMITTED ELECTRONICALLY

This application contains a sequence listing, which is submitted electronically via EFS-Web as an ASCII formatted sequence listing with a file name “SequenceListing4WO”, creation date of Jan. 13, 2022 and having a size of 281 KB. The sequence listing submitted via EFS-Web is part of the specification and is herein incorporated by reference in its entirety.

### FIELD OF THE INVENTION

The invention relates to the field of gene therapy. In particular, it relates to optimized cassettes for expression of human α-galactosidase A and methods of using the same for treating lysosomal storage diseases, in particular Fabry disease.

### BACKGROUND OF THE INVENTION

Fabry disease is an X-linked lysosomal storage disease, with an estimated prevalence of approximately 1:40,000. Fabry disease is caused by a deficiency in the lysosomal enzyme α-galactosidase A (GLA; α-gal A). The enzyme deficiency leads to the buildup of glycosphingolipid globotriaosylceramide (GL3 or GL-3 or Gb3) and globotriaosylsphingosine (lyso-GL3 or lyso-GL-3 or lyso-Gb3), resulting in progressive kidney disease, peripheral neuropathy, early-onset cerebrovascular disease, gastrointestinal symptoms, hypertrophic cardiomyopathy, arrhythmias, corneal whorls, and angiokeratomas. The average lifespan of a Fabry patient not treated with enzyme replacement therapy, from renal, cardiac, and/or cerebral complications from vascular disease, is 50 years for men and 70 years for women (Lidove et al., Int. J Clin. Pract. 2007; 61:293-302).

Enzyme replacement therapy (ERT) is available for Fabry disease, but it does not represent a cure, requiring weekly intravenous administration for the lifetime of the patients. Additionally, a significant proportion of patients develop neutralizing antibodies (NAb) to the α-galactosidase, thus rendering ERT ineffective (Linthorst et al., Kidney Int. 2004; 66(4):1589-1595).

### BRIEF SUMMARY OF THE INVENTION

Disclosed herein are optimized cassettes for liver-directed expression of a secretable version of human α-galactosidase A (GLA). These optimizations to the cassettes lead to an increase in GLA secretion from liver and enable hepatic gene transfer to achieve circulating levels of GLA sufficient to cross-correct GLA deficiency systemically in subjects. These cassettes will be useful as a gene therapy treatment of subjects with Fabry disease and other diseases and disorders treatable with GLA.

In one general aspect, the invention relates to a polynucleotide comprising a nucleic acid encoding α-galactosidase A (GLA), wherein the nucleic acid is selected from the group consisting of: (1) a polynucleotide having at least 75% sequence identity to the sequence of SEQ ID NO: 15, (2) a polynucleotide having at least 84% sequence identity to the sequence of SEQ ID NO: 16, (3) a polynucleotide having at least 86% sequence identity to the sequence of SEQ ID NO: 17, (4) a polynucleotide having at least 86% sequence identity to the sequence of SEQ ID NO: 18, and (5) a polynucleotide having at least 83% sequence identity to the sequence of SEQ ID NO: 19, optionally, the GLA comprises the amino acid sequence of SEQ ID NO: 100.

In certain embodiments, the nucleic acid contains fewer than 14 CpG dinucleotides, optionally 0 CpG dinucleotides.

In certain embodiments, the nucleic acid encoding GLA has a sequence of any one of SEQ ID NOs: 15-19.

In certain embodiments, the nucleic acid encoding GLA further comprises one or more introns positioned anywhere within the nucleic acid encoding the GLA. In certain embodiments, an intron is positioned between nucleotides 78 and 79 of the nucleic acid encoding the GLA, wherein the nucleotide positions are given in reference to the coding sequence of GLA having a sequence of SEQ ID NO: 14.

In certain embodiments, the intron is selected from the group consisting of introns from vitronectin1 (VTN1) gene, retinol binding protein 4 (RBP4) gene, mouse IgG heavy chain A (IgHA) gene, and mouse IgG heavy chain μ (IgHμ) gene. In certain embodiments, the one or more introns are selected from the sequences of SEQ ID NOs: 49-52.

In certain embodiments, the nucleic acid encoding GLA has a sequence of any one of SEQ ID NOs: 43-46.

In certain embodiments, the GLA comprises the amino acid sequence of SEQ ID NO: 100 with one amino acid substitution selected from the group consisting of Gln57Lys, Gln111Glu, Lys213Glu, Lys237Gln, Phe248Thr, Gly334Glu, and Gly346Asn.

In certain embodiments, the GLA comprises the amino acid sequence of SEQ ID NO: 100 with any two amino acid substitutions selected from the group consisting of Gln57Lys, Gln111Glu, Lys213Glu, Lys237Gln, Phe248Thr, Gly334Glu, and Gly346Asn.

In certain embodiments, the GLA comprises the amino acid sequence of SEQ ID NO: 100 with any three amino acid substitutions selected from the group consisting of Gln57Lys, Gln111Glu, Lys213Glu, Lys237Gln, Phe248Thr, Gly334Glu, and Gly346Asn.

In certain embodiments, the GLA comprises the amino acid sequence of SEQ ID NO: 100 with any four amino acid substitutions selected from the group consisting of Gln57Lys, Gln111Glu, Lys213Glu, Lys237Gln, Phe248Thr, Gly334Glu, and Gly346Asn.

In certain embodiments, the GLA comprises the amino acid sequence of SEQ ID NO: 100 with any five amino acid substitutions selected from the group consisting of Gln57Lys, Gln111Glu, Lys213Glu, Lys237Gln, Phe248Thr, Gly334Glu, and Gly346Asn.

In certain embodiments, the GLA comprises the amino acid sequence of SEQ ID NO: 100 with any six amino acid substitutions selected from the group consisting of Gln57Lys, Gln111Glu, Lys213Glu, Lys237Gln, Phe248Thr, Gly334Glu, and Gly346Asn.

In certain embodiments, the GLA comprises the amino acid sequence of SEQ ID NO: 100 with the seven amino acid substitutions of Gln57Lys, Gln111Glu, Lys213Glu, Lys237Gln, Phe248Thr, Gly334Glu, and Gly346Asn.

In certain embodiments, the GLA comprises the amino acid sequence of SEQ ID NO: 48.

In certain embodiments, the nucleic acid encoding GLA comprises the coding sequence of SEQ ID NO: 47.

In certain embodiments, the polynucleotide further comprises a second nucleic acid encoding a signal peptide sequence positioned at the 5′ end of the nucleic acid encoding the GLA.

In certain embodiments, the signal peptide sequence is a heterologous signal peptide sequence.

In certain embodiments, the signal peptide sequence is an endogenous or native GLA signal peptide sequence.

In certain embodiments, the signal peptide is selected from the group consisting of human chymotrypsinogen B2 signal peptide, AHSG signal peptide, CD300 signal peptide, LAMP1 signal peptide, Notch 2 signal peptide, ORM1 signal peptide, TF signal peptide, and native GLA signal peptide, or a variant thereof.

In certain embodiments, the signal peptide is a human chymotrypsinogen B2 signal peptide, optionally a human chymotrypsinogen B2 signal peptide having an amino acid sequence of SEQ ID NO: 41, or a variant thereof.

In certain embodiments, the signal peptide is a human chymotrypsinogen B2 signal peptide, optionally a human chymotrypsinogen B2 signal peptide having a coding sequence of any one of SEQ ID NOs: 1-5.

In certain embodiments, the polynucleotide encodes a precursor GLA having a sequence of any one of SEQ ID NOs: 101-109.

In certain embodiments, the polynucleotide comprises a sequence of any one of SEQ ID NOs: 64-81.

In certain embodiments, the invention relates to an expression cassette comprising the polynucleotide comprising the nucleic acid encoding GLA operably linked to an expression control element.

In certain embodiments, the invention relates to an expression cassette comprising the polynucleotide comprising the nucleic acid encoding human GLA, operably linked to an expression control element.

In certain embodiments, the expression control element is a liver-specific expression control element.

In certain embodiments, the expression control element of the expression cassette is positioned 5′ of the polynucleotide, wherein the expression control element optionally comprises an ApoE/hAAT enhancer/promoter sequence.

In certain embodiments, the expression cassette further comprises a poly-adenylation sequence positioned 3′ of the polynucleotide, wherein the poly-adenylation sequence optionally comprises a bovine growth hormone (bGH) polyadenylation sequence.

In certain embodiments, the expression control element or poly-adenylation sequence of the expression cassette is CpG-reduced compared to the wild-type expression control element or polyadenylation sequence.

In certain embodiments, the expression cassette further comprises an intron positioned between the 3′ end of the expression control element and the 5′ end of the polynucleotide, wherein the intron optionally comprises an hBB2m1 intron.

In certain embodiments AAV ITR(s) flank the 5′ and/or 3′ terminus of the polynucleotide or the expression cassette.

In certain embodiments, the invention relates to an adeno-associated virus (AAV) vector comprising the polynucleotide or expression cassette.

In certain embodiments, the AAV vector comprises: (a) one or more of an AAV capsid, and (b) one or more AAV inverted terminal repeats (ITRs), wherein the AAV ITR(s) flanks the 5′ or 3′ terminus of the polynucleotide or the expression cassette.

In certain embodiments, at least one or more of the ITRs of the AAV vector is modified to have reduced CpGs.

In certain embodiments, the AAV vector has a capsid serotype comprising a modified or variant AAV VP1, VP2 and/or VP3 capsid having 90% or more, 95% or more, or 100% sequence identity to AAV1, AAV2, AAV3, AAV4, AAV5, AAV6, AAV7, AAV8, AAV9, AAV10, AAV11, AAV12, Rh10, Rh74 (SEQ ID NO: 35), AAV3B, AAV-2i8, SEQ ID NO: 110, SEQ ID NO: 36, SEQ ID NO: 37, and/or LK03 (SEQ ID NO: 42).

In certain embodiments, the AAV vector comprises one or more ITRs of any of AAV1, AAV2, AAV3, AAV4, AAV5, AAV6, AAV7, AAV8, AAV9, AAV10, AAV11, AAV12, Rh10, Rh74, AAV3B, AAV serotypes, or a combination thereof.

In certain embodiments, the AAV vector comprises the polynucleotide sequence of one of SEQ ID NOs: 21-34, 53-56, and 91-99.

In certain embodiments, the invention relates to a non-viral vector comprising the polynucleotide or expression cassette.

In certain embodiments, the invention relates to a pharmaceutical composition comprising a plurality of the AAV vectors or non-viral vectors in a biologically compatible carrier or excipient. Preferably, the plurality of AAV vectors provide a sufficient amount to achieve a therapeutic effect. However, multiple compositions can be administered to achieve a therapeutic effect.

In certain embodiments, the pharmaceutical composition further comprises empty AAV capsids.

In certain embodiments, the pharmaceutical composition further comprises a surfactant.

In certain embodiments, the invention relates to a method of treating a subject in need of GLA, comprising administering to the subject a therapeutically effective amount of the polynucleotide, the expression cassette, the AAV vector, the non-viral vector, or the pharmaceutical composition, wherein the GLA is expressed in the subject.

In certain embodiments, the subject is human.

In certain embodiments, the subject has Fabry disease.

In certain embodiments, the polynucleotide, expression cassette, AAV vector, non-viral vector, or pharmaceutical composition is administered to the subject intravenously, intra-arterially, intra-cavity, intra-mucosally, or via catheter.

In certain embodiments, the AAV vector is administered to the subject in a range from about 1×10⁸ to about 1×10¹⁴ vector genomes per kilogram (vg/kg) of the weight of the subject.

In certain embodiments, the method reduces, decreases or inhibits one or more symptoms of the need for GLA or of Fabry disease; or prevents or reduces progression or worsening of one or more symptoms of the need for GLA or of Fabry disease; or stabilizes one or more symptoms of the need for GLA or of Fabry disease; or improves one or more symptoms of the need for GLA or of Fabry disease.

In certain embodiments, the invention relates to a cell comprising the polynucleotide or expression cassette.

In certain embodiments, the invention relates to a cell that produces the AAV vector.

In certain embodiments, the invention relates to a method of producing the AAV vector, comprising (a) introducing an AAV vector genome comprising the polynucleotide or expression cassette into a packaging helper cell; and (b) culturing the helper cell under conditions to produce the AAV vector.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings. In the drawings:

FIG. 1 shows a schematic of expression vectors described herein.

FIG. 2A shows bar graphs showing serum GLA enzyme activity in male (left panel) and female (right panel) C57Bl/6 mice that were administered AAV-encapsidated GLA expression cassettes comprising different signal peptides (GLA, SP7, CD300, NOTCH2, ORM1 and TF, indicated along the x-axes), as determined by GLA enzyme activity assay at 4 weeks following AAV transduction.

FIG. 2B is a bar graph showing serum GLA protein levels in female C57Bl/6 mice that were administered AAV-encapsidated GLA expression cassettes having wild-type signal peptide (GLA) or sp7 signal peptide (SP7), measured 6 weeks following AAV transduction; control indicates the level of GLA protein in untreated mice; bar heights indicate the mean of five mice per group; error bars indicate one standard deviation from the mean; t-test was performed to compare serum GLA protein levels in transduced mice versus controls (***p<0.001).

FIG. 3 is a bar graph showing serum GLA enzyme activity in C57Bl/6 and B6; 129-GLA mice that were administered AAV-encapsidated sp7-GLA; bar heights indicate the mean of ten to eleven mice per group; error bars indicate one standard deviation from the mean.

FIG. 4A is a bar graph showing serum GLA enzyme activity as a function of AAV dose escalation in Fabry male mice that were administered AAV-encapsidated sp7-GLA, performed over two separate studies (Study 1 and Study 2), measured 4 weeks post-AAV administration; bar heights represent mean serum GLA activity of five mice per group; error bars indicate one standard deviation from the mean; a horizontal line indicates the basal (non-specific activity) observed in GLA knockout animals as defined as the maximum activity observed across a group of five GLA-/null mice that did not receive AAV-encapsidated sp7-GLA.

FIG. 4B is a graph showing the linearity of dose-response observed for the sp7-GLA AAV vector; data from the GLA-/null male mice treated with AAV-encapsidated sp7-GLA shown in FIG. 4A were replotted as a function of vector genome dosage per mouse; data are fitted with a simple linear regression; the 95% confidence interval is indicated by dotted lines.

FIG. 5A is a bar graph showing GLA activity in the livers of male mice that were administered AAV-encapsidated sp7-GLA; basal levels of activity were determined in samples derived from five GLA-/null untreated mice (controls), and from four age-matched GLA+/null (WT) male mice; bar heights represent mean GLA activity in liver lysates; error bars indicate one standard deviation from the mean.

FIG. 5B is a bar graph showing GLA activity in kidneys of GLA-/null male mice (control), or four age-matched GLA+/null (WT) male mice that were administered AAV-encapsidated sp7-GLA; bar heights represent mean tissue GLA activity; error bars indicate one standard deviation from the mean.

FIG. 6 is a bar graph showing serum GLA activity in C57Bl/6 mice that were administered AAV-encapsidated CpG-free, codon-optimized GLA cassettes described herein; GLA activity in serum was assayed 4 weeks following AAV transduction; bar heights represent mean tissue GLA activity of five mice per group; error bars indicate one standard deviation from the mean.

FIG. 7 is a bar graph showing serum GLA activity in C57Bl/6 mice at 42 days post-AAV administration of codon-optimized GLA variants (sp7-GLA-co4; sp7-GLA-var45), GLA sequence with 7 amino acid substitutions (GLA 7 mut; SPKL0031), or intron-containing variants (intron IgHA; intron VTN1; intron RBP4; intron IgHμ) compared to sp7-GLA (lots A & B).

FIG. 8A is a line graph showing a dose-dependent decrease of serum lyso-GL3 levels in B6; 129-GLA−/− mice administered 4.4E11 vg/kg, 1.4E12 vg/kg, and 4.4E12 vg/kg of AAV-sp7-GLA-co4. Lyso-GL3 levels were analyzed by mass spectrometry over the course of 28 days.

FIG. 8B is a graph showing the linear relationship between α-gal A activity and lyso-GL-3 levels in serum, as measured using an in vitro 4-methylumberlliferyl R-D-galactopyranoside (4-MU-Gal) assay.

FIG. 9A is a bar graph showing the reduction in levels of lyso-GL3 in the sera of B6; 129-GLA−/− mice over the course of one month corresponding to doses of 4.4E11 vg/kg, 1.4E12 vg/kg, and 4.4E12 vg/kg of AAV-sp7-GLA-co4 administered intravenously demonstrating a dose dependent increase in the reduction of lyso-GL3.

FIG. 9B is a bar graph showing the reduction in levels of lyso-GL3 in renal tissue of B6; 129-GLA−/− mice, one month after administration of AAV-sp7-GLA-co4 at doses of 4.4E11 vg/kg, 1.4E12 vg/kg, and 4.4E12 vg/kg, demonstrating a dose dependent reduction of lyso-GL3.

FIG. 9C is a bar graph showing the reduction in levels of lyso-GL3 in cardiac tissue of B6; 129-GLA−/− mice one month after administration of AAV-sp7-GLA-co4 at doses of 4.4E11 vg/kg, 1.4E12 vg/kg, and 4.4E12 vg/kg, demonstrating a dose dependent reduction of lyso-GL3.

FIG. 10A is a graph of serum GLA activity over time post infusion of AAV-sp7-GLA-co4 in non-human primates (cynomolgus macaques).

FIG. 10B is a graph of serum GLA antigen levels over time post infusion of AAV-sp7-GLA-co4 in non-human primates (cynomolgus macaques).

FIG. 11 is a graph of serum GLA antigen levels, measured over a time course of 12 weeks, in GLA knockout (B6; 129-GLA−/−) mice following IV injection with doses of 2E11 vg/kg (up triangle), 4E11 vg/kg (down triangle) or 2E12 vg/kg (diamond) of AAV-sp7-GLA (labeled “AAV-sp7.GLA”). Controls were wildtype mice (circles) or GLA knockout mice (squares) injected with vehicle only (labeled “WT+vehicle” and “GLAko,” respectively). BQL indicates below the quantification limit.

FIG. 12A is a bar graph of GL-3 levels in kidney of GLA knockout (B6; 129-GLA−/−) mice, measured at 1 and 3 months post IV injection of AAV-sp7-GLA (labeled “AAV-sp7.GLA”) at doses of 2E11 vg/kg, 4E11 vg/kg or 2E12 vg/kg. Controls were wildtype mice (labeled “GLA WT”) and GLA knockout mice (labeled “GLA KO”). In each condition, the 3 and 1 month bars are presented from left to right, respectively.

FIG. 12B is a bar graph of lyso-GL-3 levels in the kidney of GLA knockout (B6; 129-GLA−/−) mice, measured at 1 and 3 months post IV injection of AAV-sp7-GLA (labeled “AAV-sp7.GLA”) at doses of 2E11 vg/kg, 4E11 vg/kg or 2E12 vg/kg. Controls were wildtype mice (labeled “GLA WT”) and GLA knockout mice (labeled “GLA KO”). In each condition, the 3 and 1 month bars are presented from left to right, respectively.

FIG. 12C is a bar graph of GL-3 levels in the heart of GLA knockout (B6; 129-GLA−/−) mice, measured at 1 and 3 months post IV injection of AAV-sp7-GLA (labeled “AAV-sp7.GLA”) at doses of 2E11 vg/kg, 4E11 vg/kg or 2E12 vg/kg. Controls were wildtype mice (labeled “GLA WT”) and GLA knockout mice (labeled “GLA KO”). In each condition, the 3 and 1 month bars are presented from left to right, respectively.

FIG. 12D is a bar graph of lyso-GL-3 in the heart of GLA knockout (B6; 129-GLA−/−) mice, measured at 1 and 3 months post IV injection of AAV-sp7-GLA (labeled “AAV-sp7.GLA”) at doses of 2E11 vg/kg, 4E11 vg/kg or 2E12 vg/kg. Controls were wildtype mice (labeled “GLA WT”) and GLA knockout mice (labeled “GLA KO”). In each condition, the 3 and 1 month bars are presented from left to right, respectively.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning commonly understood to one of ordinary skill in the art to which this invention pertains. Otherwise, certain terms cited herein have the meanings as set in the specification.

It must be noted that as used herein and in the appended claims, the singular forms “a,” “an,” and “the” include plural reference unless the context clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element, where such element, step or ingredient is related to the claimed invention. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

All of the features disclosed herein can be combined in any combination. Each feature disclosed in the specification can be replaced by an alternative feature serving a same, equivalent, or similar purpose.

The term “about” as used herein refers to a value within 10% of the underlying parameter (i.e., plus or minus 10%). For example, “about 1:10” means 1.1:10.1 or 0.9:9.9, and about 5 hours means 4.5 hours or 5.5 hours, etc. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “0.01 mg/kg to about 10 mg/kg” body weight of a subject includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a non-viral vector and/or immune cell modulator “two or more” times includes 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc., and so forth.

In an attempt to help the reader of the application, the description has been separated into various paragraphs or sections, or is directed to certain embodiments of the invention. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

The descriptions provided herein include modified nucleic acids encoding GLA, expression cassettes comprising the modified nucleic acids encoding GLA, viral vectors comprising the modified nucleic acids encoding GLA, and non-viral vectors comprising the modified nucleic acids encoding GLA. The invention also includes recombinant AAV particles comprising the modified nucleic acids encoding GLA, non-viral particles comprising the modified nucleic acids encoding GLA, pharmaceutical compositions comprising the modified nucleic acids encoding GLA, methods of treating Fabry disease as well as other lysosomal storage disorders characterized by a GLA deficiency, and the various constructs provided herein for use in treating Fabry disease as well as other lysosomal storage disorders characterized by a GLA deficiency.

### Nucleic Acids

The terms “nucleic acid” and “polynucleotide” are used interchangeably herein to refer to all forms of nucleic acid, oligonucleotides, including deoxyribonucleic acid (DNA) and ribonucleic acid (RNA). In discussing nucleic acids, a sequence or structure of a particular polynucleotide can be described herein according to the convention of providing the sequence in the 5′ to 3′ direction.

In certain embodiments, nucleic acids include genomic DNA, cDNA, antisense DNA/RNA, plasmid DNA, linear DNA, (poly- and oligo-nucleotide), chromosomal DNA, spliced or unspliced mRNA, rRNA, tRNA inhibitory DNA or RNA (RNAi, e.g., small or short hairpin (sh)RNA, microRNA (miRNA), small or short interfering (si)RNA, trans-splicing RNA, or antisense RNA), locked nucleic acid analogue (LNA), oligonucleotide DNA (ODN) single and double stranded, immunostimulating sequence (ISS), riboswitches and ribozymes.

In certain embodiments, nucleic acids include naturally occurring, synthetic, and intentionally modified or altered polynucleotides. Nucleic acids can be single, double, or triplex, linear or circular, and can be of any length.

According to certain embodiments, the polynucleotide is a single-stranded (ssDNA) or a double-stranded DNA (dsDNA) molecule. According to certain embodiments, the polynucleotide is for therapeutic use, e.g., an ssDNA or dsDNA encoding a therapeutic transgene. According to certain embodiments, the dsDNA molecule is a minicircle, a nanoplasmid, open linear duplex DNA or a closed-ended linear duplex DNA (CELiD/ceDNA/doggybone DNA). According to certain embodiments, the ssDNA molecule is a closed circular or an open linear DNA.

A “transgene” is used herein to conveniently refer to a nucleic acid that is intended or has been introduced into a cell or organism. Transgenes include any nucleic acid, such as a heterologous polynucleotide sequence, such as a modified nucleic acid encoding GLA, or a heterologous nucleic acid encoding a protein or peptide or a nucleic acid (e.g., miRNA, etc.).

The term transgene and heterologous nucleic acid/polynucleotide sequences are used interchangeably herein.

As used herein, “α-galactosidase A” or “GLA” or “α-gal A “refers to any nucleic acid or protein of GLA. In certain embodiments, a nucleic acid encoding a GLA encodes a human GLA protein. A full DNA sequence of GLA, including introns and exons, is available in GenBank Accession No. X14448.1. A human GLA enzyme consists of 429 amino acids and is available in GenBank Accession Nos. X14448.1 and U78027. The full-length 429 amino acid human GLA enzyme is a precursor protein that includes a 31-residue signal peptide that is cleaved to result in a mature 398 amino acid subunit containing four N-glycosylation consensus sequences. Unless indicated otherwise by the context employed, reference to GLA include the full-length precursor and mature α-galactosidase A. Examples of GLA include any naturally occurring GLA, mature and variants thereof. An example of a full-length precursor GLA enzyme has the amino acid sequence of SEQ ID NO: 12. An example of a mature GLA enzyme has the amino acid sequence of SEQ ID NO: 100. As used herein, “a nucleic acid encoding a GLA” refers to a recombinant nucleic acid molecule that encodes a protein having at least part of a function or activity of wild type GLA protein. Examples of such nucleic acid include modified nucleic acid sequences encoding GLA.

The term “mutant protein” includes a protein which has a mutation in the gene encoding the protein which results in the inability of the protein to achieve a stable conformation under the conditions normally present in the endoplasmic reticulum (ER). The failure to achieve a stable conformation results in a substantial amount of the enzyme being degraded, rather than being transported to the lysosome. Such a mutation is sometimes called a “conformational mutant.” Such mutations include, but are not limited to, missense mutations, and in-frame small deletions and insertions.

As used herein in certain embodiments, the term “mutant GLA” includes a GLA which has a mutation in a gene encoding GLA which results in the inability of the enzyme to achieve a stable conformation under the conditions normally present in the ER. The failure to achieve a stable conformation results in a substantial amount of the enzyme being degraded, rather than being transported to the lysosome.

As used herein, the terms “modify” and grammatical variations thereof, mean that a nucleic acid or protein deviates from a reference or parental sequence. A modified nucleic acid encoding GLA has been altered compared to reference (e.g., wild-type) or parental nucleic acid. Modified nucleic acids can therefore have substantially the same, greater or less activity or function than a reference or parental nucleic acid, but at least retain partial activity, function and or sequence identity to the reference or parental nucleic acid. The modified nucleic acid can be genetically modified to encode a modified or variant GLA.

A “modified nucleic acid encoding GLA” means that the GLA nucleic acid has alteration compared the parental unmodified nucleic acid encoding GLA. A particular example of a modification is a nucleotide substitution. Nucleotide substitutions can be silent mutations that code for the same amino acid, or missense mutations that code for a different amino acid. Missense mutations can be conservative or non-conservative mutations. Other examples of modifications include, e.g., truncations and insertions. The modified nucleic acid can also include a codon optimized nucleic acid that encodes the same protein as that of the wild-type protein or of the nucleic acid that has not been codon optimized. Codon optimization can be used in a broader sense, e.g., including removing the CpG dinucleotides.

The terms “modification” herein need not appear in each instance of a reference made to a nucleic acid encoding GLA.

In certain embodiments, for a modified nucleic acid encoding GLA, the GLA protein retains at least part of a function or activity of wild type GLA protein. The function or activity of GLA protein includes α-galactosidase A activity, a glycoside hydrolase enzyme that hydrolyses the terminal alpha-galactosyl moieties from glycolipids and glycoproteins. Accordingly, the modified nucleic acids encoding GLA include modified forms so long as the encoded GLA retains some degree or aspect of glycoside hydrolase activity of GLA.

As set forth herein, modified nucleic acids encoding GLA can exhibit different features or characteristics compared to a reference or parental nucleic acid. For example, modified nucleic acids include sequences with 100% identity to a reference nucleic acid encoding GLA as set forth herein, as well as sequences with less than 100% identity to a reference nucleic acid encoding GLA.

The terms “identity,” “homology,” and grammatical variations thereof, mean that two or more referenced entities are the same, when they are “aligned” sequences. Thus, by way of example, when two nucleic acids are identical, they have the same sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In certain embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In further embodiments, the length of the sequence sharing identity is 41 or more contiguous amino acids or nucleic acids, e.g., 42, 43, 44, 45, 45, 47, 48, 49, 50, etc., contiguous amino acids or nucleic acids. In yet further embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

As set forth herein, modified nucleic acids encoding GLA can be distinct from or exhibit 100% identity or less than 100% identity to a reference nucleic acid encoding GLA.

According to certain embodiments, a nucleic acid encoding a GLA is selected from the group consisting of: (1) a polynucleotide having at least 75% sequence identity to the sequence of SEQ ID NO: 15, such as 75% or greater sequence identity, 76% or greater sequence identity, 77% or greater sequence identity, 78% or greater sequence identity, 79% or greater sequence identity, 80% or greater sequence identity, 81% or greater sequence identity, 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 15; (2) a polynucleotide having at least 83% sequence identity to the sequence of SEQ ID NO: 16, such as 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 16; (3) a polynucleotide having at least 85% sequence identity to the sequence of SEQ ID NO: 17, such as 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 17; (4) a polynucleotide having at least 85% sequence identity to the sequence of SEQ ID NO: 18, such as 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 18; and (5) a polynucleotide having at least 82% sequence identity to the sequence of SEQ ID NO: 19, such as 82% or greater sequence identity, 83% or greater sequence identity, 84% or greater sequence identity, 85% or greater sequence identity, 86% or greater sequence identity, 87% or greater sequence identity, 88% or greater sequence identity, 89% or greater sequence identity, 90% or greater sequence identity, 91% or greater sequence identity, 92% or greater sequence identity, 93% or greater sequence identity, 94% or greater sequence identity, 95% or greater sequence identity, 96% or greater sequence identity, 97% or greater sequence identity, 98% or greater sequence identity, 99% or greater sequence identity, 99.5% or greater sequence identity to the sequence of SEQ ID NO: 19. According to certain embodiments, a nucleic acid encoding a GLA has 100% sequence identity to any one of the sequences of SEQ ID NOs: 15-19.

In certain embodiments, the nucleic acid encoding GLA further comprises one or more introns positioned anywhere within the nucleic acid encoding the GLA. In certain embodiments, an intron is positioned between nucleotides 78 and 79 of the nucleic acid encoding the GLA, wherein the nucleotide positions are given in reference to the coding sequence of GLA having a sequence of SEQ ID NO: 14.

In certain embodiments, the intron is selected from the group consisting of introns from vitronectin1 (VTN1) gene, retinol binding protein 4 (RBP4) gene, mouse IgG heavy chain A (IgHA) gene, and mouse IgG heavy chain μ (IgHμ) gene. In certain embodiments, the one or more introns are selected from the sequences of SEQ ID NOs: 49-52.

In certain embodiments, the nucleic acid encoding GLA has a sequence of any one of SEQ ID NOs: 43-46.

According to certain embodiments, a nucleic acid of the invention encodes a GLA having the amino acid sequence of SEQ ID NO: 100.

In certain embodiments, the GLA comprises the amino acid sequence of SEQ ID NO: 100 with one amino acid substitution selected from the group consisting of Gln57Lys, Gln111Glu, Lys213Glu, Lys237Gln, Phe248Thr, Gly334Glu, and Gly346Asn. In certain embodiments, the GLA comprises the amino acid sequence of SEQ ID NO: 100 with any two amino acid substitutions selected from the group consisting of Gln57Lys, Gln111Glu, Lys213Glu, Lys237Gln, Phe248Thr, Gly334Glu, and Gly346Asn. In certain embodiments, the GLA comprises the amino acid sequence of SEQ ID NO: 100 with any three amino acid substitutions selected from the group consisting of Gln57Lys, Gln111Glu, Lys213Glu, Lys237Gln, Phe248Thr, Gly334Glu, and Gly346Asn. In certain embodiments, the GLA comprises the amino acid sequence of SEQ ID NO: 100 with any four amino acid substitutions selected from the group consisting of Gln57Lys, Gln111Glu, Lys213Glu, Lys237Gln, Phe248Thr, Gly334Glu, and Gly346Asn. In certain embodiments, the GLA comprises the amino acid sequence of SEQ ID NO: 100 with any five amino acid substitutions selected from the group consisting of Gln57Lys, Gln111Glu, Lys213Glu, Lys237Gln, Phe248Thr, Gly334Glu, and Gly346Asn. In certain embodiments, the GLA comprises the amino acid sequence of SEQ ID NO: 100 with any six amino acid substitutions selected from the group consisting of Gln57Lys, Gln111Glu, Lys213Glu, Lys237Gln, Phe248Thr, Gly334Glu, and Gly346Asn. In certain embodiments, the GLA comprises the amino acid sequence of SEQ ID NO: 100 with the seven amino acid substitutions of Gln57Lys, Gln111Glu, Lys213Glu, Lys237Gln, Phe248Thr, Gly334Glu, and Gly346Asn. In certain embodiments, the GLA comprises the amino acid sequence of SEQ ID NO: 48.

In certain embodiments, the nucleic acid encoding GLA comprises the coding sequence of SEQ ID NO: 47.

Modified nucleic acids encoding GLA that exhibit different features or characteristics compared to a reference or parental nucleic acid include substitutions of nucleotides. For example, modified nucleic acids encoding GLA include nucleic acids with a reduced number of CpG dinucleotides compared to a reference nucleic acid encoding GLA, referred to as CpG-reduced nucleic acids.

As used herein, the phrase “CpG-reduced” or “CpG-depleted” refers to a nucleic acid sequence which is generated, either synthetically or by mutation of a nucleic acid sequence, such that one or more of the CpG dinucleotides (or motifs) are removed from the nucleic acid sequence. In certain embodiments, all CpG motifs are removed to provide what is termed herein as a modified CpG-free sequence. The CpG motifs are suitably reduced or eliminated not just in a coding sequence (e.g., a transgene), but also in the non-coding sequences, including, e.g., 5′ and 3′ untranslated regions (UTRs), promoter, enhancer, signal peptides, polyA, ITRs, introns, and any other sequences present in the polynucleotide molecule.

According to certain embodiments, a nucleic acid encoding a GLA contains less than 14 CpG dinucleotides, such as 13, 12, 11, 10, 9, 8, 7, 6, 5, 4, 3, 2, 1, or 0 CpG dinucleotides.

The phrase “consisting essentially of” when referring to a particular nucleotide sequence or amino acid sequence means a sequence having the properties of the sequence of a given SEQ ID NO. For example, when used in reference to an amino acid sequence, the phrase includes the sequence per se and molecular modifications that would not affect the basic and novel characteristics of the sequence.

Nucleic acids, expression vectors, AAV vector genomes, non-viral vectors, plasmids, including modified nucleic acids encoding GLA of the invention can be prepared by using recombinant DNA technology methods. The availability of nucleotide sequence information enables preparation of isolated nucleic acid molecules of the invention by a variety of means. Nucleic acids encoding GLA can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids can be maintained as DNA in any convenient cloning vector. In certain embodiments, clones are maintained in a plasmid cloning/expression vector, such as pBluescript (Stratagene, La Jolla, CA), which is propagated in a suitable E. coli host cell. Alternatively, nucleic acids can be maintained in vector suitable for expression in mammalian cells, for example, an AAV vector. In cases where post-translational modification affects protein function, nucleic acid molecule can be expressed in mammalian cells.

### Expression Cassettes

The invention also provides expression cassettes comprising the polynucleotides comprising the nucleic acids encoding GLA as described herein, operably linked to an expression control element. In certain embodiments, the expression cassette comprises a nucleic acid encoding a GLA, wherein the nucleic acid is selected from the group consisting of: (1) a polynucleotide having at least 75% sequence identity (e.g., 75%-100% identity) to the sequence of SEQ ID NO: 15, (2) a polynucleotide having at least 84% sequence identity (e.g., 84%-100% identity) to the sequence of SEQ ID NO: 16, (3) a polynucleotide having at least 86% sequence identity (e.g., 86%-100% identity) to the sequence of SEQ ID NO: 17, (4) a polynucleotide having at least 86% sequence identity (e.g., 86%-100% identity) to the sequence of SEQ ID NO: 18, and (5) a polynucleotide having at least 83% sequence identity (e.g., 83%-100% identity) to the sequence of SEQ ID NO: 19.

In certain embodiments, the GLA comprises the amino acid sequence of SEQ ID NO: 100.

In certain embodiments, the expression cassette comprises an appropriate secretory signal sequence or signal peptide that will allow the secretion of the polypeptide encoded by the polynucleotide molecule of the instant invention. As used herein, the term “secretory signal sequence” or “signal peptide” or variations thereof are intended to refer to amino acid sequences that function to enhance secretion of an operably linked polypeptide from the cell as compared with the level of secretion seen with the native polypeptide. Signal peptides are short amino acid sequences, typically less than 20 amino acids in length, that direct proteins to or through the endoplasmic reticulum secretory pathway. By “enhanced” secretion, it is meant that the relative proportion of the polypeptide synthesized by the cell that is secreted from the cell is increased; it is not necessary that the absolute amount of secreted protein is also increased. In certain embodiments, essentially all (i.e., at least 95%, 97%, 98%, 99% or more) of the polypeptide is secreted. It is not necessary, however, that essentially all or even most of the polypeptide is secreted, as long as the level of secretion is enhanced as compared with the native polypeptide. Generally, secretory signal sequences are cleaved within the endoplasmic reticulum and, in certain embodiments, the secretory signal sequence is cleaved prior to secretion. It is not necessary, however, that the secretory signal sequence is cleaved as long as secretion of the polypeptide from the cell is enhanced and the polypeptide is functional. Thus, in certain embodiments, the secretory signal sequence is partially or entirely retained. The secretory signal sequence can be derived in whole or in part from the secretory signal of a secreted polypeptide (i.e., from the precursor) and/or can be in whole or in part synthetic. The length of the secretory signal sequence is not critical; generally, known secretory signal sequences are from about 10-15 to 50-60 amino acids in length. Further, known secretory signals from secreted polypeptides can be altered or modified (e.g., by substitution, deletion, truncation or insertion of amino acids) as long as the resulting secretory signal sequence functions to enhance secretion of an operably linked polypeptide. The secretory signal sequences of the instant invention can comprise, consist essentially of or consist of a naturally occurring secretory signal sequence or a modification thereof. Numerous secreted proteins and sequences that direct secretion from the cell are known in the art. The secretory signal sequence of the instant invention can further be in whole or in part synthetic or artificial. Synthetic or artificial secretory signal peptides are known in the art, see, e.g., Barash et al., Biochem. Biophys. Res. Comm. 294:835-42 (2002).

Any suitable signal peptide known to those skilled in the art in view of the present disclosure can be used in the invention. Examples of signal peptides include, but are not limited to, those found from the Signal Peptide Database (website: www.signalpeptide.de/). Examples of signal peptides suitable for the present invention include, but are not limited to, wild-type GLA signal peptide, a human chymotrypsinogen B2 signal peptide (“sp7”; 18 amino acid signal peptide of NCBI reference sequence NP_001020371), alpha 2-HS-glycoprotein (AHSG) signal peptide, CD300 signal peptide, lysosome-associated membrane glycoprotein 1 (LAMP1) signal peptide, Notch 2 signal peptide, orosomucoid 1 (ORM1) signal peptide, transferrin (TF) signal peptide, secrecon (artificial signal sequence described in Barash et al., Biochem Biophys Res Commun. 2002; 294: 835-842), mouse IgKVIII, human IgKVIII, CD33, tPA, a-1 antitrypsin signal peptide, and native secreted alkaline phosphatase (SEAP). Any conventional signal sequence that directs proteins through the endoplasmic reticulum secretory pathway, including variants of the above mentioned signal peptides, can be used in the present invention.

In certain embodiments, the signal peptide is an endogenous or native GLA signal peptide or a variant thereof.

In certain embodiments, the signal peptide is a heterologous signal peptide or a variant thereof.

In certain embodiments, the signal peptide has a coding sequence of any one of SEQ ID NOs: 1-11 and 13.

In certain embodiments, the signal peptide has an amino acid sequence of any one of SEQ ID NOs: 41 and 57-63.

In certain embodiments, the expression cassette comprises a nucleic acid encoding a signal peptide sequence positioned at the 5′ end of the nucleic acid encoding the GLA. In certain embodiments, the signal peptide is a human chymotrypsinogen B2 signal peptide. In certain embodiments, the signal peptide is a human chymotrypsinogen B2 signal peptide having an amino acid sequence of SEQ ID NO: 41. In certain embodiments, the signal peptide is a human chymotrypsinogen B2 signal peptide having a coding sequence of any one of SEQ ID NOs: 1-5.

In certain embodiments, the polynucleotide encodes a GLA having a sequence of any one of SEQ ID NOs: 101-109.

In certain embodiments, the polynucleotide comprises a sequence of any one of SEQ ID NOs: 64-81.

In certain embodiments, an expression control element is positioned 5′ of a nucleic acid encoding a GLA.

The term “expression cassette”, as used herein, refers to a nucleic acid construct comprising nucleic acid elements sufficient for the expression of the polynucleotide molecule of the instant invention. Typically, an expression cassette comprises the polynucleotide molecule of the instant invention operably linked to a promoter sequence.

An “expression control element” refers to nucleic acid sequence(s) that influence expression of an operably linked nucleic acid. Expression control elements as set forth herein include promoters and enhancers. Vector sequences including AAV vectors and non-viral vectors can include one or more “expression control elements.” Typically, such elements are included to facilitate proper heterologous polynucleotide transcription and as appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but can also act in trans.

Expression control can be affected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100-500, or more nucleotides from the polynucleotide), even at considerable distances. Nevertheless, owing to the length limitations of AAV vectors, expression control elements in AAV vectors will typically be within 1 to 1000 nucleotides from the transcription start site of the heterologous nucleic acid.

Functionally, expression of an operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

The term “operably linked” means that the regulatory sequences necessary for expression of a nucleic acid sequence are placed in the appropriate positions relative to the sequence so as to mediate expression of the nucleic acid sequence. This same definition is sometimes applied to the arrangement of nucleic acid sequences and transcription control elements (e.g., promoters, enhancers, and termination elements) in an expression vector, e.g., rAAV vector or non-viral vector. Encoding sequences can be operably linked to regulatory sequences in sense or antisense orientation. In certain embodiments, the promoter is a heterologous promoter.

The term “heterologous promoter”, as used herein, refers to a promoter that is not found to be operably linked to a given encoding sequence in nature. In certain embodiments, an expression cassette can comprise additional elements, for example, an intron, an enhancer, a polyadenylation site, a woodchuck response element (WRE), and/or other elements known to affect expression levels of the encoding sequence.

As used herein, the term “promoter” refers to a nucleotide sequence capable of controlling the expression of a coding sequence or functional RNA. In general, nucleic acid molecules of the instant invention are located 3′ of a promoter sequence. In certain embodiments, a promoter sequence consists of proximal and more distal upstream elements and can comprise an enhancer element.

An “enhancer” as used herein can refer to a sequence that is located adjacent to the heterologous nucleic acid. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence. Enhancer elements typically increase expression of an operably linked nucleic acid afforded by a promoter element.

An expression construct can comprise regulatory elements which serve to drive expression in a particular cell or tissue type. Expression control elements (e.g., promoters) include those active in a particular tissue or cell type, referred to herein as a “tissue-specific expression control element/promoter.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Green, M. and Sambrook, J. (2012) Molecular Cloning: A Laboratory Manual. 4th Edition, Vol II, Cold Spring Harbor Laboratory Press, New York; and Ausubel et al. (2010) Current protocols in molecular biology, John Wiley & Sons, New York).

The incorporation of tissue specific regulatory elements in the expression constructs provides for at least partial tissue tropism for the expression of a heterologous nucleic acid encoding a protein or inhibitory RNA. Examples of promoters that are active in liver are the transthyretin (TTR) gene promoter; human alpha 1-antitrypsin (hAAT) promoter; the apolipoprotein A-I promoter; albumin, Miyatake, et al., J. Virol., 71:5124-32 (1997); hepatitis B virus core promoter, Sandig, et al., Gene Ther. 3: 1002-9 (1996); alpha-fetoprotein (AFP), Arbuthnot, et al., Hum. Gene. Ther., 7:1503-14 (1996); human Factor IX promoter; thyroxin binding globulin (TBG) promoter; TTR minimal enhancer/promoter; alpha-antitrypsin promoter; LSP (845 nt) (requires intronless scAAV); and LSP1 promoter, among others. An example of an enhancer active in liver is apolipoprotein E (apoE) HCR-1 and HCR-2 (Allan et al., J Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al., Cell, 41:521-530 (1985)), the SV40 promoter, the dihydrofolate reductase promoter, the cytoplasmic b-actin promoter and the phosphoglycerate kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268: 1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J Clin. Invest. 100:2865-2872 (1997); Rivera, et al., Nat. Medicine. 2:1028-1032 (1996)). Other regulatable control elements which can be useful in this context are those which are regulated by a specific physiological state, e.g., temperature, acute phase, development.

Other examples of promoters include, but are not limited to, the phosphoglycerate kinase (PKG) promoter, CAG (composite of the CMV enhancer, the chicken beta actin promoter (CBA) and the rabbit beta globin intron) and other constitutive promoters, NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, SFFV promoter, rous sarcoma virus (RSV) promoter, rat insulin promoter, TBG promoter and other liver-specific promoters, the desmin promoter and similar muscle-specific promoters, the EF1-alpha promoter, synthetic promoters, hybrid promoters, promoters with multi-tissue specificity, and the like, all of which are promoters well known and readily available to those of skill in the art. Other promoters can be of human origin or from other species, including from mice.

Expression control elements also include the native elements(s) for the heterologous polynucleotide. A native control element (e.g., promoter) can be used when it is desired that expression of the heterologous polynucleotide should mimic the native expression. The native element can be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences can also be used.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (poly A) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In certain embodiments, a filler/stuffer nucleic acid sequence is an untranslated (non-protein encoding) segment of nucleic acid. For a nucleic acid sequence less than 4.7 kb, the filler or stuffer polynucleotide sequence has a length that when combined (e.g., inserted into a vector) with the sequence has a total length between about 3.0-5.5 kb, or between about 4.0-5.0 kb, or between about 4.3-4.8 kb.

In certain embodiments, the expression control element comprises an ApoE/hAAT enhancer/promoter sequence positioned 5′ of the nucleic acid encoding GLA. In certain embodiments, the ApoE/hAAT enhancer/promoter sequence is CpG-reduced compared to wild-type ApoE/hAAT enhancer/promoter sequence. In certain embodiments, the ApoE/hAAT enhancer/promoter sequence has a sequence of SEQ ID NO: 38.

In certain embodiments, the expression cassette includes a poly-adenylation (polyA) sequence positioned 3′ of the nucleic acid encoding a GLA. In certain embodiments, the polyA sequence comprises a bovine growth hormone (bGH) polyadenylation sequence. In certain embodiments, the bGH polyadenylation sequence is CpG-reduced compared to wild-type bGH polyadenylation sequence. In certain embodiments, the bGH polyadenylation sequence has a sequence of SEQ ID NO: 20.

In certain embodiments, the expression cassette further comprises an intron positioned between the 3′ end of the expression control element and the 5′ end of the nucleic acid encoding a GLA. In certain embodiments, the intron is an hBB2m1 intron. In certain embodiments, the hBB2m1 intron sequence is CpG-reduced compared to wild-type hBB2m1 intron sequence. In certain embodiments, the hBB2m1 intron sequence has a sequence of SEQ ID NO: 39.

In certain embodiments, the expression cassette further comprises one or more introns positioned anywhere within the nucleic acid encoding a GLA. In certain embodiments, an intron is positioned at a site within the nucleic acid encoding the GLA that matches the consensus nucleotide sequence of MAG/G, where M is Adenine or Cytosine, and the “/” denotes the site of the intron insertion. In certain embodiments, an intron is positioned between nucleotides 78 and 79 of the nucleic acid encoding the GLA, wherein the nucleotide positions are given in reference to the coding sequence of GLA having a sequence of SEQ ID NO: 14. Any suitable intron known to those skilled in the art in view of the present disclosure can be used in the invention. Examples of suitable introns include, but are not limited to, introns from vitronectin1 (VTN1) gene, retinol binding protein 4 (RBP4) gene, mouse IgG heavy chain A (IgHA) gene, and mouse IgG heavy chain μ (IgHμ) gene. In certain embodiments, the one or more introns are selected from the sequences of any of SEQ ID NOs: 49-52.

In certain embodiments, the expression cassette has a sequence of any one of SEQ ID NOs: 21-34, 53-56, and 91-99. In certain embodiments, the expression cassette has a sequence of at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, at least 99.5% sequence identity, or 100% sequence identity to the sequence of any one of SEQ ID NOs: 21-34, 53-56 and 91-99.

### Gene Transfer Systems

### Viral Vectors

The invention further provides viral vectors such as adeno-associated virus (AAV) vectors comprising polynucleotides comprising the nucleic acids encoding GLA as set forth herein.

The term “vector” or “gene transfer vector” as used herein, refers to a nucleic acid molecule comprising a gene of interest. Examples of vectors include, but are not limited to, viral vectors delivered by viral particles or virus-like particles (VLPs) that resemble viral particles but are non-infectious, such as retroviral, adenoviral, adeno-associated viral, and lentiviral particles or VLPs; and non-viral vectors delivered by non-viral gene transfer systems, such as microinjection, electroporation, liposomes, large natural polymers, large synthetic polymers, and polymers comprised of both natural and synthetic components.

A vector nucleic acid sequence generally contains at least an origin of replication for propagation in a cell and optionally additional elements, such as a heterologous polynucleotide sequence, expression control element (e.g., a promoter, enhancer), intron, an inverted terminal repeat (ITR), selectable marker (e.g., antibiotic resistance), polyadenylation signal.

As used herein, the term “gene transfer system” refers to any means of delivering a composition comprising a nucleic acid sequence to a cell or tissue. For example, a gene transfer system can be a viral gene transfer system, e.g., intact viruses, modified viruses and VLPs to facilitate delivery of a viral vector to a desired cell or tissue. A gene transfer system can also be a non-viral delivery system that does not comprise viral coat protein or form a viral particle or VLP, e.g., liposome-based systems, polymer-based systems, protein-based systems, metallic particle-based systems, peptide cage systems, etc.

A viral vector is derived from or based upon one or more nucleic acid elements that comprise a viral genome. Particular viral vectors include lentiviral and adeno-associated virus (AAV) vectors.

The term “recombinant,” as a modifier of vector, such as recombinant AAV (rAAV) vector, as well as a modifier of sequences such as recombinant polynucleotides and polypeptides, means that the compositions have been manipulated (i.e., engineered) in a fashion that generally does not occur in nature. Although the term “recombinant” is not always used herein in reference to AAV vectors, as well as sequences such as polynucleotides, recombinant forms including polynucleotides, are expressly included in spite of any such omission.

A “recombinant AAV vector” or “rAAV” is derived from the wild type genome of AAV by using molecular methods to remove the wild type genome from the AAV genome, and replacing with a non-native nucleic acid sequence, referred to as a heterologous nucleic acid. Typically, for AAV one or both inverted terminal repeat (ITR) sequences of AAV genome are retained in the AAV vector. rAAV is distinguished from an AAV genome, since all or a part of the AAV genome has been replaced with a non-native sequence with respect to the AAV genomic nucleic acid. Incorporation of a non-native sequence therefore defines the AAV vector as a “recombinant” vector, which can be referred to as a “rAAV vector.”

An rAAV sequence can be packaged, referred to herein as a “particle,” for subsequent infection (transduction) of a cell, ex vivo, in vitro or in vivo. Where a recombinant AAV vector sequence is encapsidated or packaged into an AAV particle, the particle can also be referred to as an “rAAV vector” or “rAAV particle.” Such rAAV particles include proteins that encapsidate or package the vector genome and in the case of AAV, they are referred to as capsid proteins.

A vector “genome” refers to the portion of the recombinant plasmid sequence that is ultimately packaged or encapsidated to form a viral (e.g., rAAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid can be referred to as the “plasmid backbone,” which is important for cloning and amplification of the plasmid, a process that is needed for propagation and recombinant virus production. Except for possible 3′ ITR and/or 5′ ITR cloning remnants the plasmid backbone is not itself packaged or encapsidated into virus (e.g., AAV) particles. Thus, a vector “genome” refers to the polynucleotide that is packaged or encapsidated by virus (e.g., AAV).

Host cells for producing recombinant AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Other host cell lines appropriate for recombinant AAV production are described in International Application PCT/2017/024951, the disclosure of which is herein incorporated in its entirety.

In certain embodiments, AAV helper functions are introduced into the host cell by transfecting the host cell with an AAV helper construct either prior to, or concurrently with, the transfection of an AAV expression vector. A host cell having AAV helper functions can be referred to as a “helper cell” or “packaging helper cell.” AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

Methods of generating recombinant AAV particles capable of transducing mammalian cells are known in the art. For example, recombinant AAV particles can be produced as described in U.S. Pat. No. 9,408,904; and International Applications PCT/US2017/025396 and PCT/US2016/064414, the disclosures of which are herein incorporated in their entirety.

The invention provides cells comprising nucleic acids encoding GLA, cells comprising expression cassettes comprising the polynucleotides comprising the nucleic acids encoding GLA, cells comprising viral vectors such as AAV vectors comprising nucleic acids encoding GLA, and cells comprising non-viral vectors comprising polynucleotides comprising the nucleic acids encoding GLA. In certain embodiments, the cell produces a viral vector. In certain embodiments, the cell produces an AAV vector as set forth herein.

Also provided are methods of producing viral vectors such as AAV vectors as set forth herein. In certain embodiments, a method of producing AAV vectors includes: introducing an AAV vector genome comprising a nucleic acid encoding GLA or expression cassette comprising a nucleic acid encoding GLA as set forth herein into a packaging helper cell; and culturing the helper cell under conditions to produce the AAV vectors. In certain embodiments, a method of producing AAV vectors includes: introducing a nucleic acid encoding GLA or expression cassette comprising a nucleic acid encoding GLA as set forth herein into a packaging helper cell; and culturing the helper cells under conditions to produce the AAV vector.

In certain embodiments, the cells are mammalian cells.

In certain embodiments, cells for vector production provide helper functions, such as AAV helper functions, that package the vector into a viral particle. In a particular aspect, the helper functions are Rep and/or Cap proteins for AAV vector packaging. In certain embodiments, cells for vector production can be stably or transiently transfected with polynucleotide(s) encoding Rep and/or Cap protein sequence(s). In certain embodiments, cells for vector production provide Rep78 and/or Rep68 proteins. In such cells, the cells can be stably or transiently transfected with Rep78 and/or Rep68 proteins polynucleotide encoding sequence(s).

In certain embodiments, cells for vector production are human embryonic kidney cells. In a particular aspect, cells for vector production are HEK-293 cells.

The term “transduce” and grammatical variations thereof refer to introduction of a molecule such as an rAAV vector into a cell or host organism. The heterologous nucleic acid/transgene may or may not be integrated into genomic nucleic acid of the recipient cell. The introduced heterologous nucleic acid can also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation, for example, of a nucleic acid (e.g., a transgene) into the cell. Thus, a “transduced” cell is a cell into which, or a progeny thereof in which an exogenous nucleic acid has been introduced. The cell(s) can be propagated and the introduced protein expressed. For gene therapy uses and methods, a transduced cell can be in a subject.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, or cell membrane.

The term “isolated” does not exclude combinations produced by the hand of man, for example, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g., chromatographic methods, agarose or polyacrylamide gel electrophoresis, HPLC analysis, and the like).

Recombinant AAV vector, as well as methods and uses thereof, include any viral strain or serotype. As a non-limiting example, a recombinant AAV vector can be based upon any AAV genome, such as LK03, AAV1, AAV2, AAV3, AAV4, AAV5, AAV6, AAV7, AAV8, AAV9, AAV10, AAV11, AAV 12, Rh10, Rh74, AAV3B or AAV-2i8, for example. Such vectors can be based on the same strain or serotype (or subgroup or variant), or be different from each other. As a non-limiting example, a recombinant AAV vector based upon a particular serotype genome can be identical to the serotype of the capsid proteins that package the vector. In addition, a recombinant AAV vector genome can be based upon an AAV serotype genome distinct from the serotype of the AAV capsid proteins that package the vector. For example, the AAV vector genome can be based upon AAV2, whereas at least one of the three capsid proteins could be an LK03, AAV1, AAV3, AAV4, AAV5, AAV6, AAV7, AAV8, AAV9, AAV10, AAV11, AAV12, Rh10, Rh74, AAV3B or AAV-2i8, or variant thereof.

In certain embodiments, adeno-associated virus (AAV) vectors include LK03, AAV1, AAV2, AAV3, AAV4, AAV5, AAV6, AAV7, AAV8, AAV9, AAV10, AAV11, AAV12, Rh10, Rh74, AAV3B and AAV-2i8, as well as variants (e.g., capsid variants, such as amino acid insertions, additions, substitutions and deletions) thereof, for example, as set forth in WO 2013/158879 (International Application PCT/US2013/037170), WO 2015/013313 (International Application PCT/US2014/047670; disclosing RHM4-1, RHM15-1, RHM15-2, RHM15-3/RHM15-5, RHM15-4 and RHM15-6), US 2013/0059732 (U.S. Pat. No. 9,169,299, discloses LK01, LK02, LK03, etc.), and WO 2016/210170, the disclosures of which are herein incorporated in their entirety.

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants might not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that can be within a subgroup or a variant of a given serotype.

As set forth herein, AAV capsid proteins can exhibit less than 100% sequence identity to a reference or parental AAV serotype such as AAV1, AAV2, AAV3, AAV4, AAV5, AAV6, AAV7, AAV8, AAV9, AAV10, AAV11, AAV12, Rh10, Rh74 (SEQ ID NO: 35), AAV3B, LK03 (SEQ ID NO: 42), AAV-2i8, the sequence of SEQ ID NO: 110, the sequence of SEQ ID NO: 36, and/or the sequence of SEQ ID NO: 37, but are distinct from and not identical to known AAV genes or proteins, such as AAV1, AAV2, AAV3, AAV4, AAV5, AAV6, AAV7, AAV8, AAV9, AAV10, AAV11, AAV12, Rh10, Rh74 (SEQ ID NO: 35), AAV3B, LK03 (SEQ ID NO: 42) or AAV-2i8. In certain embodiments, a modified/variant AAV capsid protein includes or consists of a sequence at least 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 99.9% identical to a reference or parental AAV capsid protein, such as AAV1, AAV2, AAV3, AAV4, AAV5, AAV6, AAV7, AAV8, AAV9, AAV10, AAV11, AAV12, Rh10, Rh74 (SEQ ID NO: 35), AAV3B, LK03 (SEQ ID NO: 42), AAV-2i8, the sequence of SEQ ID NO: 110, the sequence of SEQ ID NO: 36, the sequence of SEQ ID NO: 37, and/or the sequence of SEQ ID NO: 42.

In certain embodiments, a viral vector such as an adeno-associated virus (AAV) vector comprises any of the polynucleotides comprising the nucleic acids encoding GLA as set forth herein operably linked to an expression control element.

In certain embodiments, a viral vector such as an adeno-associated virus (AAV) vector comprises any of the expression cassettes comprising the polynucleotides comprising the nucleic acids encoding GLA as set forth herein.

In certain embodiments, an AAV vector comprises: one or more of an AAV capsid; and one or more AAV inverted terminal repeats (ITRs), wherein the AAV ITR(s) flanks the 5′ or 3′ terminus of the polynucleotide or the expression cassette.

In certain embodiments, an AAV vector further comprises an intron positioned 5′ or 3′ of one or more ITRs.

In certain embodiments, an AAV vector comprising at least one or more ITRs or an intron has the one or more ITRs or intron modified to have reduced CpGs.

In certain embodiments, an AAV vector of the invention is delivered via a non-viral delivery system, including for example, encapsulated in a lipid nanoparticle (LNP).

### Non-Viral Methods

In certain embodiments, the polynucleotides and expression cassettes of the invention are delivered or administered with a non-viral delivery system. Non-viral delivery systems include for example, chemical methods, such as non-viral vectors, or extracellular vesicles and physical methods, such as gene gun, electroporation, particle bombardment, ultrasound utilization and magnetofection.

Recombinant cells capable of expressing the GLA sequences of the invention can be used for delivery or administration.

Naked DNA such as minicircles and transposons can be used for administration or delivery or lentiviral vectors. Additionally, gene editing technologies such as zinc finger nucleases, meganucleases, TALENs, and CRISPR can also be used to deliver the coding sequence of the invention.

In certain embodiments, the polynucleotides and expression cassettes of the invention are delivered as naked DNA, minicircles, transposons, of closed-ended linear duplex DNA.

In certain embodiments, the polynucleotides and expression cassettes of the invention are delivered or administered in AAV vector particles, or other viral particles, that are further encapsulated or complexed with liposomes, nanoparticles, lipid nanoparticles, polymers, microparticles, microcapsules, micelles, or extracellular vesicles.

In certain embodiments, the polynucleotides and expression cassettes of the invention are delivered or administered with non-viral vectors.

As used herein, a “non-viral vector” refers to a vector that is not delivered by viral particles or by viral-like particles (VLPs). According to certain embodiments, a non-viral vector is a vector that is not delivered by a capsid. The vector can be encapsulated, admixed, or otherwise associated with the non-viral delivery nanoparticle.

Any suitable non-viral delivery system known to those skilled in the art in view of the present disclosure can be used in the invention. The non-viral delivery nanoparticle can be, for example, a lipid-based nanoparticle, a polymer-based nanoparticle, a protein-based nanoparticle, a microparticle, a microcapsule, a metallic particle-based nanoparticle, a peptide cage nanoparticle, etc.

A non-viral delivery nanoparticle of the instant invention can be constructed by any method known in the art, and a non-viral vector of the instant invention comprising a nucleic acid molecule comprising a therapeutic transgene can be constructed by any method known in the art.

### Lipid-Based Delivery Systems

Lipid-based delivery systems are well known in the art, and any suitable lipid-based delivery system known to those skilled in the art in view of the present disclosure can be used in the invention. Examples of lipid-based delivery systems include, e.g., liposomes, lipid nanoparticles, micelles, or extracellular vesicles.

A “lipid nanoparticle” or “LNP” refers to a lipid-based vesicle useful for delivery of AAV and non-viral vectors having dimensions on the nanoscale, i.e., from about 10 nm to about 1000 nm, or from about 50 to about 500 nm, or from about 75 to about 127 nm. Without being bound by theory, an LNP is believed to provide a polynucleotide, expression cassette, AAV vector, or non-viral vector with partial or complete shielding from the immune system. Shielding allows delivery of the polynucleotide, expression cassette, AAV vector, or non-viral vector to a tissue or cell while avoiding inducing a substantial immune response against the polynucleotide, expression cassette, AAV vector, or non-viral vector in vivo. Shielding can also allow repeated administration without inducing a substantial immune response against the polynucleotide, expression vector, AAV vector, or non-viral vector in vivo (e.g., in a subject such as a human). Shielding can also improve or increase polynucleotide, expression cassette, AAV vector, or non-viral vector delivery efficiency in vivo.

The pI (isoelectric point) of AAV is in a pH range from about 6 to about 6.5. Thus, the AAV surface carries a slight negative charge. As such it can be beneficial for an LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, the disclosures of which are herein incorporated in their entirety.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids can also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

Cationic lipids can include, without limitation, l,2-dilinoleyloxy-N,N-dimethylaminopropane (DLinDMA), 1,2-dilinolenyloxy-N,N-dimethylaminopropane (DLenDMA), 1,2-di-y-linolenyloxy-N,N-dimethylami nopropane (g-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include, but are not limited to 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, y-DLen-C2K-DMA, and (DLin-MP-DMA) (also known as 1-B11).

Still further cationic lipids can include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.C1), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-l-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-l-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECT AMINE® (comprising DOSPA and DOPE, available from GIBCO/BRL).

In certain embodiments, cationic lipid can be present in an amount from about 10% by weight of the LNP to about 85% by weight of the lipid nanoparticle, or from about 50% by weight of the LNP to about 75% by weight of the LNP.

Sterols can confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, most commonly cholesterol. Phytosterols can include campesterol, sitosterol, and stigmasterol. Sterols also include sterol-modified lipids, such as those described in U.S. Patent Application Publication 2011/0177156, the disclosure of which is herein incorporated in its entirety. In certain embodiments, a sterol can be present in an amount from about 5% by weight of the LNP to about 50% by weight of the lipid nanoparticle or from about 10% by weight of the LNP to about 25% by weight of the LNP.

LNP can comprise a neutral lipid. Neutral lipids can comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized by well-known techniques. In certain embodiments, lipids containing saturated fatty acids with carbon chain lengths in the range of C14 to C22 can be used. In another group of embodiments, lipids with mono or diunsaturated fatty acids with carbon chain lengths in the range of C14 to C22 are used. Additionally, lipids having mixtures of saturated and unsaturated fatty acid chains can be used. Exemplary neutral lipids include, without limitation, l,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids can also be composed of sphingomyelin, dihydrosphingomyelin, or phospholipids with other head groups, such as serine and inositol.

In certain embodiments, the neutral lipid can be present in an amount from about 0.1% by weight of the lipid nanoparticle to about 75% by weight of the LNP, or from about 5% by weight of the LNP to about 15% by weight of the LNP.

LNP encapsulated nucleic acids, expression cassettes, AAV vectors, and non-viral vectors can be incorporated into pharmaceutical compositions, e.g., a pharmaceutically acceptable carrier or excipient. Such pharmaceutical compositions are useful for, among other things, administration and delivery of LNP encapsulated nucleic acids, expression cassettes, AAV vectors, and non-viral vectors to a subject in vivo or ex vivo.

Preparations of LNP can be combined with additional components. Non-limiting examples include polyethylene glycol (PEG) and sterols.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments, PEG can be a polyethylene glycol with an average molecular weight of about 550 to about 10,000 daltons and is optionally substituted by alkyl, alkoxy, acyl or aryl. In certain embodiments, the PEG can be substituted with methyl at the terminal hydroxyl position. In certain embodiments, the PEG can have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In certain embodiments, the terminal hydroxyl group can be substituted with a methoxy or methyl group.

PEG-modified lipids include the PEG-dialkyloxypropyl conjugates (PEG-DAA) described in U.S. Pat. Nos. 8,936,942 and 7,803,397, the disclosures of which are herein incorporated in their entirety. PEG-modified lipids (or lipid-polyoxyethylene conjugates) that are useful can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerCl4 or PEG-CerC20) which are described in U.S. Pat. No. 5,820,873, the disclosure of which is herein incorporated in its entirety, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.5% by weight of the LNP to about 20% by weight of the LNP, or from about 5% by weight of the LNP to about 15% by weight of the LNP.

Furthermore, LNP can be a PEG-modified and a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

In certain embodiments, prior to encapsulating LNPs can have a size in a range from about 10 nm to 500 nm, or from about 50 nm to about 200 nm, or from 75 nm to about 125 nm. In certain embodiments, LNP encapsulated nucleic acid, expression vector, AAV vector, or non-viral vector can have a size in a range from about 10 nm to 500 nm.

### Polymer-Based Systems

Polymer-based delivery systems are well known in the art, and any suitable polymer-based delivery system or polymeric nanoparticle known to those skilled in the art in view of the present disclosure can be used in the invention. DNA can be entrapped into the polymeric matrix of polymeric nanoparticles or can be adsorbed or conjugated on the surface of the nanoparticles. Examples of commonly used polymers for gene delivery include, e.g., poly(lactic-co-glycolic acid) (PLGA), poly lactic acid (PLA), poly(ethylene imine) (PEI), chitosan, dendrimers, polyanhydride, polycaprolactone, and polymethacrylates.

The polymeric-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Protein-Based Systems

Protein-based delivery systems are well known in the art, and any suitable protein-based delivery system or cell-penetrating peptide (CPP) known to those skilled in the art in view of the present disclosure can be used in the invention.

CPPs are short peptides (6-30 amino acid residues) that are potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25(1):1996-2006). Examples of CPPs include, e.g., cationic CPPs (highly positively charged) (e.g., the Tat peptide, penetratin, protamine, poly-L-lysine, polyarginine, etc.); amphipathic CPPs (chimeric or fused peptides, constructed from different sources, containing both positively and negatively charged amino acid sequences) (e.g., transportan, VT5, bactenecin-7 (Bac7), proline-rich peptide (PPR), SAP (VRLPPP)₃, TP10, pep-1, MPG, etc.); membranotropic CPPs (exhibit both hydrophobic and amphipathic nature simultaneously, and comprise both large aromatic residues and small residues) (e.g., gH625, SPIONs-PEG-CPP NPs, etc.); and hydrophobic CPPs (contain only non-polar motifs or residues) (e.g., SG3, PFVYLI, pep-7, fibroblast growth factors (FGF), etc.).

The protein-based non-viral vectors can have different sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Peptide Cage Systems

Peptide cage-based delivery systems are well known in the art, and any suitable peptide cage-based delivery system known to those skilled in the art in view of the present disclosure can be used in the invention. In general, any proteinaceous material that is able to be assembled into a cage-like structure, forming a constrained internal environment, can be used. Several different types of protein “shells” can be assembled and loaded with different types of materials. For example, protein cages comprising a shell of viral coat protein(s) (e.g., from the Cowpea Chlorotic Mottle Virus (CCMV) protein coat) that encapsulate a non-viral material, as well as protein cages formed from non-viral proteins have been described (see, e.g., U.S. Pat. Nos. 6,180,389 and 6,984,386, U.S. Patent Application 20040028694, and U.S. Patent Application 20090035389, the disclosures of which are herein incorporated in their entirety). Peptide cages can comprise a proteinaceous shell that self-assembles to form a protein cage (e.g., a structure with an interior cavity which is either naturally accessible to the solvent or can be made to be so by altering solvent concentration, pH, equilibria ratios).

Examples of protein cages derived from non-viral proteins include, e.g., ferritins and apoferritins, derived from both eukaryotic and prokaryotic species, e.g., 12 and 24 subunit ferritins; and protein cages formed from heat shock proteins (HSPs), e.g., the class of 24 subunit heat shock proteins that form an internal core space, the small HSP of Methanococcus jannaschii, the dodecameric Dsp HSP of E. coli, the MrgA protein, etc. As will be appreciated by those in the art, the monomers of the protein cages can be naturally occurring or variant forms, including amino acid substitutions, insertions and deletions (e.g., fragments) that can be made.

The protein cages can have different core sizes, ranging from about 1 nm to about 1000 nm, optionally from about 10 nm to about 500 nm, optionally from about 50 nm to about 200 nm, optionally about 100 nm to about 150 nm, optionally about 150 nm or less.

### Pharmaceutical Compositions

The invention additionally provides pharmaceutical compositions comprising any of the polynucleotides comprising the nucleic acids encoding GLA, expression cassettes comprising polynucleotides comprising the nucleic acids encoding GLA, viral vectors such as AAV vectors comprising polynucleotides comprising the nucleic acids encoding GLA, or non-viral vectors comprising polynucleotides comprising the nucleic acids encoding GLA as set forth herein.

rAAV vectors and non-viral vectors can be administered to a patient via infusion in a biologically compatible carrier, for example, via intravenous injection. rAAV vectors and non-viral vectors can be administered alone or in combination with other molecules. Accordingly, rAAV vectors and non-viral vectors and other compositions, agents, drugs, biologics (proteins) can be incorporated into pharmaceutical compositions. Such pharmaceutical compositions are useful for, among other things, administration and delivery to a subject in vivo or ex vivo.

In certain embodiments, pharmaceutical compositions also contain a pharmaceutically acceptable carrier or excipient. Such excipients include any pharmaceutical agent that does not itself induce an immune response harmful to the individual receiving the composition, and which can be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material can be administered to a subject without causing substantial undesirable biological effects. Thus, such a pharmaceutical composition can be used, for example in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Excipients also include proteins such as albumin.

Additionally, auxiliary substances, such as wetting or emulsifying agents, pH buffering substances, and the like, can be present in such vehicles.

The pharmaceutical composition can be provided as a salt and can be formed with different acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation can be a lyophilized powder which can contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions can include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions can contain substances which increase the viscosity of the suspension, such as sodium carboxymethyl cellulose, sorbitol, or dextran.

Additionally, suspensions of the active compounds can be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension can also contain suitable stabilizers or agents which increase the solubility of the compounds to allow for the preparation of highly concentrated solutions.

Cosolvents and adjuvants can be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

In certain embodiments, a pharmaceutical composition comprising any of the AAV vectors as set forth herein, further comprises empty AAV capsids. In certain embodiments, in a pharmaceutical composition comprising AAV vectors and empty AAV capsids, the ratio of the empty AAV capsids to the AAV vector is within or between about 100:1-50:1, from about 50:1-25:1, from about 25:1-10:1, from about 10: 1-1:1, from about 1:1-1:10, from about 1:10-1:25, from about 1:25-1:50, or from about 1:50-1:100. In particular aspects, in a pharmaceutical composition comprising AAV vectors and empty AAV capsids, the ratio of the of the empty AAV capsids to the AAV vector is about 2:1, 3:1, 4:1, 5:1, 6:1, 7:1, 8:1, 9:1, or 10:1.

In certain embodiments, a pharmaceutical composition includes a surfactant.

After pharmaceutical compositions have been prepared, they can be placed in an appropriate container and labeled for treatment. Such labeling could include amount, frequency, and method of administration.

Pharmaceutical compositions and delivery systems appropriate for the compositions, methods and uses of the invention are known in the art (see, e.g., Remington: The Science and Practice of Pharmacy (2003) 20th ed., Mack Publishing Co., Easton, PA; Remington's Pharmaceutical Sciences (1990) 18th ed., Mack Publishing Co., Easton, PA; The Merck Index (1996) 12th ed., Merck Publishing Group, Whitehouse, NJ; Pharmaceutical Principles of Solid Dosage Forms (1993), Technomic Publishing Co., Inc., Lancaster, Pa.; Ansel and Stoklosa, Pharmaceutical Calculations (2001) 11th ed., Lippincott Williams & Wilkins, Baltimore, MD; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosuppressive agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

Compositions such as pharmaceutical compositions can be delivered to a subject, so as to allow production of the encoded protein. In certain embodiments, pharmaceutical compositions comprise sufficient genetic material to enable a recipient to produce a therapeutically effective amount of a protein in the subject.

A “therapeutically effective amount” refers to an amount of an active ingredient or component that elicits the desired biological or medicinal response in a subject. A therapeutically effective amount can be determined empirically and in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration, and the severity of disease, and should be decided according to the judgment of the practitioner and each patient's circumstances. Effective doses can be extrapolated from dose-response curves derived from in vitro or animal model test systems.

Compositions can be formulated and/or administered in any sterile, biocompatible pharmaceutical carrier, including, but not limited to, saline, buffered saline, dextrose, and water. The compositions can be formulated and/or administered to a patient alone, or in combination with other agents (e.g., co-factors) which influence hemostasis.

### Methods of Treatment

The invention still further provides methods of treating a subject in need of GLA, comprising administering to the subject a therapeutically effective amount of a nucleic acid, expression cassette, AAV vector, non-viral vector, or pharmaceutical composition of the invention, wherein the GLA is expressed in the subject.

Methods and uses of the invention include delivering (transducing) nucleic acid (transgene) into host cells, including dividing and/or non-dividing cells. The polynucleotides, expression cassettes, rAAV vectors, non-viral vectors, methods, uses and pharmaceutical formulations of the invention are additionally useful in a method of delivering, administering or providing protein encoded by heterologous nucleic acid to a subject in need thereof, as a method of treatment. In this manner, the polynucleotide comprising the nucleic acid is transcribed and a protein produced in vivo in a subject. The subject can benefit from or be in need of the protein because the subject has a deficiency of the protein, or because production of the protein in the subject can impart some therapeutic effect, as a method of treatment or otherwise.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of protein/enzyme deficiencies such as Fabry disease, and lysosomal storage diseases and others known to those of skill in the art.

Subjects appropriate for treatment in accordance with the invention include those having or at risk of producing an insufficient amount of GLA, or producing an aberrant, partially functional or non-functional GLA. Subjects can be tested for GLA activity to determine if such subjects are appropriate for treatment according to a method of the invention. Subjects appropriate for treatment in accordance with the invention also include those subjects that would benefit from GLA. Such subjects that can benefit from GLA include those having a lysosomal storage disease. Treated subjects can be monitored after treatment periodically, e.g., every 1-4 weeks, 1-6 months, 6-12 months, or 1, 2, 3, 4, 5 or more years.

Subjects can be tested for an immune response, e.g., antibodies against AAV. Candidate subjects can therefore be screened prior to treatment according to a method of the invention. Subjects also can be tested for antibodies against AAV after treatment, and optionally monitored for a period of time after treatment. Subjects having pre-existing or developing AAV antibodies can be treated with an immunosuppressive agent, or other regimen as set forth herein.

Subjects appropriate for treatment in accordance with the invention also include those having or at risk of producing antibodies against AAV. rAAV vectors can be administered or delivered to such subjects using several techniques. For example, AAV empty capsid (i.e., AAV lacking a modified nucleic acid encoding GLA) can be delivered to bind to the AAV antibodies in the subject thereby allowing the rAAV vector comprising the heterologous nucleic acid to transduce cells of the subject.

The modified nucleic acids, expression cassettes, rAAV vectors, and non-viral vectors of the invention can be used for treatment of a GLA deficiency. Accordingly, in certain embodiments, modified nucleic acids encoding GLA, expression cassettes comprising modified nucleic acids encoding GLA, rAAV vectors, and non-viral vectors of the invention can be used as a therapeutic and/or prophylactic agent.

In certain embodiments, the modified nucleic acids encoding GLA, expression cassettes comprising modified nucleic acids encoding GLA, rAAV vectors, and non-viral vectors of the invention can be used for treatment of Fabry disease. Administration of modified nucleic acids encoding GLA, expression cassettes comprising modified nucleic acids encoding GLA, rAAV vectors, and non-viral vectors of the invention to a patient with Fabry or another lysosomal storage disease leads to the expression of the GLA protein.

In certain embodiments, a method according to the instant invention can result in expression or activity of GLA at a level that is at least 1%, at least 5%, at least 10%, at least 15%, at least 20%, at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, at least 100% of normal expression of the GLA protein found in a subject not in need of GLA.

In certain embodiments, a method according to the instant invention can result in expression or activity of GLA in the kidney at a level that is at least 1%, at least 5%, at least 10%, at least 15%, at least 20%, at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, at least 100% of normal expression of the GLA protein found in the kidney of a subject not in need of GLA.

In certain embodiments, a method according to the instant invention can result in expression or activity of GLA in the heart at a level that is at least 1%, at least 5%, at least 10%, at least 15%, at least 20%, at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, at least 100% of normal expression of the GLA protein found in the heart of a subject not in need of GLA.

In certain embodiments, a method according to the instant invention can result in expression or activity of GLA in the liver at a level that is at least 1%, at least 5%, at least 10%, at least 15%, at least 20%, at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, at least 100% of normal expression of the GLA protein found in the liver of a subject not in need of GLA.

In certain embodiments, a method according to the instant invention can result in expression or activity of GLA in the bloodstream at a level that is at least 1%, at least 5%, at least 10%, at least 15%, at least 20%, at least 25%, at least 30%, at least 35%, at least 40%, at least 45%, or at least 50%, at least 55%, at least 60%, at least 65%, at least 70%, at least 75%, at least 80%, at least 85%, at least 90%, at least 95%, at least 100% of normal expression of the GLA protein found in the bloodstream of a subject not in need of GLA.

Subjects, animals or patients administered the modified nucleic acids encoding GLA, expression cassettes comprising modified nucleic acids encoding GLA, rAAV vectors, and non-viral vectors of the invention can be evaluated by a variety of tests, assays and functional assessments to demonstrate, measure and/or assess efficacy of the modified nucleic acids encoding GLA, expression cassettes comprising modified nucleic acids encoding GLA, rAAV vectors, and non-viral vectors of the invention as therapeutic and/or prophylactic agents.

Such tests and assays include, but are not limited to, measurement of GLA activity (such as by use of standard GLA activity assays) and or GLA amount (such as by western blot with anti-GLA antibody, or by ELISA quantification) in a biological sample such as blood, plasma, or urine (see, e.g., Christensen, E. et al., J Am Soc Nephrol. 2007 March; 18(3):698-706); analysis of peak and steady-state vector-derived GLA enzyme levels assessed by total GLA protein and activity in plasma; analysis of GLA enzyme levels and cross-correction assessed by total GLA protein and activity in tissue by immunofluorescence and immunohistochemistry (see, e.g., Christensen, E. et al., 2007, Id.); measurement of GLA substrate accumulation of globotriaosylceramide (Gb3 or GL3) and globotriaosylsphingosine (lyso-Gb3 or lyso-GL3) in tissue and serum (such as by quantitative liquid chromatography tandem mass spectrometry or by lipid analysis with thin layer chromatography (see, e.g., Shu et al., J Biol Chem. 2007 Jul. 20; 282(29):20960-7, and Shu et al., J Glycomics Lipidomics. 2012; Suppl 2:1-6); measurement of expression levels of Gb3 by immunofluorescence staining or electron microscopy (see, e.g., Braun et al., Cell Physiol Biochem. 2019; 52(5):1139-1150); testing for GLA mRNA by quantitative reverse transcriptase PCR in AAV-transduced tissue; testing for immune responses against AAV capsid; testing for immune responses against the GLA transgene protein product.

Additionally, the modified nucleic acids encoding GLA, expression cassettes comprising modified nucleic acids encoding GLA, rAAV vectors, and non-viral vectors of the invention can be used for treatment of a lysosomal storage disease. Lysosomal storage diseases include any disorder characterized by reduced or absent lysosomal enzyme activity. According to certain embodiments, the modified nucleic acids encoding GLA, expression cassettes comprising modified nucleic acids encoding GLA, rAAV vectors, and non-viral vectors of the invention can be used for treatment of a patient in need of GLA. According to certain embodiments, the modified nucleic acids encoding GLA, expression cassettes comprising modified nucleic acids encoding GLA, rAAV vectors, and non-viral vectors of the invention can be used for treatment of Fabry disease. According to certain embodiments, the modified nucleic acids encoding GLA, expression cassettes comprising modified nucleic acids encoding GLA, rAAV vectors, and non-viral vectors of the invention can be used to decrease the level of glycosphingolipids in the tissues of a subject.

As set forth herein, rAAV are useful as gene therapy vectors as they can penetrate cells and introduce nucleic acid/genetic material into the cells. Because AAV are not associated with pathogenic disease in humans, rAAV vectors are able to deliver heterologous polynucleotide sequences (e.g., therapeutic proteins and agents) to human patients without causing substantial AAV pathogenesis or disease.

rAAV vectors possess a number of desirable features for such applications, including tropism for dividing and non-dividing cells. Early clinical experience with these vectors also demonstrated no sustained toxicity and immune responses are typically minimal or undetectable. AAV are known to infect a wide variety of cell types in vivo by receptor-mediated endocytosis or by transcytosis. These vector systems have been tested in humans targeting many tissues, such as, retinal epithelium, liver, skeletal muscle, airways, brain, joints and hematopoietic stem cells.

It can be desirable to introduce a rAAV vector that can provide, for example, multiple copies of GLA and hence greater amounts of GLA protein. Improved rAAV vectors and methods for producing these vectors have been described in detail in a number of references, patents, and patent applications, including: Wright J. F. (Hum. Gene Ther., 20:698-706, 2009).

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration can be proportionally increased or reduced, as indicated by any adverse side effects, complications or other risk factors of the treatment or therapy and the status of the subject. The skilled artisan will appreciate the factors that can influence the dosage and timing required to provide an amount sufficient for providing a therapeutic or prophylactic benefit.

The dose to achieve a therapeutic effect, e.g., the dose in vector genomes/per kilogram of body weight (vg/kg) of rAAV, or the dose of non-viral vector, will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can determine a rAAV/vector genome or non-viral vector dose range to treat a patient having a particular disease or disorder based on the aforementioned factors, as well as other factors.

Generally, doses of rAAVs will range from at least 1×10⁸ vector genomes per kilogram (vg/kg) of the weight of the subject, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect.

For example, a dose of about 5×10¹¹ recombinant AAV vg/kg or greater than about 5×10¹¹ recombinant AAV vg/kg; a dose of about 1×10¹² recombinant AAV vg/kg or greater than about 1×10¹² recombinant AAV vg/kg; a dose of about 2×10¹² recombinant AAV vg/kg or greater than about 2×10¹² recombinant AAV vg/kg; a dose of about 3×10¹² recombinant AAV vg/kg or greater than about 3×10¹² recombinant AAV vg/kg; a dose of about 4×10¹² recombinant AAV vg/kg or greater than about 4×10¹² recombinant AAV vg/kg; a dose of about 5×10¹² recombinant AAV vg/kg or greater than about 5×10¹² recombinant AAV vg/kg; a dose of about 1×10¹³ recombinant AAV vg/kg or greater than about 1×10¹³ recombinant AAV vg/kg; a dose of about 2×10¹³ recombinant AAV vg/kg or greater than about 2×10¹³ recombinant AAV vg/kg; a dose of about 3×10¹³ recombinant AAV vg/kg or greater than about 3×10¹³ recombinant AAV vg/kg; a dose of about 4×10¹³ recombinant AAV vg/kg or greater than about 4×10¹³ recombinant AAV vg/kg; a dose of about 5×10¹³ recombinant AAV vg/kg or greater than about 5×10¹³ recombinant AAV vg/kg; a dose of about 6×10¹³ recombinant AAV vg/kg or greater than about 6×10¹³ recombinant AAV vg/kg.

Exemplary dose ranges of recombinant AAV vg/kg administered are a dose range from about 5×10¹¹ to about 6×10¹³ recombinant AAV vg/kg; a dose range from about 5×10¹¹ to about 5.5×10¹¹ recombinant AAV vg/kg; a dose range from about 5.5×10¹¹ to about 6×10¹¹ recombinant AAV vg/kg; a dose range from about 6×10¹¹ to about 6.5×10¹¹ recombinant AAV vg/kg; a dose range from about 6.5×10¹¹ to about 7×10¹¹ recombinant AAV vg/kg; a dose range from about 7×10¹¹ to about 7.5×10¹¹ recombinant AAV vg/kg; a dose range from about 7.5×10¹¹ to about 8×10¹¹ recombinant AAV vg/kg; a dose range from about 8×10¹¹ to about 8.5×10¹¹ recombinant AAV vg/kg; a dose range from about 8.5×10¹¹ to about 9×10¹¹ recombinant AAV vg/kg; a dose range from about 9×10¹¹ to about 9.5×10¹¹ recombinant AAV vg/kg; a dose range from about 9.5×10¹¹ to about 1×10¹² recombinant AAV vg/kg; a dose range from about 1×10¹² to about 1.5×10¹² recombinant AAV vg/kg; a dose range from about 1.5×10¹² to about 2×10¹² recombinant AAV vg/kg; a dose range from about 2×10¹² to about 2.5×10¹² recombinant AAV vg/kg; a dose range from about 2.5×10¹² to about 3×10¹² recombinant AAV vg/kg; a dose range from about 3×10¹² to about 3.5×10¹² recombinant AAV vg/kg; a dose range from about 3.5×10¹² to about 4×10¹² recombinant AAV vg/kg; a dose range from about 4×10¹² to about 4.5×10¹² recombinant AAV vg/kg; a dose range from about 4.5×10¹² to about 5×10¹² recombinant AAV vg/kg; a dose range from about 5×10¹² to about 5.5×10¹² recombinant AAV vg/kg; a dose range from about 5.5×10¹² to about 6×10¹² recombinant AAV vg/kg; a dose range from about 6×10¹² to about 6.5×10¹² recombinant AAV vg/kg; a dose range from about 6.5×10¹² to about 7×10¹² recombinant AAV vg/kg; a dose range from about 7×10¹² to about 7.5×10¹² recombinant AAV vg/kg; a dose range from about 7.5×10¹² to about 8×10¹² recombinant AAV vg/kg; a dose range from about 8×10¹² to about 8.5×10¹² recombinant AAV vg/kg; a dose range from about 8.5×10¹² to about 9×10¹² recombinant AAV vg/kg; a dose range from about 9×10¹² to about 9.5×10¹² recombinant AAV vg/kg; a dose range from about 9.5×10¹² to about 1×10¹³ recombinant AAV vg/kg; a dose range from about 1×10¹³ to about 1.5×10¹³ recombinant AAV vg/kg; a dose range from about 1.5×10¹³ to about 2×10¹³ recombinant AAV vg/kg; a dose range from about 2×10¹³ to about 2.5×10¹³ recombinant AAV vg/kg; a dose range from about 2.5×10¹³ to about 3×10¹³ recombinant AAV vg/kg; a dose range from about 3×10¹³ to about 3.5×10¹³ recombinant AAV vg/kg; a dose range from about 3.5×10¹³ to about 4×10¹³ recombinant AAV vg/kg; a dose range from about 4×10¹³ to about 4.5×10¹³ recombinant AAV vg/kg; a dose range from about 4.5×10¹³ to about 5×10¹³ recombinant AAV vg/kg; a dose range from about 5×10¹³ to about 5.5×10¹³ recombinant AAV vg/kg; a dose range from about 5.5×10¹³ to about 6×10¹³ recombinant AAV vg/kg; a dose range from about 6×10¹³ to about 1×10¹⁴ recombinant AAV vg/kg.

In certain embodiments, AAV vg/kg are administered at a dose of about 5×10¹¹ vg/kg, administered at a dose of about 6×10¹¹ vg/kg, administered at a dose of about 7×10¹¹ vg/kg, administered at a dose of about 8×10¹¹ vg/kg, administered at a dose of about 9×10¹¹ vg/kg, administered at a dose of about 1×10¹² vg/kg, administered at a dose of about 2×10¹² vg/kg, administered at a dose of about 3×10¹² vg/kg, administered at a dose of about 4×10¹² vg/kg, administered at a dose of about 5×10¹² vg/kg, administered at a dose of about 6×10¹² vg/kg, administered at a dose of about 7×10¹² vg/kg, administered at a dose of about 8×10¹² vg/kg, administered at a dose of about 9×10¹² vg/kg, administered at a dose of about 1×10¹³ vg/kg, administered at a dose of about 2×10¹³ vg/kg, administered at a dose of about 3×10¹³ vg/kg, administered at a dose of about 4×10¹³ vg/kg, administered at a dose of about 5×10¹³ vg/kg, administered at a dose of about 6×10¹³ vg/kg.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms can be within, for example, ampules and vials, which can include a liquid composition, or a composition in a freeze-dried or lyophilized state; a sterile liquid carrier, for example, can be added prior to administration or delivery in vivo. Individual unit dosage forms can be included in multi-dose kits or containers. rAAV particles, non-viral vectors, and pharmaceutical compositions thereof can be packaged in single or multiple unit dosage form for ease of administration and uniformity of dosage.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

In certain embodiments, a method according to the instant invention reduces, decreases or inhibits one or more symptoms of the need for GLA or of Fabry disease; or prevents or reduces progression or worsening of one or more symptoms of the need for GLA or of Fabry disease; or stabilizes one or more symptoms of the need for GLA or of Fabry disease; or improves one or more symptoms of the need for GLA or of Fabry disease.

An effective amount or a sufficient amount can but need not be provided in a single administration, can require multiple administrations, and, can but need not be, administered alone or in combination with another composition (e.g., agent), treatment, protocol or therapeutic regimen. For example, the amount can be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens can be included in order to be considered effective or sufficient in a given subject. Amounts considered effective also include amounts that result in a reduction of the use of another treatment, therapeutic regimen or protocol, such as administration of modified nucleic acid encoding GLA for treatment of a GLA deficiency (e.g., Fabry disease) or another lysosomal storage disease that can be treated with GLA.

Accordingly, methods and uses of the invention also include, among other things, methods and uses that result in a reduced need or use of another compound, agent, drug, therapeutic regimen, treatment protocol, process, or remedy. For example, for GLA deficiency, a method or use of the invention has a therapeutic benefit if in a given subject, a less frequent or reduced dose or elimination of administration of a recombinant GLA to supplement for the deficient or defective GLA in the subject is needed. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (e.g., GLA or a protein deficiency that leads to a lysosomal storage disease that can be treated with GLA), or that produce an aberrant, partially functional or non-functional gene product (e.g., GLA or a protein implicated in a lysosomal storage disease that can be treated with GLA).

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-24, 24-48, 48-64 or more days, months or years after a subject has been identified as having the disease targeted for treatment, has one or more symptoms of the disease, or has been screened and is identified as positive as set forth herein.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

For Fabry disease, an effective amount would be an amount that improves markers for Fabry disease, such as globotriaosylsphingosine (lyso-GB3) and those disclosed in US Patent Application Publication No. US 2010-0113517, the disclosure of which is herein incorporated in its entirety. Non-limiting examples of improvements in surrogate markers for Fabry disease disclosed in US 2010/0113517 include increases in α-Gal A levels or activity in cells (e.g., fibroblasts) and tissue; reductions in GL-3 accumulation; decreased plasma concentrations of homocysteine and vascular cell adhesion molecule-1 (VCAM-1); decreased GL-3 accumulation within myocardial cells and valvular fibrocytes; reduction in cardiac hypertrophy (especially of the left ventricle), amelioration of valvular insufficiency, and arrhythmias; amelioration of proteinuria; decreased urinary concentrations of lipids such as CTH, lactosylceramide, ceramide, and increased urinary concentrations of glucosylceramide and sphingomyelin; the absence of laminated inclusion bodies (Zebra bodies) in glomerular epithelial cells; improvements in renal function; mitigation of hypohidrosis; the absence of angiokeratomas; and improvements in hearing abnormalities such as high frequency sensorineural hearing loss, progressive hearing loss, sudden deafness, or tinnitus.

Improvements in neurological symptoms include prevention of transient ischemic attack (TIA) or stroke; and amelioration of neuropathic pain manifesting itself as acroparaesthesia (burning or tingling in extremities). Another type of clinical marker that can be assessed for Fabry disease is the prevalence of deleterious cardiovascular manifestations. Common cardiac-related signs and symptoms of Fabry disease include left ventricular hypertrophy, valvular disease (especially mitral valve prolapse and/or regurgitation), premature coronary artery disease, angina, myocardial infarction, conduction abnormalities, arrhythmias, congestive heart failure.

Therapeutic doses will depend on, among other factors, the age and general condition of the subject, the severity of the disease or disorder. Thus, a therapeutically effective amount in humans will fall in a relatively broad range that can be determined by a medical practitioner based on the response of an individual patient.

In additional embodiments, an effective amount administered to a human subject provides: an increase of plasma GLA to greater than 1 ng/ml, greater than 2 ng/ml, greater 3 ng/ml, greater than 4 ng/ml, about 1 ng/ml, about 2 ng/ml, about 2.5 ng/ml, about 3 ng/ml, or about 3.5 ng/ml; an increase in plasma GLA activity to greater than 1 nmol/h/mL, greater than 1.5 nmol/h/mL, greater than 2 nmol/h/mL, greater than 2.5 nmol/h/mL, greater than 3 nmol/h/mL, greater than 4 nmol/h/mL, greater than 5 nmol/h/mL, greater than 6 nmol/h/mL, greater than 7 nmol/h/mL, about 1 nmol/h/mL, about 1.5 nmol/h/mL, about 2 nmol/h/mL, about 2.5 nmol/h/mL, about 3 nmol/h/mL, about 4 nmol/h/mL, about 5 nmol/h/mL, about 6 nmol/h/m, or about 7 nmol/h/mL; and/or a decrease of plasma Lyso-Gb3 to less than 40 nmol/L, less than 30 nmole/L, less than 10 nmole/L, less than 5 nmole/L, or less than 2 nmole/L. Plasma GLA, plasma GLA activity and plasma Lyso-Gb3 can be measured using standard techniques such as those provided, or referenced, in Tsukimura et al., Molecular Genetics and Metabolism Reports 1 (2014) 288-298.

Methods and uses of the invention include delivery and administration systemically, regionally or locally, or by any route, for example, by injection or infusion. Delivery of the pharmaceutical compositions in vivo can generally be accomplished via injection using a conventional syringe, although other delivery methods such as convection-enhanced delivery are envisioned (See e.g., U.S. Pat. No. 5,720,720, the disclosure of which is herein incorporated in its entirety). For example, compositions can be delivered subcutaneously, epidermally, intradermally, intrathecally, intraorbitally, intramucosally, intranasally, intraperitoneally, intravenously, intra-pleurally, intraarterially, intracavitary, orally, intrahepatically, via the portal vein, or intramuscularly. Other modes of administration include oral and pulmonary administration, suppositories, and transdermal applications. A clinician specializing in the treatment of patients with Fabry or other lysosomal storage diseases can determine the optimal route for administration of AAV vectors and non-viral vectors based on a number of criteria, including, but not limited to: the condition of the patient and the purpose of the treatment.

The compositions can be administered alone. In certain embodiments, an rAAV particle or a non-viral vector provides a therapeutic effect without an immunosuppressive agent. The therapeutic effect optionally is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more without administering an immunosuppressive agent. Accordingly, a therapeutic effect is provided for a period of time.

rAAV vectors, non-viral vectors, methods, and uses of the invention can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method or use of the invention.

The compound, agent, drug, treatment or other therapeutic regimen or protocol can be administered as a combination composition, or administered separately, such as concurrently or in series or sequentially (prior to or following) to delivery or administration of a nucleic acid, expression cassette, rAAV particle, or non-viral vector. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, expression cassette, non-viral vector, or rAAV particle of the invention, to a subject.

In certain embodiments, nucleic acids, expression vectors, non-viral vectors, or rAAV particles of the invention are administered to a patient in combination with an immunosuppressive agent or regimen where the patient has or is at risk of developing an immune response against the rAAV particle and/or the GLA protein. Such immunosuppressive agent or regimen can be administered prior to, substantially at the same time or after administering a nucleic acid, expression cassette, non-viral vector, or rAAV vector of the invention.

In certain embodiments, a subject or patient, such as a human patient, with Fabry disease has developed inhibitors to the GLA protein (including anti-GLA antibodies and/or anti-GLA T-cells), which can occur following treatment with traditional enzyme replacement therapy (e.g., following administration of recombinantly produced GLA protein). The development of such GLA inhibitors can occur in patients that receive enzyme replacement therapy, particularly where the patient has undetectable GLA levels (as can be the case in infantile Fabry disease), leading the patient's immune system to see the replacement GLA protein as “foreign.” In certain embodiments, a Fabry patient having GLA inhibitors is administered one or more regimen intended to achieve immune tolerance or mitigate the immune response to the GLA protein in the patient, prior to, substantially at the same time or after administering an rAAV vector or non-viral vector of the invention. Such regimens to achieve immune tolerance or mitigate the immune response to the GLA protein can include administration of one or more immunosuppressive agent, including but not limited to methotrexate, rituximab, intravenous gamma globulin (IVIG), omalizumab, and synthetic vaccine particle (SVP™)-rapamycin (rapamycin encapsulated in a biodegradable nanoparticle) and/or administration of one or more immunosuppressive protocol or procedure, such as B-cell depletion, immunoadsorption, and plasmapheresis.

In certain embodiments, rAAV vector or non-viral vector is administered in conjunction with one or more immunosuppressive agents prior to, substantially at the same time or after administering an rAAV vector or a non-viral vector. In certain embodiments, the one or more immunosuppressive agents is administered, e.g., 1-12, 12-24 or 24-48 hours, or 2-4, 4-6, 6-8, 8-10, 10-14, 14-20, 20-25, 25-30, 30-50, or more than 50 days following administering an rAAV vector or a non-viral vector. Such administration of immunosuppressive agents after a period of time following administering rAAV vector or non-viral vector can be done if there is a decrease in the encoded protein or inhibitory nucleic acid after the initial expression levels for a period of time, e.g., 20-25, 25-30, 30-50, 50-75, 75-100, 100-150, 150-200 or more than 200 days following rAAV vector or non-viral vector.

In certain embodiments, an immunosuppressive agent is an anti-inflammatory agent.

In certain embodiments, an immunosuppressive agent is a steroid, e.g., a corticosteroid. In certain embodiments, an immunosuppressive agent is prednisone, prednisolone, calcineurin inhibitor (e.g., cyclosporine, tacrolimus), MMF (mycophenolic acid, e.g. CellCept®, Myfortic®), CD52 inhibitor (e.g., alemtuzumab), CTLA4-Ig (e.g., abatacept, belatacept), anti-CD3 mAb, anti-LFA-1 mAb (e.g., efalizumab), anti-CD40 mAb (e.g., ASKP1240), anti-CD22 mAb (e.g., epratuzumab), anti-CD20 mAb (e.g., rituximab, orelizumab, ofatumumab, veltuzumab), proteasome inhibitor (e.g., bortezomib), TACI-Ig (e.g., atacicept), anti-C5 mAb (e.g., eculizumab), mycophenolate, azathioprine, sirolimus everolimus, TNFR-Ig, anti-TNF mAb, tofacitinib, anti-IL-2R (e.g., basiliximab), anti-IL-17 mAb (e.g., secukinumab), anti-IL-6 mAb (e.g., anti-IL-6 antibody sirukumab, anti-IL-6 receptor antibody tocilizumab (Actemra®), IL-10 inhibitor, TGF-beta inhibitor, a B cell targeting antibody (e.g., rituximab), a mammalian target of rapamycin (mTOR) inhibitor (e.g., rapamycin), synthetic vaccine particle (SVP™)-rapamycin (rapamycin encapsulated in a biodegradable nanoparticle), intravenous gamma globulin (IVIG), omalizumab, methotrexate, a tyrosine kinase inhibitor (e.g., ibrutinib), cyclophosphamide, fingolimod, an inhibitor of B-cell activating factor (BAFF) (e.g, anti-BAFF mAb, e.g., belimumab), an inhibitor of a proliferation-inducing ligand (APRIL), anti-IL-1b mAb (e.g., canakinumab (Haris®)), a C3a inhibitor, a Tregitope (see, e.g., U.S. Pat. No. 10,213,496), or a combination and/or derivative thereof, and/or administration of one or more immunosuppressive protocol or procedure, such as B-cell depletion, immunoadsorption, and plasmapheresis.

Immune-suppression protocols, including the use of rapamycin, alone or in combination with IL-10, can be used to decrease, reduce, inhibit, prevent or block humoral and cellular immune responses to the GLA protein. Hepatic gene transfer with AAV vectors of the invention can be used to induce immune tolerance to the GLA protein through induction of regulatory T cells (Tregs) and other mechanisms. Strategies to reduce (overcome) or avoid humoral immunity to AAV in systemic gene transfer include, administering high vector doses, use of AAV empty capsids as decoys to adsorb anti-AAV antibodies, administration of immunosuppressive drugs to decrease, reduce, inhibit, prevent or eradicate the humoral immune response to AAV, changing the AAV capsid serotype or engineering the AAV capsid to be less susceptible to neutralizing antibodies, use of plasma exchange cycles to adsorb anti-AAV immunoglobulins, thereby reducing anti-AAV antibody titer, and use of delivery techniques such as balloon catheters followed by saline flushing. Such strategies are described in Mingozzi et al., 2013, Blood, 122:23-36. Additional strategies include use of AAV-specific plasmapheresis columns to selectively deplete anti-AAV antibodies without depleting the total immunoglobulin pool from plasma, as described in Bertin et al., 2020, Sci. Rep. 10:864. https://doi.org/10.1038/s41598-020-57893-z.

Ratio of AAV empty capsids to the rAAV vector can be, for example, within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100. Ratios can also be about 2:1, 3:1, 4:1, 5:1, 6:1, 7:1, 8:1, 9:1, or 10:1.

Amounts of AAV empty capsids to administer can be calibrated based upon the amount (titer) of AAV antibodies produced in a particular subject. AAV empty capsids can be of any serotype, for example, AAV1, AAV2, AAV3, AAV4, AAV5, AAV6, AAV7, AAV8, AAV9, AAV10, AAV 11, AAV12, Rh10, Rh74 (SEQ ID NO: 35), AAV3B, LK03 (SEQ ID NO: 42) AAV-2i8, the sequence of SEQ ID NO: 110, the sequence of SEQ ID NO: 36, and/or the sequence of SEQ ID NO: 37.

Alternatively, or in addition, rAAV vector or non-viral vector can be delivered by direct intramuscular injection (e.g., one or more slow-twitch fibers of a muscle). In another alternative, a catheter introduced into the femoral artery can be used to deliver rAAV vectors or non-viral vectors to liver via the hepatic artery. Non-surgical means can also be employed, such as endoscopic retrograde cholangiopancreatography (ERCP), to deliver rAAV vectors or non-viral vectors directly to the liver, thereby bypassing the bloodstream and AAV antibodies. Other ductal systems, such as the ducts of the submandibular gland, can also be used as portals for delivering rAAV vectors or non-viral vectors into a subject that develops or has preexisting anti-AAV antibodies.

Additional strategies to reduce humoral immunity to AAV include methods to remove, deplete, capture, and/or inactivate AAV antibodies, commonly referred to as apheresis and more particularly, plasmapheresis where blood products are involved. Apheresis or plasmapheresis, is a process in which a human subject's plasma is circulated ex vivo (extracorporal) through a device that modifies the plasma through addition, removal and/or replacement of components before its return to the patient. Plasmapheresis can be used to remove human immunoglobulins (e.g., IgG, IgE, IgA, IgD) from a blood product (e.g., plasma). This procedure depletes, captures, inactivates, reduces or removes immunoglobulins (antibodies) that bind AAV thereby reducing the titer of AAV antibodies in the treated subject that can contribute to AAV vector neutralization. An example is a device composed of an AAV capsid affinity matrix column. Passing blood product (e.g., plasma) through an AAV capsid affinity matrix would result in binding only of AAV antibodies, and of all isotypes (including IgG, IgM, etc.).

A sufficient amount of plasmapheresis using an AAV capsid affinity matrix is predicted to substantially remove AAV capsid antibodies, and reduce the AAV capsid antibody titer (load) in the human. In certain embodiments, titer in a treated subject is reduced substantially to low levels (to <1:5, or less, such as <1:4, or <1:3, or <1:2, or <1:1).

A reduction in antibody titer will be temporary because the B lymphocytes that produce the AAV capsid antibodies would be expected to gradually cause the AAV capsid antibody titer to rebound to the steady state level prior to plasmapheresis.

In the case where a pre-existing AAV antibody titer was reduced from 1:100 to 1:1, AAV antibody titer rebounds of approximately 0.15% (corresponding to a titer of 1:1.2) 0.43% (1:1.4), 0.9% (1:1.9), 1.7% (1:2.7), and 3.4% (1:4.4), occur at 1 hour, 3 hours, 6 hours, 12 hours and 24 hours, respectively, after completion of the plasmapheresis method.

Temporary removal of AAV antibodies from such a subject would correspond to a window of time (for example, of about 24 hours or less, such as 12 hours or less, or 6 hours or less, or 3 hours or less, or 2 hours or less, or 1 hour or less) during which an AAV vector could be administered to the subject and predicted to efficiently transduce target tissues without substantial neutralization of the AAV vector with the AAV antibodies.

In the case where a pre-existing AAV antibody titer was reduced from 1:1000 to 1:1, AAV antibody titer rebounds of approximately 0.15% (corresponding to a titer of 1:2.5) 0.4% (1:5.3), 0.9% (1:9.7), 1.7% (1:18), and 3.4% (1:35), occur at 1 hour, 3 hours, 6 hours, 12 hours and 24 hours, respectively, after completion of the plasmapheresis method. Thus, a window for administration of AAV vector will be comparatively shorter.

AAV antibodies can be preexisting and can be present at levels that reduce or block therapeutic GLA gene transfer vector transduction of target cells. Alternatively, AAV antibodies can develop after exposure to AAV or administration of an AAV vector. If such antibodies develop after administration of an AAV vector, these subjects can also be treated via apheresis, more particularly, plasmapheresis.

In certain embodiments, the polynucleotides, expression cassettes, AAV vectors, and non-viral vectors of the invention can be used in combination with methods to reduce antibody (e.g., IgG) levels in human plasma. In certain embodiments, the polynucleotides, expression cassettes, AAV vectors, and non-viral vectors of the invention can be used in combination with an agent that that blocks, inhibits, or reduces the interaction of IgG with the neonatal Fc receptor (FcRn), such as an anti-FcRn antibody, to reduce IgG recycling and enhance IgG clearance in vivo, and/or an agent that decreases the circulating antibodies that bind to a viral vector, such as a recombinant viral vector, or that bind to a nucleic acid or a polypeptide, protein or peptide encoded by a therapeutic heterologous polynucleotide encapsidated by a recombinant viral vector, or that bind to the therapeutic heterologous polynucleotide.

In certain embodiments, antibody binding to a viral vector is reduced or inhibited by way of an agent that reduces interaction of IgG with FcRn, a protease or a glycosidase.

In certain embodiments, the polypeptides, expression cassettes, AAV vectors, or non-viral vectors of the invention can be used in combination with an endopeptidase (e.g., IdeS from Streptococcus pyogenes) or a modified variant thereof, or an endoglycosidase (e.g., S. pyogenes EndoS) or a modified variant thereof. In certain embodiments polypeptides, expression cassettes, AAV vectors, or non-viral vectors of the invention are administered to a subject in combination with an endopeptidase (e.g., IdeS from Streptococcus pyogenes) or a modified variant thereof, or an endoglycosidase (e.g., EndoS from S. pyogenes) or a modified variant thereof to reduce or clear neutralizing antibodies against AAV capsid and enable treatment of patients previously viewed as not eligible for gene therapy or that develop AAV antibodies after AAV gene therapy. Such strategies are described in Leborgne et al., 2020, Nat. Med., 26:1096-1101 (2020).

In certain embodiments, the nucleic acids, expression cassettes, AAV vectors, and non-viral vectors of the invention can be used in combination with symptomatic and support therapies, including, for example, bronchodilators; hearing aids; topical skin moisturizers; typical cardiac treatments such as diuretics, ACE inhibitors, cardiac devices, etc.; medications for pain relief or nephroprotection; stroke prophylaxis with antithrombotic and antiarrhythmic therapies; antiproteinuric agents, renal dialysis and/or kidney transplantation in the case of end stage renal failure; and metoclopramide, H2 blockers, and dietary therapy to ensure proper nutrition and manage gastrointestinal symptoms (see, e.g., Germain, Orphanet J Rare Dis. 2010 Nov. 22; 5:30; Ortiz et al., Mol Genet Metab. 2018 April; 123(4):416-427; and Mehta et al., QJM: Inter. Jour. Med. 2010 September; 103(9):641-659).

In certain embodiments, the polynucleotides, expression cassettes and AAV vectors of the invention can be used in combination with pharmacological chaperone therapy (also known as enzyme enhancement therapy), where one or more pharmacological chaperones is administered before, concomitant with, or after administration of the polynucleotide, expression cassette, AAV vector, or non-viral vectors of the invention, for the treatment of a lysosomal storage disease, such as Fabry disease.

In certain embodiments, the polynucleotides, expression cassettes, AAV vectors, and non-viral vectors of the invention can be used in combination with one or more pharmacological chaperone, which can stabilize GLA protein. Pharmacological chaperones that can be used in combination with the polynucleotides, expression cassettes and AAV vectors of the invention include, e.g., 1-deoxygalactonojirimycin (DGJ), migalastat hydrochloride (Migalastat), α-3,4-di-epi-homonojirimycin, 4-epi-fagomine, α-allo-homonojirimycin, N-methyl-deoxygalactonojirimycin, β-1-C-butyl-deoxygalactonojirimycin, α-galacto-homonojirimycin, calystegine A3, calystegine B2, calystegine B3, N-methyl-calystegine A3, N-methyl-calystegine B2 and N-methyl-calystegine B3, and others as described in U.S. Pat. Nos. 6,274,597, 6,774,135, and 6,599,919, the disclosures of which are herein incorporated in their entirety.

In certain embodiments, the polynucleotides and expression cassettes of the invention are delivered or administered via AAV vector particles. In certain embodiments, the polynucleotides and expression cassettes of the invention can be delivered or administered via other types of viral particles, including retroviral, adenoviral, helper-dependent adenoviral, hybrid adenoviral, herpes simplex virus, lentiviral, poxvirus, Epstein-Barr virus, vaccinia virus, and human cytomegalovirus particles. In certain embodiments, the polynucleotides and expression cassettes of the invention can be delivered or administered via non-viral vectors.

### Kits

The invention provides kits with packaging material and one or more components therein. A kit typically includes a label or packaging insert including a description of the components or instructions for use in vitro, in vivo, or ex vivo, of the components therein. A kit can contain a collection of such components, e.g., a rAAV particle or a non-viral vector, and optionally a second active, such as another compound, agent, drug or composition.

A kit refers to a physical structure housing one or more components of the kit.

Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacture location and date, expiration dates. Labels or inserts can include information on a disease for which a kit component can be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component can provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that can be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

TABLE 1
Sequence Table

| | |
|---|---|
| Sp7 | MAFLWLLSCWALLGTTFG (SEQ ID NO: 41) |
| Sp7_1 | ATGGCCTTCCTGTGGCTGCTGTCCTGCTGGGCACTGCTGGGCACCACA |
| | TTTGGC (SEQ ID NO: 1) |
| Sp7_2 | ATGGCCTTTCTGTGGCTGCTGTCCTGCTGGGCCCTGCTGGGGACCACC |
| | TTTGGC (SEQ ID NO: 2) |
| Sp7_3 | ATGGCCTTCCTGTGGCTGCTGAGCTGCTGGGCCCTGCTGGGGACCAC |
| | CTTTGGG (SEQ ID NO: 3) |
| Sp7_4 | ATGGCCTTCCTGTGGCTCCTGAGCTGCTGGGCCCTCCTGGGGACCACC |
| | TTTGGG (SEQ ID NO: 4) |
| Sp7_5 | ATGGCCTTCCTGTGGCTGCTGAGCTGCTGGGCCCTGCTGGGGACCAC |
| | ATT TGGC (SEQ ID NO: 5) |
| AHSG | MKSLVLLLCLAQLWGCHS (SEQ ID NO: 57) |
| Signal | |
| AHSG | ATGAAGTCCCTCGTCCTGCTCCTTTGTCTTGCTCAGCTCTGGGGCTGC |
| Signal | CACTCA (SEQ ID NO: 6) |
| CD300 | MWLPWALLLLWVPGCFA (SEQ ID NO: 58) |
| Signal | |
| CD300 | ATGTGGCTGCCTTGGGCTCTGTTGCTTCTCTGGGTCCCAGGATGTTTT |
| Signal | GCT (SEQ ID NO: 7) |
| GLA | MQLRNPELHLGCALALRFLALVSWDIPGARA (SEQ ID NO: 59) |
| Signal | |
| GLA | ATGCAGCTGAGGAACCCAGAACTACATCTGGGCTGCGCGCTTGCGCT |
| Signal | TCGCTTCCTGGCCCTCGTTTCCTGGGACATCCCTGGGGCTAGAGCA |
| | (SEQ ID NO: 8) |
| LAMP1 | MAAPGSARRPLLLLLLLLLLGLMHCASA (SEQ ID NO: 60) |
| LAMP1 | ATGGCGGCCCCCGGCAGCGCCCGGCGACCCCTGCTGCTGCTACTGCT |
| | GTTGCTGCTGCTCGGCCTCATGCATTGTGCGTCAGCA (SEQ ID NO: 9) |
| Notch2 | MPALRPALLWALLALWLCCAAPAHA (SEQ ID NO: 61) |
| Signal | |
| Notch2 | ATGCCCGCCCTGCGCCCCGCTCTGCTGTGGGCGCTGCTGGCGCTCTGG |
| Signal | CTGTGCTGCGCGGCCCCCGCGCATGCA (SEQ ID NO: 10) |
| ORM1 | MALSWVLTVLSLLPLLEA (SEQ ID NO: 62) |
| Signal | |
| ORM1 | ATGGCGCTGTCCTGGGTTCTTACAGTCCTGAGCCTCCTACCTCTGCTG |
| Signal | GAAGCC (SEQ ID NO: 11) |
| TF | MRLAVGALLVCAVLGLCLA (SEQ ID NO: 63) |
| Signal | |
| TF | ATGAGGCTCGCCGTGGGAGCCCTGCTGGTCTGCGCCGTCCTGGGGCT |
| Signal | GTGTCTGGCT (SEQ ID NO: 13) |
| GLAco4 | CTGGACAATGGCCTGGCCAGGACCCCCACCATGGGGTGGCTGCACTG |
| | GGAGAGGTTCATGTGCAACCTGGATTGCCAGGAGGAGCCAGATTCTT |
| | GCATCTCTGAGAAGCTGTTCATGGAGATGGCTGAGCTGATGGTGTCT |
| | GAGGGCTGGAAGGATGCTGGCTATGAGTACCTGTGCATTGATGACTG |
| | CTGGATGGCTCCCCAGAGGGACTCTGAGGGCAGACTGCAGGCTGACC |
| | CCCAGAGGTTCCCCCATGGGATCAGGCAGCTGGCCAACTATGTGCAC |
| | AGCAAGGGCCTGAAGCTGGGCATTTATGCTGATGTGGGCAACAAGAC |
| | CTGTGCTGGCTTCCCTGGCTCTTTTGGCTATTATGACATTGATGCTCA |
| | GACCTTTGCTGACTGGGGGGTGGACCTGCTGAAGTTTGATGGGTGCT |
| | ATTGTGACTCTCTGGAGAACCTGGCTGATGGCTATAAGCATATGTCTC |
| | TGGCCCTGAACAGGACTGGCAGGAGCATTGTCTACAGCTGTGAGTGG |
| | CCTCTGTACATGTGGCCTTTCCAGAAGCCTAACTACACTGAGATCAG |
| | GCAGTATTGCAACCACTGGAGGAACTTTGCTGACATTGATGACTCTT |
| | GGAAGTCTATTAAGAGCATTCTGGATTGGACCAGCTTCAACCAGGAA |
| | AGGATTGTGGATGTGGCTGGGCCTGGGGGCTGGAATGACCCTGACAT |
| | GCTGGTGATTGGGAACTTTGGCCTGAGCTGGAACCAGCAGGTGACCC |
| | AGATGGCCCTGTGGGCCATCATGGCTGCCCCCCTGTTCATGTCCAATG |
| | ACCTGAGGCACATCAGCCCCCAGGCCAAGGCCCTGCTGCAGGATAAG |
| | GATGTGATTGCCATCAATCAGGACCCCCTGGGGAAGCAGGGCTACCA |
| | GCTGAGGCAGGGGGACAACTTTGAGGTGTGGGAGAGACCTCTGTCTG |
| | GGCTGGCCTGGGCTGTGGCCATGATCAACAGGCAGGAGATTGGGGG |
| | CCCCAGGAGCTATACCATTGCTGTGGCCTCTCTGGGGAAGGGGGTGG |
| | CCTGCAACCCTGCCTGCTTCATCACCCAGCTGCTGCCTGTGAAGAGG |
| | AAGCTGGGCTTCTATGAGTGGACCAGCAGGCTGAGGAGCCACATTAA |
| | TCCCACTGGCACTGTGCTGCTGCAGCTGGAGAACACCATGCAGATGT |
| | CTCTGAAGGATCTGCTGTGATAA (SEQ ID NO: 15) |
| GLAcoBC0 | CTGGACAATGGCCTGGCCAGAACCCCCACCATGGGCTGGCTGCACTG |
| | GGAGAGGTTCATGTGCAACCTTGACTGCCAGGAGGAGCCTGACAGCT |
| | GCATCTCTGAGAAGCTGTTCATGGAGATGGCTGAGCTCATGGTGTCA |
| | GAGGGGTGGAAGGATGCAGGCTATGAGTACCTGTGCATAGATGACT |
| | GCTGGATGGCTCCCCAGAGGGACTCAGAGGGCAGGCTGCAGGCTGA |
| | CCCTCAGAGGTTCCCCCATGGCATCAGGCAGCTGGCCAACTATGTGC |
| | ACAGCAAGGGCCTCAAGCTGGGAATCTATGCTGATGTGGGCAACAA |
| | AACATGTGCAGGCTTCCCAGGCTCTTTTGGCTACTATGACATAGATGC |
| | CCAGACCTTTGCAGACTGGGGAGTTGACCTGCTGAAGTTTGATGGGT |
| | GCTACTGTGACAGCCTGGAGAACCTGGCTGATGGGTACAAGCACATG |
| | TCCCTGGCCCTGAACAGGACAGGCAGGTCCATTGTGTACAGCTGTGA |
| | GTGGCCCCTGTACATGTGGCCCTTCCAGAAGCCCAACTACACAGAGA |
| | TCAGACAGTACTGCAACCACTGGAGAAACTTTGCTGACATTGATGAC |
| | TCCTGGAAGAGCATCAAGTCCATCCTGGACTGGACCAGCTTCAACCA |
| | GGAGAGGATTGTGGATGTGGCTGGCCCAGGGGGCTGGAATGACCCT |
| | GACATGCTGGTCATTGGCAACTTTGGCCTGAGCTGGAACCAGCAGGT |
| | GACCCAGATGGCCCTGTGGGCCATCATGGCTGCCCCCCTCTTCATGA |
| | GCAATGACCTGAGACACATCTCCCCCCAAGCCAAGGCCCTGTTGCAG |
| | GACAAGGATGTGATTGCCATCAATCAGGACCCCTTGGGCAAGCAGGG |
| | CTACCAGCTGAGACAGGGGGACAACTTTGAGGTGTGGGAGAGACCC |
| | CTCTCTGGCTTGGCCTGGGCTGTGGCCATGATCAACAGACAGGAGAT |
| | TGGGGGCCCCAGAAGCTACACCATTGCTGTTGCCAGCCTGGGCAAGG |
| | GGGTGGCCTGCAACCCTGCCTGCTTCATCACACAGCTGCTGCCTGTG |
| | AAGAGGAAGCTGGGGTTCTATGAGTGGACCTCTAGGCTGAGAAGTCA |
| | CATCAACCCCACAGGCACAGTGCTGCTGCAGTTGGAGAACACCATGC |
| | AGATGAGCCTGAAGGACCTGCTCTGA (SEQ ID NO: 16) |
| GLAcoH0 | CTGGACAATGGTCTGGCCAGGACTCCCACCATGGGCTGGCTGCACTG |
| | GGAGAGGTTCATGTGCAACCTGGACTGCCAGGAGGAGCCAGACTCCT |
| | GCATCAGTGAGAAGCTCTTCATGGAGATGGCTGAGCTCATGGTCTCT |
| | GAAGGGTGGAAGGATGCTGGGTATGAGTACCTGTGTATTGATGACTG |
| | CTGGATGGCCCCCCAAAGGGACTCAGAGGGCAGGCTGCAGGCAGAC |
| | CCTCAGAGGTTCCCTCATGGGATCAGGCAGCTTGCCAACTATGTCCA |
| | CAGCAAGGGGCTGAAGCTGGGGATCTATGCTGATGTTGGGAACAAG |
| | ACCTGTGCTGGCTTCCCAGGCTCCTTTGGCTACTATGATATTGATGCA |
| | CAGACATTTGCAGATTGGGGGGTGGATCTGCTGAAGTTTGATGGGTG |
| | CTACTGTGACTCCCTGGAGAACCTTGCAGATGGGTACAAGCACATGT |
| | CCCTGGCCCTGAACAGGACTGGCAGAAGCATAGTCTACTCCTGTGAG |
| | TGGCCCCTGTACATGTGGCCCTTCCAGAAGCCCAACTACACAGAGAT |
| | CAGACAGTACTGCAACCACTGGAGGAACTTTGCAGACATTGATGATT |
| | CCTGGAAGAGTATCAAGAGCATCCTAGACTGGACAAGCTTTAACCAG |
| | GAGAGGATTGTGGATGTAGCAGGCCCAGGGGGCTGGAATGACCCAG |
| | ACATGCTTGTGATTGGCAACTTTGGCCTCAGCTGGAACCAGCAGGTG |
| | ACTCAGATGGCCCTGTGGGCCATCATGGCTGCCCCCCTGTTCATGTCT |
| | AATGACCTCAGGCACATCAGCCCACAAGCCAAGGCACTCCTCCAGGA |
| | CAAGGATGTGATAGCCATCAACCAGGACCCCTTGGGGAAGCAGGGG |
| | TACCAGCTAAGGCAGGGGGACAACTTTGAGGTGTGGGAAAGGCCCC |
| | TCTCAGGGCTTGCCTGGGCAGTAGCTATGATCAACAGGCAGGAGATT |
| | GGTGGCCCCAGATCTTACACTATTGCAGTGGCTAGCCTGGGGAAGGG |
| | GGTGGCCTGCAACCCTGCCTGCTTCATCACACAGCTCCTCCCAGTCAA |
| | GAGGAAGTTGGGGTTCTATGAGTGGACTTCCAGGCTGAGATCCCACA |
| | TCAACCCCACAGGCACTGTGCTGCTGCAGCTAGAGAACACCATGCAG |
| | ATGAGCCTGAAGGACCTCCTCTGA (SEQ ID NO: 17) |
| GLAcoH6 | CTGGACAATGGCTTGGCCAGGACTCCCACCATGGGCTGGCTGCACTG |
| | GGAGAGGTTCATGTGCAACTTGGACTGCCAGGAGGAGCCAGACTCCT |
| | GCATCTCTGAGAAGCTCTTCATGGAGATGGCTGAGCTCATGGTCTCA |
| | GAGGGGTGGAAGGATGCAGGGTATGAGTACCTCTGCATTGATGACTG |
| | CTGGATGGCCCCTCAAAGGGATTCAGAGGGTAGGCTCCAGGCAGACC |
| | CCCAGAGGTTCCCCCATGGCATCAGGCAGCTTGCTAACTATGTCCAC |
| | AGCAAGGGCCTGAAGCTGGGGATCTATGCTGATGTTGGCAACAAGAC |
| | CTGTGCTGGCTTCCCAGGTTCCTTTGGGTACTATGACATTGATGCACA |
| | GACCTTTGCAGACTGGGGGGTAGACCTCCTAAAGTTTGATGGGTGCT |
| | ACTGTGACAGCTTGGAGAACCTGGCAGATGGCTACAAGCACATGAGC |
| | CTTGCCCTGAACAGGACTGGCAGAAGCATAGTGTACTCCTGTGAGTG |
| | GCCCCTGTACATGTGGCCCTTCCAGAAGCCCAACTACACAGAGATCA |
| | GACAGTACTGCAACCACTGGAGGAACTTTGCTGACATAGATGACTCC |
| | TGGAAGAGCATAAAGAGCATCCTGGACTGGACATCCTTCAACCAGGA |
| | GAGGATTGTGGATGTGGCTGGGCCAGGGGGCTGGAATGACCCAGAC |
| | ATGCTTGTGATAGGCAACTTTGGCCTCAGCTGGAACCAGCAGGTGAC |
| | TCAGATGGCCCTCTGGGCCATCATGGCTGCCCCCCTGTTCATGAGCA |
| | ATGACCTCAGGCACATCAGCCCCCAAGCCAAGGCCCTCCTCCAGGAC |
| | AAGGATGTGATAGCCATCAATCAGGACCCCCTAGGCAAGCAGGGGT |
| | ACCAGCTCAGACAGGGTGACAACTTTGAGGTGTGGGAAAGGCCTCTG |
| | TCAGGCCTTGCCTGGGCAGTGGCTATGATCAACAGGCAGGAGATTGG |
| | TGGGCCTAGGAGCTACACCATTGCAGTGGCCTCCCTGGGGAAGGGAG |
| | TGGCCTGCAACCCAGCCTGCTTCATCACACAGCTCCTCCCTGTGAAG |
| | AGGAAGCTTGGCTTCTATGAATGGACCTCCAGGCTGAGGAGTCACAT |
| | CAACCCCACAGGGACTGTCCTCCTACAGCTAGAGAACACCATGCAGA |
| | TGTCCCTGAAGGACCTGCTGTGA (SEQ ID NO: 18) |
| GLAvar45v2 | CTGGACAATGGCCTGGCCAGGACCCCCACCATGGGCTGGCTGCACTG |
| | GGAGAGATTCATGTGCAACCTGGACTGCCAGGAGGAGCCTGACAGCT |
| | GCATCTCTGAGAAGCTGTTCATGGAGATGGCAGAGCTGATGGTGTCT |
| | GAGGGCTGGAAGGATGCTGGCTATGAGTACCTGTGCATTGATGACTG |
| | CTGGATGGCCCCCCAGAGAGACTCTGAGGGCAGGCTGCAGGCAGAC |
| | CCCCAGAGGTTCCCCCATGGCATCAGACAGCTGGCCAACTATGTGCA |
| | CAGCAAGGGCCTGAAGCTGGGCATCTATGCTGATGTGGGCAACAAG |
| | ACCTGCGCAGGCTTCCCTGGCTCCTTTGGCTACTATGACATTGATGCC |
| | CAGACCTTTGCTGACTGGGGAGTGGACCTGCTGAAGTTTGATGGCTG |
| | CTACTGTGACTCCCTGGAGAACCTGGCTGATGGCTACAAGCACATGT |
| | CCCTGGCCCTGAACAGGACAGGCAGATCCATTGTGTACAGCTGTGAG |
| | TGGCCCCTGTACATGTGGCCATTCCAGAAGCCCAACTACACAGAGAT |
| | CAGGCAGTACTGCAACCACTGGAGAAACTTTGCAGACATTGATGATT |
| | CCTGGAAGAGCATCAAGTCCATCCTGGACTGGACCTCCTTCAACCAG |
| | GAGAGAATTGTGGATGTGGCAGGCCCTGGGGGCTGGAATGACCCAG |
| | ACATGCTGGTCATTGGCAACTTTGGCCTGTCCTGGAACCAGCAGGTG |
| | ACCCAGATGGCCCTGTGGGCCATCATGGCAGCCCCCCTGTTCATGAG |
| | CAATGACCTGAGACACATCAGCCCACAGGCCAAGGCCCTGCTGCAGG |
| | ACAAGGATGTGATTGCCATCAACCAGGACCCTCTGGGCAAGCAGGGC |
| | TACCAGCTGAGGCAGGGAGACAACTTTGAGGTGTGGGAGAGGCCCC |
| | TGTCTGGCCTGGCCTGGGCAGTGGCCATGATCAACAGGCAGGAGATT |
| | GGAGGCCCCAGGAGCTACACCATTGCAGTGGCCAGCCTGGGCAAGG |
| | GGGTGGCCTGCAACCCAGCCTGCTTCATCACCCAGCTGCTGCCTGTG |
| | AAGAGGAAGCTGGGCTTCTATGAGTGGACCTCCAGACTGAGGAGCC |
| | ACATCAACCCCACAGGCACAGTGCTGCTGCAGCTGGAGAACACCATG |
| | CAGATGAGCCTGAAGGACCTGCTGTGA (SEQ ID NO: 19) |
| VP1 | MAADGYLPDWLEDNLSEGIREWWDLKPGAPKPKANQQKQDNGRGLVL |
| | PGYKYLGPFNGLDKGEPVNAADAAALEHDKAYDQQLQAGDNPYLRYN |
| | HADAEFQERLQEDTSFGGNLGRAVFQAKKRVLEPLGLVESPVKTAPGK |
| | KRPVEPSPQRSPDSSTGIGKKGQQPAKKRLNFGQTGDSESVPDPQPIGEPP |
| | AAPSGVGPNTMAAGGGAPMADNNEGADGVGSSSGNWHCDSTWLGDR |
| | VITTSTRTWALPTYNNHLYKQISNGTSGGSTNDNTYFGYSTPWGYFDFN |
| | RFHCHFSPRDWQRLINNNWGFRPKRLNFKLFNIQVKEVTQNEGTKTIAN |
| | NLTSTIQVFTDSEYQLPYVLGSAHQGCLPPFPADVFMIPQYGYLTLNNGS |
| | QAVGRSSFYCLEYFPSQMLRTGNNFEFSYNFEDVPFHSSYAHSQSLDRL |
| | MNPLIDQYLYYLSRTQSTGGTAGTQQLLFSQAGPNNMSAQAKNWLPGP |
| | CYRQQRVSTTLSQNNNSNFAWTGATKYHLNGRDSLVNPGVAMATHKD |
| | DEERFFPSSGVLMFGKQGAGKDNVDYSSVMLTSEEEIKTTNPVATEQYG |
| | VVADNLQQQNAAPIVGAVNSQGALPGMVWQNRDVYLQGPIWAKIPHT |
| | DGNFHPSPLMGGFGLKHPPPQILIKNTPVPADPPTTFNQAKLASFITQYST |
| | GQVSVEIEWELQKENSKRWNPEIQYTSNYYKSTNVDFAVNTEGTYSEPR |
| | PIGTRYLTRNL (SEQ ID NO: 110) |
| VP1 | MAADGYLPDWLEDNLSEGIREWWALQPGAPKPKANQQHQDNARGLVL |
| | PGYKYLGPGNGLDKGEPVNAADAAALEHDKAYDQQLKAGDNPYLKY |
| | NHADAEFQERLKEDTSFGGNLGRAVFQAKKRLLEPLGLVEEAAKTAPG |
| | KKRPVDQSPQEPDSSSGVGKSGKQPARKRLNFGQTGDSESVPDPQPLGE |
| | PPAAPTSLGSNTMASGGGAPMADNNEGADGVGNSSGNWHCDSQWLGD |
| | RVITTSTRTWALPTYNNHLYKQISSQSGASNDNHYFGYSTPWGYFDFNR |
| | FHCHFSPRDWQRLINNNWGFRPKKLSFKLFNIQVKEVTQNDGTTTIANN |
| | LTSTVQVFTDSEYQLPYVLGSAHQGCLPPFPADVFMVPQYGYLTLNNGS |
| | QAVGRSSFYCLEYFPSQMLRTGNNFQFSYTFEDVPFHSSYAHSQSLDRL |
| | MNPLIDQYLYYLNRTQGTTSGTTNQSRLLFSQAGPQSMSLQARNWLPGP |
| | CYRQQRLSKTANDNNNSNFPWTAASKYHLNGRDSLVNPGPAMASHKD |
| | DEEKFFPMHGNLIFGKEGTTASNAELDNVMITDEEEIRTTNPVATEQYGT |
| | VANNLQSSNTAPTTRTVNDQGALPGMVWQDRDVYLQGPIWAKIPHTD |
| | GHFHPSPLMGGFGLKHPPPQIMIKNTPVPANPPTTFSPAKFASFITQYSTG |
| | QVSVEIEWELQKENSKRWNPEIQYTSNYNKSVNVDFTVDINGVYSEPRP |
| | IGTRYLTRPL (SEQ ID NO: 42) |
| pAAV_ApoE_ | CCTGCAGGCAGCTGCGCGCTCGCTCGCTCACTGAGGCCGCCCGGGCA |
| hAAT_ | AAGCCCGGGCGGCCTCAGTGAGCGAGCGAGCGCGCAGAGAGGGAGT |
| HBB2m1_sp7. | GGCCAACTCCATCACTAGGGGTTCCTGCGGCCTAGTAGGCTCAGAGG |
| GLAco4_ | CACACAGGAGTTTCTGGGCTCACCCTGCCCCCTTCCAACCCCTCAGTT |
| BGH228 | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGCTCCTGGGCAACCTGCTGGTCTCTC |
| | TGCTGGCCCATCACTTTGGCAAAGCACGCGTGCCACCATGGCCTTTCT |
| | GTGGCTGCTGTCCTGCTGGGCCCTGCTGGGGACCACCTTTGGCCTGG |
| | ACAATGGCCTGGCCAGGACCCCCACCATGGGGTGGCTGCACTGGGAG |
| | AGGTTCATGTGCAACCTGGATTGCCAGGAGGAGCCAGATTCTTGCAT |
| | CTCTGAGAAGCTGTTCATGGAGATGGCTGAGCTGATGGTGTCTGAGG |
| | GCTGGAAGGATGCTGGCTATGAGTACCTGTGCATTGATGACTGCTGG |
| | ATGGCTCCCCAGAGGGACTCTGAGGGCAGACTGCAGGCTGACCCCCA |
| | GAGGTTCCCCCATGGGATCAGGCAGCTGGCCAACTATGTGCACAGCA |
| | AGGGCCTGAAGCTGGGCATTTATGCTGATGTGGGCAACAAGACCTGT |
| | GCTGGCTTCCCTGGCTCTTTTGGCTATTATGACATTGATGCTCAGACC |
| | TTTGCTGACTGGGGGGTGGACCTGCTGAAGTTTGATGGGTGCTATTGT |
| | GACTCTCTGGAGAACCTGGCTGATGGCTATAAGCATATGTCTCTGGC |
| | CCTGAACAGGACTGGCAGGAGCATTGTCTACAGCTGTGAGTGGCCTC |
| | TGTACATGTGGCCTTTCCAGAAGCCTAACTACACTGAGATCAGGCAG |
| | TATTGCAACCACTGGAGGAACTTTGCTGACATTGATGACTCTTGGAA |
| | GTCTATTAAGAGCATTCTGGATTGGACCAGCTTCAACCAGGAAAGGA |
| | TTGTGGATGTGGCTGGGCCTGGGGGCTGGAATGACCCTGACATGCTG |
| | GTGATTGGGAACTTTGGCCTGAGCTGGAACCAGCAGGTGACCCAGAT |
| | GGCCCTGTGGGCCATCATGGCTGCCCCCCTGTTCATGTCCAATGACCT |
| | GAGGCACATCAGCCCCCAGGCCAAGGCCCTGCTGCAGGATAAGGAT |
| | GTGATTGCCATCAATCAGGACCCCCTGGGGAAGCAGGGCTACCAGCT |
| | GAGGCAGGGGGACAACTTTGAGGTGTGGGAGAGACCTCTGTCTGGG |
| | CTGGCCTGGGCTGTGGCCATGATCAACAGGCAGGAGATTGGGGGCCC |
| | CAGGAGCTATACCATTGCTGTGGCCTCTCTGGGGAAGGGGGTGGCCT |
| | GCAACCCTGCCTGCTTCATCACCCAGCTGCTGCCTGTGAAGAGGAAG |
| | CTGGGCTTCTATGAGTGGACCAGCAGGCTGAGGAGCCACATTAATCC |
| | CACTGGCACTGTGCTGCTGCAGCTGGAGAACACCATGCAGATGTCTC |
| | TGAAGGATCTGCTGTGATAAAGATCTAGAGCTGAATTCCTGCAGCCA |
| | GGGGGATCAGCCTCTACTGTGCCTTCTAGTTGCCAGCCATCTGTTGTT |
| | TGCCCCTCCCCCTTGCCTTCCTTGACCCTGGAAGGTGCCACTCCCACT |
| | GTCCTTTCCTAATAAAATGAGGAAATTGCATCACATTGTCTGAGTAG |
| | GTGTCATTCTATTCTGGGGGGTGGGGTGGGGCAGGACAGCAAGGGG |
| | GAGGATTGGGAAGACAATAGCAGGCATGCTGGGGATGCAGTGGGCT |
| | CTATGGCTTCTGAGGCAGAAAGAACCAGCTGGGGCTCGAGATCCACT |
| | AGGGCCGCAGGAACCCCTAGTGATGGAGTTGGCCACTCCCTCTCTGC |
| | GCGCTCGCTCGCTCACTGAGGCCGGGCGACCAAAGGTCGCCCGACGC |
| | CCGGGCTTTGCCCGGGCGGCCTCAGTGAGCGAGCGAGCGCGCAGCTG |
| | CCTGCAGG (SEQ. ID. NO: 91) |
| pAAV_ApoE_ | CCTGCAGGCAGCTGCGCGCTCGCTCGCTCACTGAGGCCGCCCGGGCA |
| hAAT_ | AAGCCCGGGCGGCCTCAGTGAGCGAGCGAGCGCGCAGAGAGGGAGT |
| HBB2m1_sp7. | GGCCAACTCCATCACTAGGGGTTCCTGCGGCCTAGTAGGCTCAGAGG |
| GLAcoBC0_ | CACACAGGAGTTTCTGGGCTCACCCTGCCCCCTTCCAACCCCTCAGTT |
| BGH228 | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGCTCCTGGGCAACCTGCTGGTCTCTC |
| | TGCTGGCCCATCACTTTGGCAAAGCACGCGTGCCACCATGGCCTTCCT |
| | GTGGCTGCTGAGCTGCTGGGCCCTGCTGGGGACCACCTTTGGGCTGG |
| | ACAATGGCCTGGCCAGAACCCCCACCATGGGCTGGCTGCACTGGGAG |
| | AGGTTCATGTGCAACCTTGACTGCCAGGAGGAGCCTGACAGCTGCAT |
| | CTCTGAGAAGCTGTTCATGGAGATGGCTGAGCTCATGGTGTCAGAGG |
| | GGTGGAAGGATGCAGGCTATGAGTACCTGTGCATAGATGACTGCTGG |
| | ATGGCTCCCCAGAGGGACTCAGAGGGCAGGCTGCAGGCTGACCCTCA |
| | GAGGTTCCCCCATGGCATCAGGCAGCTGGCCAACTATGTGCACAGCA |
| | AGGGCCTCAAGCTGGGAATCTATGCTGATGTGGGCAACAAAACATGT |
| | GCAGGCTTCCCAGGCTCTTTTGGCTACTATGACATAGATGCCCAGAC |
| | CTTTGCAGACTGGGGAGTTGACCTGCTGAAGTTTGATGGGTGCTACT |
| | GTGACAGCCTGGAGAACCTGGCTGATGGGTACAAGCACATGTCCCTG |
| | GCCCTGAACAGGACAGGCAGGTCCATTGTGTACAGCTGTGAGTGGCC |
| | CCTGTACATGTGGCCCTTCCAGAAGCCCAACTACACAGAGATCAGAC |
| | AGTACTGCAACCACTGGAGAAACTTTGCTGACATTGATGACTCCTGG |
| | AAGAGCATCAAGTCCATCCTGGACTGGACCAGCTTCAACCAGGAGAG |
| | GATTGTGGATGTGGCTGGCCCAGGGGGCTGGAATGACCCTGACATGC |
| | TGGTCATTGGCAACTTTGGCCTGAGCTGGAACCAGCAGGTGACCCAG |
| | ATGGCCCTGTGGGCCATCATGGCTGCCCCCCTCTTCATGAGCAATGA |
| | CCTGAGACACATCTCCCCCCAAGCCAAGGCCCTGTTGCAGGACAAGG |
| | ATGTGATTGCCATCAATCAGGACCCCTTGGGCAAGCAGGGCTACCAG |
| | CTGAGACAGGGGGACAACTTTGAGGTGTGGGAGAGACCCCTCTCTGG |
| | CTTGGCCTGGGCTGTGGCCATGATCAACAGACAGGAGATTGGGGGCC |
| | CCAGAAGCTACACCATTGCTGTTGCCAGCCTGGGCAAGGGGGTGGCC |
| | TGCAACCCTGCCTGCTTCATCACACAGCTGCTGCCTGTGAAGAGGAA |
| | GCTGGGGTTCTATGAGTGGACCTCTAGGCTGAGAAGTCACATCAACC |
| | CCACAGGCACAGTGCTGCTGCAGTTGGAGAACACCATGCAGATGAGC |
| | CTGAAGGACCTGCTCTGAAGATCTAGAGCTGAATTCCTGCAGCCAGG |
| | GGGATCAGCCTCTACTGTGCCTTCTAGTTGCCAGCCATCTGTTGTTTG |
| | CCCCTCCCCCTTGCCTTCCTTGACCCTGGAAGGTGCCACTCCCACTGT |
| | CCTTTCCTAATAAAATGAGGAAATTGCATCACATTGTCTGAGTAGGT |
| | GTCATTCTATTCTGGGGGGTGGGGTGGGGCAGGACAGCAAGGGGGA |
| | GGATTGGGAAGACAATAGCAGGCATGCTGGGGATGCAGTGGGCTCT |
| | ATGGCTTCTGAGGCAGAAAGAACCAGCTGGGGCTCGAGATCCACTAG |
| | GGCCGCAGGAACCCCTAGTGATGGAGTTGGCCACTCCCTCTCTGCGC |
| | GCTCGCTCGCTCACTGAGGCCGGGCGACCAAAGGTCGCCCGACGCCC |
| | GGGCTTTGCCCGGGCGGCCTCAGTGAGCGAGCGAGCGCGCAGCTGCC |
| | TGCAGG (SEQ. ID. NO: 92) |
| pAAV_ApoE_ | CCTGCAGGCAGCTGCGCGCTCGCTCGCTCACTGAGGCCGCCCGGGCA |
| hAAT_ | AAGCCCGGGCGGCCTCAGTGAGCGAGCGAGCGCGCAGAGAGGGAGT |
| HBB2m1_sp7. | GGCCAACTCCATCACTAGGGGTTCCTGCGGCCTAGTAGGCTCAGAGG |
| GLAcoH0_ | CACACAGGAGTTTCTGGGCTCACCCTGCCCCCTTCCAACCCCTCAGTT |
| BGH228 | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGCTCCTGGGCAACCTGCTGGTCTCTC |
| | TGCTGGCCCATCACTTTGGCAAAGCACGCGTGCCACCATGGCCTTCCT |
| | GTGGCTCCTGAGCTGCTGGGCCCTCCTGGGGACCACCTTTGGGCTGG |
| | ACAATGGTCTGGCCAGGACTCCCACCATGGGCTGGCTGCACTGGGAG |
| | AGGTTCATGTGCAACCTGGACTGCCAGGAGGAGCCAGACTCCTGCAT |
| | CAGTGAGAAGCTCTTCATGGAGATGGCTGAGCTCATGGTCTCTGAAG |
| | GGTGGAAGGATGCTGGGTATGAGTACCTGTGTATTGATGACTGCTGG |
| | ATGGCCCCCCAAAGGGACTCAGAGGGCAGGCTGCAGGCAGACCCTC |
| | AGAGGTTCCCTCATGGGATCAGGCAGCTTGCCAACTATGTCCACAGC |
| | AAGGGGCTGAAGCTGGGGATCTATGCTGATGTTGGGAACAAGACCTG |
| | TGCTGGCTTCCCAGGCTCCTTTGGCTACTATGATATTGATGCACAGAC |
| | ATTTGCAGATTGGGGGGTGGATCTGCTGAAGTTTGATGGGTGCTACT |
| | GTGACTCCCTGGAGAACCTTGCAGATGGGTACAAGCACATGTCCCTG |
| | GCCCTGAACAGGACTGGCAGAAGCATAGTCTACTCCTGTGAGTGGCC |
| | CCTGTACATGTGGCCCTTCCAGAAGCCCAACTACACAGAGATCAGAC |
| | AGTACTGCAACCACTGGAGGAACTTTGCAGACATTGATGATTCCTGG |
| | AAGAGTATCAAGAGCATCCTAGACTGGACAAGCTTTAACCAGGAGA |
| | GGATTGTGGATGTAGCAGGCCCAGGGGGCTGGAATGACCCAGACAT |
| | GCTTGTGATTGGCAACTTTGGCCTCAGCTGGAACCAGCAGGTGACTC |
| | AGATGGCCCTGTGGGCCATCATGGCTGCCCCCCTGTTCATGTCTAATG |
| | ACCTCAGGCACATCAGCCCACAAGCCAAGGCACTCCTCCAGGACAAG |
| | GATGTGATAGCCATCAACCAGGACCCCTTGGGGAAGCAGGGGTACC |
| | AGCTAAGGCAGGGGGACAACTTTGAGGTGTGGGAAAGGCCCCTCTC |
| | AGGGCTTGCCTGGGCAGTAGCTATGATCAACAGGCAGGAGATTGGTG |
| | GCCCCAGATCTTACACTATTGCAGTGGCTAGCCTGGGGAAGGGGGTG |
| | GCCTGCAACCCTGCCTGCTTCATCACACAGCTCCTCCCAGTCAAGAG |
| | GAAGTTGGGGTTCTATGAGTGGACTTCCAGGCTGAGATCCCACATCA |
| | ACCCCACAGGCACTGTGCTGCTGCAGCTAGAGAACACCATGCAGATG |
| | AGCCTGAAGGACCTCCTCTGAAGATCTAGAGCTGAATTCCTGCAGCC |
| | AGGGGGATCAGCCTCTACTGTGCCTTCTAGTTGCCAGCCATCTGTTGT |
| | TTGCCCCTCCCCCTTGCCTTCCTTGACCCTGGAAGGTGCCACTCCCAC |
| | TGTCCTTTCCTAATAAAATGAGGAAATTGCATCACATTGTCTGAGTAG |
| | GTGTCATTCTATTCTGGGGGGTGGGGTGGGGCAGGACAGCAAGGGG |
| | GAGGATTGGGAAGACAATAGCAGGCATGCTGGGGATGCAGTGGGCT |
| | CTATGGCTTCTGAGGCAGAAAGAACCAGCTGGGGCTCGAGATCCACT |
| | AGGGCCGCAGGAACCCCTAGTGATGGAGTTGGCCACTCCCTCTCTGC |
| | GCGCTCGCTCGCTCACTGAGGCCGGGCGACCAAAGGTCGCCCGACGC |
| | CCGGGCTTTGCCCGGGCGGCCTCAGTGAGCGAGCGAGCGCGCAGCTG |
| | CCTGCAGG (SEQ. ID. NO: 93) |
| pAAV_ApoE_ | CCTGCAGGCAGCTGCGCGCTCGCTCGCTCACTGAGGCCGCCCGGGCA |
| hAAT_ | AAGCCCGGGCGGCCTCAGTGAGCGAGCGAGCGCGCAGAGAGGGAGT |
| HBB2m1_sp7. | GGCCAACTCCATCACTAGGGGTTCCTGCGGCCTAGTAGGCTCAGAGG |
| GLAcoH6_ | CACACAGGAGTTTCTGGGCTCACCCTGCCCCCTTCCAACCCCTCAGTT |
| BGH228 | CCCATCCTCCAGCAGCTGTTTGTGTGCTGCCTCTGAAGTCCACACTGA |
| sequence | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGCTCCTGGGCAACCTGCTGGTCTCTC |
| | TGCTGGCCCATCACTTTGGCAAAGCACGCGTGCCACCATGGCCTTCCT |
| | GTGGCTGCTGAGCTGCTGGGCCCTGCTGGGGACCACATTTGGCCTGG |
| | ACAATGGCTTGGCCAGGACTCCCACCATGGGCTGGCTGCACTGGGAG |
| | AGGTTCATGTGCAACTTGGACTGCCAGGAGGAGCCAGACTCCTGCAT |
| | CTCTGAGAAGCTCTTCATGGAGATGGCTGAGCTCATGGTCTCAGAGG |
| | GGTGGAAGGATGCAGGGTATGAGTACCTCTGCATTGATGACTGCTGG |
| | ATGGCCCCTCAAAGGGATTCAGAGGGTAGGCTCCAGGCAGACCCCCA |
| | GAGGTTCCCCCATGGCATCAGGCAGCTTGCTAACTATGTCCACAGCA |
| | AGGGCCTGAAGCTGGGGATCTATGCTGATGTTGGCAACAAGACCTGT |
| | GCTGGCTTCCCAGGTTCCTTTGGGTACTATGACATTGATGCACAGACC |
| | TTTGCAGACTGGGGGGTAGACCTCCTAAAGTTTGATGGGTGCTACTG |
| | TGACAGCTTGGAGAACCTGGCAGATGGCTACAAGCACATGAGCCTTG |
| | CCCTGAACAGGACTGGCAGAAGCATAGTGTACTCCTGTGAGTGGCCC |
| | CTGTACATGTGGCCCTTCCAGAAGCCCAACTACACAGAGATCAGACA |
| | GTACTGCAACCACTGGAGGAACTTTGCTGACATAGATGACTCCTGGA |
| | AGAGCATAAAGAGCATCCTGGACTGGACATCCTTCAACCAGGAGAG |
| | GATTGTGGATGTGGCTGGGCCAGGGGGCTGGAATGACCCAGACATGC |
| | TTGTGATAGGCAACTTTGGCCTCAGCTGGAACCAGCAGGTGACTCAG |
| | ATGGCCCTCTGGGCCATCATGGCTGCCCCCCTGTTCATGAGCAATGA |
| | CCTCAGGCACATCAGCCCCCAAGCCAAGGCCCTCCTCCAGGACAAGG |
| | ATGTGATAGCCATCAATCAGGACCCCCTAGGCAAGCAGGGGTACCAG |
| | CTCAGACAGGGTGACAACTTTGAGGTGTGGGAAAGGCCTCTGTCAGG |
| | CCTTGCCTGGGCAGTGGCTATGATCAACAGGCAGGAGATTGGTGGGC |
| | CTAGGAGCTACACCATTGCAGTGGCCTCCCTGGGGAAGGGAGTGGCC |
| | TGCAACCCAGCCTGCTTCATCACACAGCTCCTCCCTGTGAAGAGGAA |
| | GCTTGGCTTCTATGAATGGACCTCCAGGCTGAGGAGTCACATCAACC |
| | CCACAGGGACTGTCCTCCTACAGCTAGAGAACACCATGCAGATGTCC |
| | CTGAAGGACCTGCTGTGAAGATCTAGAGCTGAATTCCTGCAGCCAGG |
| | GGGATCAGCCTCTACTGTGCCTTCTAGTTGCCAGCCATCTGTTGTTTG |
| | CCCCTCCCCCTTGCCTTCCTTGACCCTGGAAGGTGCCACTCCCACTGT |
| | CCTTTCCTAATAAAATGAGGAAATTGCATCACATTGTCTGAGTAGGT |
| | GTCATTCTATTCTGGGGGGTGGGGTGGGGCAGGACAGCAAGGGGGA |
| | GGATTGGGAAGACAATAGCAGGCATGCTGGGGATGCAGTGGGCTCT |
| | ATGGCTTCTGAGGCAGAAAGAACCAGCTGGGGCTCGAGATCCACTAG |
| | GGCCGCAGGAACCCCTAGTGATGGAGTTGGCCACTCCCTCTCTGCGC |
| | GCTCGCTCGCTCACTGAGGCCGGGCGACCAAAGGTCGCCCGACGCCC |
| | GGGCTTTGCCCGGGCGGCCTCAGTGAGCGAGCGAGCGCGCAGCTGCC |
| | TGCAGG (SEQ. ID. NO: 94) |
| pAAV_ApoE_ | CCTGCAGGCAGCTGCGCGCTCGCTCGCTCACTGAGGCCGCCCGGGCA |
| hAAT_ | AAGCCCGGGCGGCCTCAGTGAGCGAGCGAGCGCGCAGAGAGGGAGT |
| HBB2m1_sp7. | GGCCAACTCCATCACTAGGGGTTCCTGCGGCCTAGTAGGCTCAGAGG |
| GLAvar45v2_ | CACACAGGAGTTTCTGGGCTCACCCTGCCCCCTTCCAACCCCTCAGTT |
| BGH228 | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGCTCCTGGGCAACCTGCTGGTCTCTC |
| | TGCTGGCCCATCACTTTGGCAAAGCAGCCACCATGGCCTTTCTGTGGC |
| | TGCTGTCCTGCTGGGCCCTGCTGGGGACCACCTTTGGCCTGGACAAT |
| | GGCCTGGCCAGGACCCCCACCATGGGCTGGCTGCACTGGGAGAGATT |
| | CATGTGCAACCTGGACTGCCAGGAGGAGCCTGACAGCTGCATCTCTG |
| | AGAAGCTGTTCATGGAGATGGCAGAGCTGATGGTGTCTGAGGGCTGG |
| | AAGGATGCTGGCTATGAGTACCTGTGCATTGATGACTGCTGGATGGC |
| | CCCCCAGAGAGACTCTGAGGGCAGGCTGCAGGCAGACCCCCAGAGG |
| | TTCCCCCATGGCATCAGACAGCTGGCCAACTATGTGCACAGCAAGGG |
| | CCTGAAGCTGGGCATCTATGCTGATGTGGGCAACAAGACCTGTGCAG |
| | GCTTCCCTGGCTCCTTTGGCTACTATGACATTGATGCCCAGACCTTTG |
| | CTGACTGGGGAGTGGACCTGCTGAAGTTTGATGGCTGCTACTGTGAC |
| | TCCCTGGAGAACCTGGCTGATGGCTACAAGCACATGTCCCTGGCCCT |
| | GAACAGGACAGGCAGATCCATTGTGTACAGCTGTGAGTGGCCCCTGT |
| | ACATGTGGCCATTCCAGAAGCCCAACTACACAGAGATCAGGCAGTAC |
| | TGCAACCACTGGAGAAACTTTGCAGACATTGATGATTCCTGGAAGAG |
| | CATCAAGTCCATCCTGGACTGGACCTCCTTCAACCAGGAGAGAATTG |
| | TGGATGTGGCAGGCCCTGGGGGCTGGAATGACCCAGACATGCTGGTC |
| | ATTGGCAACTTTGGCCTGTCCTGGAACCAGCAGGTGACCCAGATGGC |
| | CCTGTGGGCCATCATGGCAGCCCCCCTGTTCATGAGCAATGACCTGA |
| | GACACATCAGCCCACAGGCCAAGGCCCTGCTGCAGGACAAGGATGT |
| | GATTGCCATCAACCAGGACCCTCTGGGCAAGCAGGGCTACCAGCTGA |
| | GGCAGGGAGACAACTTTGAGGTGTGGGAGAGGCCCCTGTCTGGCCTG |
| | GCCTGGGCAGTGGCCATGATCAACAGGCAGGAGATTGGAGGCCCCA |
| | GGAGCTACACCATTGCAGTGGCCAGCCTGGGCAAGGGGGTGGCCTGC |
| | AACCCAGCCTGCTTCATCACCCAGCTGCTGCCTGTGAAGAGGAAGCT |
| | GGGCTTCTATGAGTGGACCTCCAGACTGAGGAGCCACATCAACCCCA |
| | CAGGCACAGTGCTGCTGCAGCTGGAGAACACCATGCAGATGAGCCTG |
| | AAGGACCTGCTGTGAAGATCTAGAGCTGAATTCCTGCAGCCAGGGGG |
| | ATCAGCCTCTACTGTGCCTTCTAGTTGCCAGCCATCTGTTGTTTGCCC |
| | CTCCCCCTTGCCTTCCTTGACCCTGGAAGGTGCCACTCCCACTGTCCT |
| | TTCCTAATAAAATGAGGAAATTGCATCACATTGTCTGAGTAGGTGTC |
| | ATTCTATTCTGGGGGGTGGGGTGGGGCAGGACAGCAAGGGGGAGGA |
| | TTGGGAAGACAATAGCAGGCATGCTGGGGATGCAGTGGGCTCTATGG |
| | CTTCTGAGGCAGAAAGAACCAGCTGGGGCTCGAGATCCACTAGGGCC |
| | GCAGGAACCCCTAGTGATGGAGTTGGCCACTCCCTCTCTGCGCGCTC |
| | GCTCGCTCACTGAGGCCGGGCGACCAAAGGTCGCCCGACGCCCGGGC |
| | TTTGCCCGGGCGGCCTCAGTGAGCGAGCGAGCGCGCAGCTGCCTGCA |
| | GG (SEQ. ID. NO: 95) |
| AAV_ApoE_ | CCTGCAGGCAGCTGCGCGCTCGCTCGCTCACTGAGGCCGCCCGGGCA |
| HAT_sp7_ | AAGCCCGGGCGGCCTCAGTGAGCGAGCGAGCGCGCAGAGAGGGAGT |
| GLA_WT_ | GGCCAACTCCATCACTAGGGGTTCCTGCGGCCTAGTAGGCTCAGAGG |
| intronIgHA | CACACAGGAGTTTCTGGGCTCACCCTGCCCCCTTCCAACCCCTCAGTT |
| | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGGTCCTGGGCAACCTGCTGGTCTCTC |
| | TGCTGGCCCATCACTTTGGCAAAGCACGCGTGCCACCATGGCCTTTCT |
| | GTGGCTGCTGTCCTGCTGGGCCCTGCTGGGGACCACCTTTGGCCTGG |
| | ACAATGGATTGGCAAGGACGCCTACCATGGGCTGGCTGCACTGGGAG |
| | CGCTTCATGTGCAACCTTGACTGCCAGGTGAGTACAGGAGGTGGAGA |
| | GTGGCCAGCCCTTCTCATGTTCAGAGAACATGGTTAACTGGTTAAGT |
| | CATGTCGTCCCACAGGAAGAGCCAGATTCCTGCATCAGTGAGAAGCT |
| | CTTCATGGAGATGGCAGAGCTCATGGTCTCAGAAGGCTGGAAGGATG |
| | CAGGTTATGAGTACCTCTGCATTGATGACTGTTGGATGGCTCCCCAA |
| | AGAGATTCAGAAGGCAGACTTCAGGCAGACCCTCAGCGCTTTCCTCA |
| | TGGGATTCGCCAGCTAGCTAATTATGTTCACAGCAAAGGACTGAAGC |
| | TAGGGATTTATGCAGATGTTGGAAATAAAACCTGCGCAGGCTTCCCT |
| | GGGAGTTTTGGATACTACGACATTGATGCCCAGACCTTTGCTGACTG |
| | GGGAGTTGATCTGCTAAAATTTGATGGTTGTTACTGTGACAGTTTGGA |
| | AAATTTGGCAGATGGTTATAAGCACATGTCCTTGGCCCTGAATAGGA |
| | CTGGCAGAAGCATTGTGTACTCCTGTGAGTGGCCTCTTTATATGTGGC |
| | CCTTTCAAAAGCCCAATTATACAGAAATCCGACAGTACTGCAATCAC |
| | TGGCGAAATTTTGCTGACATTGATGATTCCTGGAAAAGTATAAAGAG |
| | TATCTTGGACTGGACATCTTTTAACCAGGAGAGAATTGTTGATGTTGC |
| | TGGACCAGGGGGTTGGAATGACCCAGATATGTTAGTGATTGGCAACT |
| | TTGGCCTCAGCTGGAATCAGCAAGTAACTCAGATGGCCCTCTGGGCT |
| | ATCATGGCTGCTCCTTTATTCATGTCTAATGACCTCCGACACATCAGC |
| | CCTCAAGCCAAAGCTCTCCTTCAGGATAAGGACGTAATTGCCATCAA |
| | TCAGGACCCCTTGGGCAAGCAAGGGTACCAGCTTAGACAGGGAGAC |
| | AACTTTGAAGTGTGGGAACGACCTCTCTCAGGCTTAGCCTGGGCTGT |
| | AGCTATGATAAACCGGCAGGAGATTGGTGGACCTCGCTCTTATACCA |
| | TCGCAGTTGCTTCCCTGGGTAAAGGAGTGGCCTGTAATCCTGCCTGCT |
| | TCATCACACAGCTCCTCCCTGTGAAAAGGAAGCTAGGGTTCTATGAA |
| | TGGACTTCAAGGTTAAGAAGTCACATAAATCCCACAGGCACTGTTTT |
| | GCTTCAGCTAGAAAATACAATGCAGATGTCATTAAAAGACTTACTTT |
| | GAAGATCTAGAGCTGAATTCCTGCAGCCAGGGGGATCAGCCTCTACT |
| | GTGCCTTCTAGTTGCCAGCCATCTGTTGTTTGCCCCTCCCCCTTGCCTT |
| | CCTTGACCCTGGAAGGTGCCACTCCCACTGTCCTTTCCTAATAAAATG |
| | AGGAAATTGCATCACATTGTCTGAGTAGGTGTCATTCTATTCTGGGG |
| | GGTGGGGTGGGGCAGGACAGCAAGGGGGAGGATTGGGAAGACAATA |
| | GCAGGCATGCTGGGGATGCAGTGGGCTCTATGGCTTCTGAGGCAGAA |
| | AGAACCAGCTGGGGCTCGAGATCCACTAGGGCCGCAGGAACCCCTA |
| | GTGATGGAGTTGGCCACTCCCTCTCTGCGCGCTCGCTCGCTCACTGAG |
| | GCCGGGCGACCAAAGGTCGCCCGACGCCCGGGCTTTGCCCGGGCGGC |
| | CTCAGTGAGCGAGCGAGCGCGCAGCTGCCTGCAGG (SEQ. ID. NO: 96) |
| AAV_ApoE_ | CCTGCAGGCAGCTGCGCGCTCGCTCGCTCACTGAGGCCGCCCGGGCA |
| HAT_sp7_ | AAGCCCGGGCGGCCTCAGTGAGCGAGCGAGCGCGCAGAGAGGGAGT |
| GLA_WT_ | GGCCAACTCCATCACTAGGGGTTCCTGCGGCCTAGTAGGCTCAGAGG |
| intronIgHmu | CACACAGGAGTTTCTGGGCTCACCCTGCCCCCTTCCAACCCCTCAGTT |
| | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGGTCCTGGGCAACCTGCTGGTCTCTC |
| | TGCTGGCCCATCACTTTGGCAAAGCACGCGTGCCACCATGGCCTTTCT |
| | GTGGCTGCTGTCCTGCTGGGCCCTGCTGGGGACCACCTTTGGCCTGG |
| | ACAATGGATTGGCAAGGACGCCTACCATGGGCTGGCTGCACTGGGAG |
| | CGCTTCATGTGCAACCTTGACTGCCAGGTAAGAACCAAACCCTCCCA |
| | GCAGGGGTGCCCAGGCCCAGGCATGGCCCAGAGGGAGCAGCGGGTG |
| | GGGCTTAGGCCAAGCTGAGCTCACACCTTGACCTTTCATTACAGGAA |
| | GAGCCAGATTCCTGCATCAGTGAGAAGCTCTTCATGGAGATGGCAGA |
| | GCTCATGGTCTCAGAAGGCTGGAAGGATGCAGGTTATGAGTACCTCT |
| | GCATTGATGACTGTTGGATGGCTCCCCAAAGAGATTCAGAAGGCAGA |
| | CTTCAGGCAGACCCTCAGCGCTTTCCTCATGGGATTCGCCAGCTAGCT |
| | AATTATGTTCACAGCAAAGGACTGAAGCTAGGGATTTATGCAGATGT |
| | TGGAAATAAAACCTGCGCAGGCTTCCCTGGGAGTTTTGGATACTACG |
| | ACATTGATGCCCAGACCTTTGCTGACTGGGGAGTTGATCTGCTAAAA |
| | TTTGATGGTTGTTACTGTGACAGTTTGGAAAATTTGGCAGATGGTTAT |
| | AAGCACATGTCCTTGGCCCTGAATAGGACTGGCAGAAGCATTGTGTA |
| | CTCCTGTGAGTGGCCTCTTTATATGTGGCCCTTTCAAAAGCCCAATTA |
| | TACAGAAATCCGACAGTACTGCAATCACTGGCGAAATTTTGCTGACA |
| | TTGATGATTCCTGGAAAAGTATAAAGAGTATCTTGGACTGGACATCT |
| | TTTAACCAGGAGAGAATTGTTGATGTTGCTGGACCAGGGGGTTGGAA |
| | TGACCCAGATATGTTAGTGATTGGCAACTTTGGCCTCAGCTGGAATC |
| | AGCAAGTAACTCAGATGGCCCTCTGGGCTATCATGGCTGCTCCTTTAT |
| | TCATGTCTAATGACCTCCGACACATCAGCCCTCAAGCCAAAGCTCTC |
| | CTTCAGGATAAGGACGTAATTGCCATCAATCAGGACCCCTTGGGCAA |
| | GCAAGGGTACCAGCTTAGACAGGGAGACAACTTTGAAGTGTGGGAA |
| | CGACCTCTCTCAGGCTTAGCCTGGGCTGTAGCTATGATAAACCGGCA |
| | GGAGATTGGTGGACCTCGCTCTTATACCATCGCAGTTGCTTCCCTGGG |
| | TAAAGGAGTGGCCTGTAATCCTGCCTGCTTCATCACACAGCTCCTCCC |
| | TGTGAAAAGGAAGCTAGGGTTCTATGAATGGACTTCAAGGTTAAGAA |
| | GTCACATAAATCCCACAGGCACTGTTTTGCTTCAGCTAGAAAATACA |
| | ATGCAGATGTCATTAAAAGACTTACTTTGAAGATCTAGAGCTGAATT |
| | CCTGCAGCCAGGGGGATCAGCCTCTACTGTGCCTTCTAGTTGCCAGC |
| | CATCTGTTGTTTGCCCCTCCCCCTTGCCTTCCTTGACCCTGGAAGGTG |
| | CCACTCCCACTGTCCTTTCCTAATAAAATGAGGAAATTGCATCACATT |
| | GTCTGAGTAGGTGTCATTCTATTCTGGGGGGTGGGGTGGGGCAGGAC |
| | AGCAAGGGGGAGGATTGGGAAGACAATAGCAGGCATGCTGGGGATG |
| | CAGTGGGCTCTATGGCTTCTGAGGCAGAAAGAACCAGCTGGGGCTCG |
| | AGATCCACTAGGGCCGCAGGAACCCCTAGTGATGGAGTTGGCCACTC |
| | CCTCTCTGCGCGCTCGCTCGCTCACTGAGGCCGGGCGACCAAAGGTC |
| | GCCCGACGCCCGGGCTTTGCCCGGGCGGCCTCAGTGAGCGAGCGAGC |
| | GCGCAGCTGCCTGCAGG (SEQ. ID. NO: 97) |
| AAV_ApoE_ | CCTGCAGGCAGCTGCGCGCTCGCTCGCTCACTGAGGCCGCCCGGGCA |
| HAT_sp7_ | AAGCCCGGGCGGCCTCAGTGAGCGAGCGAGCGCGCAGAGAGGGAGT |
| GLA_WT_ | GGCCAACTCCATCACTAGGGGTTCCTGCGGCCTAGTAGGCTCAGAGG |
| intronRBP4 | CACACAGGAGTTTCTGGGCTCACCCTGCCCCCTTCCAACCCCTCAGTT |
| | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGGTCCTGGGCAACCTGCTGGTCTCTC |
| | TGCTGGCCCATCACTTTGGCAAAGCACGCGTGCCACCATGGCCTTTCT |
| | GTGGCTGCTGTCCTGCTGGGCCCTGCTGGGGACCACCTTTGGCCTGG |
| | ACAATGGATTGGCAAGGACGCCTACCATGGGCTGGCTGCACTGGGAG |
| | CGCTTCATGTGCAACCTTGACTGCCAGGTCAGTGGCCGCCGGGGCAG |
| | CTGCGCCCTTTGCGCTCCAGGGTTCCCCAAGGGCCCTGCCTGCTGATC |
| | GCCACGTGGGCATTGTGAAGGGAAGGGAGCACCGAATGGGTGGAGG |
| | GAGGGAGGAAGCCCTTTGCCCGGCTTGGCTGAGGATCCCCTTGGCTT |
| | TTGCAGGAAGAGCCAGATTCCTGCATCAGTGAGAAGCTCTTCATGGA |
| | GATGGCAGAGCTCATGGTCTCAGAAGGCTGGAAGGATGCAGGTTATG |
| | AGTACCTCTGCATTGATGACTGTTGGATGGCTCCCCAAAGAGATTCA |
| | GAAGGCAGACTTCAGGCAGACCCTCAGCGCTTTCCTCATGGGATTCG |
| | CCAGCTAGCTAATTATGTTCACAGCAAAGGACTGAAGCTAGGGATTT |
| | ATGCAGATGTTGGAAATAAAACCTGCGCAGGCTTCCCTGGGAGTTTT |
| | GGATACTACGACATTGATGCCCAGACCTTTGCTGACTGGGGAGTTGA |
| | TCTGCTAAAATTTGATGGTTGTTACTGTGACAGTTTGGAAAATTTGGC |
| | AGATGGTTATAAGCACATGTCCTTGGCCCTGAATAGGACTGGCAGAA |
| | GCATTGTGTACTCCTGTGAGTGGCCTCTTTATATGTGGCCCTTTCAAA |
| | AGCCCAATTATACAGAAATCCGACAGTACTGCAATCACTGGCGAAAT |
| | TTTGCTGACATTGATGATTCCTGGAAAAGTATAAAGAGTATCTTGGA |
| | CTGGACATCTTTTAACCAGGAGAGAATTGTTGATGTTGCTGGACCAG |
| | GGGGTTGGAATGACCCAGATATGTTAGTGATTGGCAACTTTGGCCTC |
| | AGCTGGAATCAGCAAGTAACTCAGATGGCCCTCTGGGCTATCATGGC |
| | TGCTCCTTTATTCATGTCTAATGACCTCCGACACATCAGCCCTCAAGC |
| | CAAAGCTCTCCTTCAGGATAAGGACGTAATTGCCATCAATCAGGACC |
| | CCTTGGGCAAGCAAGGGTACCAGCTTAGACAGGGAGACAACTTTGA |
| | AGTGTGGGAACGACCTCTCTCAGGCTTAGCCTGGGCTGTAGCTATGA |
| | TAAACCGGCAGGAGATTGGTGGACCTCGCTCTTATACCATCGCAGTT |
| | GCTTCCCTGGGTAAAGGAGTGGCCTGTAATCCTGCCTGCTTCATCACA |
| | CAGCTCCTCCCTGTGAAAAGGAAGCTAGGGTTCTATGAATGGACTTC |
| | AAGGTTAAGAAGTCACATAAATCCCACAGGCACTGTTTTGCTTCAGC |
| | TAGAAAATACAATGCAGATGTCATTAAAAGACTTACTTTGAAGATCT |
| | AGAGCTGAATTCCTGCAGCCAGGGGGATCAGCCTCTACTGTGCCTTC |
| | TAGTTGCCAGCCATCTGTTGTTTGCCCCTCCCCCTTGCCTTCCTTGACC |
| | CTGGAAGGTGCCACTCCCACTGTCCTTTCCTAATAAAATGAGGAAAT |
| | TGCATCACATTGTCTGAGTAGGTGTCATTCTATTCTGGGGGGTGGGGT |
| | GGGGCAGGACAGCAAGGGGGAGGATTGGGAAGACAATAGCAGGCAT |
| | GCTGGGGATGCAGTGGGCTCTATGGCTTCTGAGGCAGAAAGAACCAG |
| | CTGGGGCTCGAGATCCACTAGGGCCGCAGGAACCCCTAGTGATGGAG |
| | TTGGCCACTCCCTCTCTGCGCGCTCGCTCGCTCACTGAGGCCGGGCGA |
| | CCAAAGGTCGCCCGACGCCCGGGCTTTGCCCGGGCGGCCTCAGTGAG |
| | CGAGCGAGCGCGCAGCTGCCTGCAGG (SEQ. ID. NO: 98) |
| AAV_ApoE_ | CCTGCAGGCAGCTGCGCGCTCGCTCGCTCACTGAGGCCGCCCGGGCA |
| HAT_sp7_ | AAGCCCGGGCGGCCTCAGTGAGCGAGCGAGCGCGCAGAGAGGGAGT |
| GLA_WT_ | GGCCAACTCCATCACTAGGGGTTCCTGCGGCCTAGTAGGCTCAGAGG |
| intronVTN1 | CACACAGGAGTTTCTGGGCTCACCCTGCCCCCTTCCAACCCCTCAGTT |
| | CCCATCCTCCAGCAGCTGTTTGTGTGCTGCCTCTGAAGTCCACACTGA |
| | ACAAACTTCAGCCTACTCATGTCCCTAAAATGGGCAAACATTGCAAG |
| | CAGCAAACAGCAAACACACAGCCCTCCCTGCCTGCTGACCTTGGAGC |
| | TGGGGCAGAGGTCAGAGACCTCTCTGGGCCCATGCCACCTCCAACAT |
| | CCACTCGACCCCTTGGAATTTCGGTGGAGAGGAGCAGAGGTTGTCCT |
| | GGCGTGGTTTAGGTAGTGTGAGAGGGGTACCCGGGGATCTTGCTACC |
| | AGTGGAACAGCCACTAAGGATTCTGCAGTGAGAGCAGAGGGCCAGC |
| | TAAGTGGTACTCTCCCAGAGACTGTCTGACTCACGCCACCCCCTCCAC |
| | CTTGGACACAGGACGCTGTGGTTTCTGAGCCAGGTACAATGACTCCT |
| | TTCGGTAAGTGCAGTGGAAGCTGTACACTGCCCAGGCAAAGCGTCCG |
| | GGCAGCGTAGGCGGGCGACTCAGATCCCAGCCAGTGGACTTAGCCCC |
| | TGTTTGCTCCTCCGATAACTGGGGTGACCTTGGTTAATATTCACCAGC |
| | AGCCTCCCCCGTTGCCCCTCTGGATCCACTGCTTAAATACGGACGAG |
| | GACAGGGCCCTGTCTCCTCAGCTTCAGGCACCACCACTGACCTGGGA |
| | CAGTGAATAGATCCTGAGAACTTCAGGGTGAGTCTATGGGACCCTTG |
| | ATGTTTTCTTTCCCCTTCTTTTCTATGGTTAAGTTCATGTCATAGGAAG |
| | GGGAGAAGTAACAGGGTACACATATTGACCAAATCAGGGTAATTTTG |
| | CATTTGTAATTTTAAAAAATGCTTTCTTCTTTTAATATACTTTTTTGTT |
| | TATCTTATTTCTAATACTTTCCCTAATCTCTTTCTTTCAGGGCAATAAT |
| | GATACAATGTATCATGCCTCTTTGCACCATTCTAAAGAATAACAGTG |
| | ATAATTTCTGGGTTAAGGCAATAGCAATATTTCTGCATATAAATATTT |
| | CTGCATATAAATTGTAACTGATGTAAGAGGTTTCATATTGCTAATAGC |
| | AGCTACAATCCAGCTACCATTCTGCTTTTATTTTCTGGTTGGGATAAG |
| | GCTGGATTATTCTGAGTCCAAGCTAGGCCCTTTTGCTAATCTTGTTCA |
| | TACCTCTTATCTTCCTCCCACAGGTCCTGGGCAACCTGCTGGTCTCTC |
| | TGCTGGCCCATCACTTTGGCAAAGCACGCGTGCCACCATGGCCTTTCT |
| | GTGGCTGCTGTCCTGCTGGGCCCTGCTGGGGACCACCTTTGGCCTGG |
| | ACAATGGATTGGCAAGGACGCCTACCATGGGCTGGCTGCACTGGGAG |
| | CGCTTCATGTGCAACCTTGACTGCCAGGTATGGAGAGAGGGCAAGTC |
| | TTGCTTCTCCCTCAAAAGGGCTGAAACCCCTTGGTATTGGTAGAGCC |
| | AGGCCGGCTGGAGGGGGCTGTGGTTGTGGAGCTATCGATCAAAGTCT |
| | GTTTGCTCAGGCCAGACTTTGCTTCTGTTGACCTTTTGGGGAAAGCTC |
| | AGCTCTACCTGGACCCCACACCTTGGACTTTGCCTAGCACAGCTGAG |
| | AGCACAGCCAGCAGAGGGAGGGGCTGTGGCTGAGGAGTTTAGGGGG |
| | CCTGGGGGGGTGGGGTCGAGACACCAGTGATATGGTGGAGGGAAAG |
| | CACAGGGGGAAGGGAATTGGACTGAGAGTCAAAGGCCTGGCTCTGC |
| | CATTCGCTGCTGTGTGTCTTTGGGCAAGGTGCAGCAGATGAACTCTA |
| | ATGGCCCCGCTGGAAGGGGCAAGATTCGGACCCCCAAGACCTCTCAT |
| | TCACCCCTTCCCTGCCACAGGAAGAGCCAGATTCCTGCATCAGTGAG |
| | AAGCTCTTCATGGAGATGGCAGAGCTCATGGTCTCAGAAGGCTGGAA |
| | GGATGCAGGTTATGAGTACCTCTGCATTGATGACTGTTGGATGGCTC |
| | CCCAAAGAGATTCAGAAGGCAGACTTCAGGCAGACCCTCAGCGCTTT |
| | CCTCATGGGATTCGCCAGCTAGCTAATTATGTTCACAGCAAAGGACT |
| | GAAGCTAGGGATTTATGCAGATGTTGGAAATAAAACCTGCGCAGGCT |
| | TCCCTGGGAGTTTTGGATACTACGACATTGATGCCCAGACCTTTGCTG |
| | ACTGGGGAGTTGATCTGCTAAAATTTGATGGTTGTTACTGTGACAGTT |
| | TGGAAAATTTGGCAGATGGTTATAAGCACATGTCCTTGGCCCTGAAT |
| | AGGACTGGCAGAAGCATTGTGTACTCCTGTGAGTGGCCTCTTTATAT |
| | GTGGCCCTTTCAAAAGCCCAATTATACAGAAATCCGACAGTACTGCA |
| | ATCACTGGCGAAATTTTGCTGACATTGATGATTCCTGGAAAAGTATA |
| | AAGAGTATCTTGGACTGGACATCTTTTAACCAGGAGAGAATTGTTGA |
| | TGTTGCTGGACCAGGGGGTTGGAATGACCCAGATATGTTAGTGATTG |
| | GCAACTTTGGCCTCAGCTGGAATCAGCAAGTAACTCAGATGGCCCTC |
| | TGGGCTATCATGGCTGCTCCTTTATTCATGTCTAATGACCTCCGACAC |
| | ATCAGCCCTCAAGCCAAAGCTCTCCTTCAGGATAAGGACGTAATTGC |
| | CATCAATCAGGACCCCTTGGGCAAGCAAGGGTACCAGCTTAGACAGG |
| | GAGACAACTTTGAAGTGTGGGAACGACCTCTCTCAGGCTTAGCCTGG |
| | GCTGTAGCTATGATAAACCGGCAGGAGATTGGTGGACCTCGCTCTTA |
| | TACCATCGCAGTTGCTTCCCTGGGTAAAGGAGTGGCCTGTAATCCTG |
| | CCTGCTTCATCACACAGCTCCTCCCTGTGAAAAGGAAGCTAGGGTTC |
| | TATGAATGGACTTCAAGGTTAAGAAGTCACATAAATCCCACAGGCAC |
| | TGTTTTGCTTCAGCTAGAAAATACAATGCAGATGTCATTAAAAGACT |
| | TACTTTGAAGATCTAGAGCTGAATTCCTGCAGCCAGGGGGATCAGCC |
| | TCTACTGTGCCTTCTAGTTGCCAGCCATCTGTTGTTTGCCCCTCCCCCT |
| | TGCCTTCCTTGACCCTGGAAGGTGCCACTCCCACTGTCCTTTCCTAAT |
| | AAAATGAGGAAATTGCATCACATTGTCTGAGTAGGTGTCATTCTATT |
| | CTGGGGGGTGGGGTGGGGCAGGACAGCAAGGGGGAGGATTGGGAAG |
| | ACAATAGCAGGCATGCTGGGGATGCAGTGGGCTCTATGGCTTCTGAG |
| | GCAGAAAGAACCAGCTGGGGCTCGAGATCCACTAGGGCCGCAGGAA |
| | CCCCTAGTGATGGAGTTGGCCACTCCCTCTCTGCGCGCTCGCTCGCTC |
| | ACTGAGGCCGGGCGACCAAAGGTCGCCCGACGCCCGGGCTTTGCCCG |
| | GGCGGCCTCAGTGAGCGAGCGAGCGCGCAGCTGCCTGCAGG (SEQ. |
| | ID. NO: 99) |
| GLA | LDNGLARTPTMGWLHWERFMCNLDCQEEPDSCISEKLFMEMAELMVSE |
| | GWKDAGYEYLCIDDCWMAPQRDSEGRLQADPQRFPHGIRQLANYVHS |
| | KGLKLGIYADVGNKTCAGFPGSFGYYDIDAQTFADWGVDLLKFDGCYC |
| | DSLENLADGYKHMSLALNRTGRSIVYSCEWPLYMWPFQKPNYTEIRQY |
| | CNHWRNFADIDDSWKSIKSILDWTSFNQERIVDVAGPGGWNDPDMLVI |
| | GNFGLSWNQQVTQMALWAIMAAPLFMSNDLRHISPQAKALLQDKDVIA |
| | INQDPLGKQGYQLRQGDNFEVWERPLSGLAWAVAMINRQEIGGPRSYTI |
| | AVASLGKGVACNPACFITQLLPVKRKLGFYEWTSRLRSHINPTGTVLLQ |
| | LENTMQMSLKDLL (SEQ ID NO: 100) |
| sp7-GLA | MAFLWLLSCWALLGTTFGLDNGLARTPTMGWLHWERFMCNLDCQEEP |
| | DSCISEKLFMEMAELMVSEGWKDAGYEYLCIDDCWMAPQRDSEGRLQ |
| | ADPQRFPHGIRQLANYVHSKGLKLGIYADVGNKTCAGFPGSFGYYDIDA |
| | QTFADWGVDLLKFDGCYCDSLENLADGYKHMSLALNRTGRSIVYSCE |
| | WPLYMWPFQKPNYTEIRQYCNHWRNFADIDDSWKSIKSILDWTSFNQE |
| | RIVDVAGPGGWNDPDMLVIGNFGLSWNQQVTQMALWAIMAAPLFMSN |
| | DLRHISPQAKALLQDKDVIAINQDPLGKQGYQLRQGDNFEVWERPLSGL |
| | AWAVAMINRQEIGGPRSYTIAVASLGKGVACNPACFITQLLPVKRKLGF |
| | YEWTSRLRSHINPTGTVLLQLENTMQMSLKDLL (SEQ ID NO: 101) |
| spAHSG- | MKSLVLLLCLAQLWGCHSLDNGLARTPTMGWLHWERFMCNLDCQEEP |
| GLA | DSCISEKLFMEMAELMVSEGWKDAGYEYLCIDDCWMAPQRDSEGRLQ |
| | ADPQRFPHGIRQLANYVHSKGLKLGIYADVGNKTCAGFPGSFGYYDIDA |
| | QTFADWGVDLLKFDGCYCDSLENLADGYKHMSLALNRTGRSIVYSCE |
| | WPLYMWPFQKPNYTEIRQYCNHWRNFADIDDSWKSIKSILDWTSFNQE |
| | RIVDVAGPGGWNDPDMLVIGNFGLSWNQQVTQMALWAIMAAPLFMSN |
| | DLRHISPQAKALLQDKDVIAINQDPLGKQGYQLRQGDNFEVWERPLSGL |
| | AWAVAMINRQEIGGPRSYTIAVASLGKGVACNPACFITQLLPVKRKLGF |
| | YEWTSRLRSHINPTGTVLLQLENTMQMSLKDLL (SEQ ID NO: 102) |
| spCD300- | MWLPWALLLLWVPGCFALDNGLARTPTMGWLHWERFMCNLDCQEEP |
| GLA | DSCISEKLFMEMAELMVSEGWKDAGYEYLCIDDCWMAPQRDSEGRLQ |
| | ADPQRFPHGIRQLANYVHSKGLKLGIYADVGNKTCAGFPGSFGYYDIDA |
| | QTFADWGVDLLKFDGCYCDSLENLADGYKHMSLALNRTGRSIVYSCE |
| | WPLYMWPFQKPNYTEIRQYCNHWRNFADIDDSWKSIKSILDWTSFNQE |
| | RIVDVAGPGGWNDPDMLVIGNFGLSWNQQVTQMALWAIMAAPLFMSN |
| | DLRHISPQAKALLQDKDVIAINQDPLGKQGYQLRQGDNFEVWERPLSGL |
| | AWAVAMINRQEIGGPRSYTIAVASLGKGVACNPACFITQLLPVKRKLGF |
| | YEWTSRLRSHINPTGTVLLQLENTMQMSLKDLL (SEQ ID NO: 103) |
| spGLA- | MQLRNPELHLGCALALRFLALVSWDIPGARALDNGLARTPTMGWLHW |
| GLA | ERFMCNLDCQEEPDSCISEKLFMEMAELMVSEGWKDAGYEYLCIDDCW |
| | MAPQRDSEGRLQADPQRFPHGIRQLANYVHSKGLKLGIYADVGNKTCA |
| | GFPGSFGYYDIDAQTFADWGVDLLKFDGCYCDSLENLADGYKHMSLAL |
| | NRTGRSIVYSCEWPLYMWPFQKPNYTEIRQYCNHWRNFADIDDSWKSI |
| | KSILDWTSFNQERIVDVAGPGGWNDPDMLVIGNFGLSWNQQVTQMAL |
| | WAIMAAPLFMSNDLRHISPQAKALLQDKDVIAINQDPLGKQGYQLRQG |
| | DNFEVWERPLSGLAWAVAMINRQEIGGPRSYTIAVASLGKGVACNPACF |
| | ITQLLPVKRKLGFYEWTSRLRSHINPTGTVLLQLENTMQMSLKDLL (SEQ |
| | ID NO: 104) |
| spLAMP1- | MAAPGSARRPLLLLLLLLLLGLMHCASALDNGLARTPTMGWLHWERF |
| GLA aa | MCNLDCQEEPDSCISEKLFMEMAELMVSEGWKDAGYEYLCIDDCWMA |
| | PQRDSEGRLQADPQRFPHGIRQLANYVHSKGLKLGIYADVGNKTCAGFP |
| | GSFGYYDIDAQTFADWGVDLLKFDGCYCDSLENLADGYKHMSLALNR |
| | TGRSIVYSCEWPLYMWPFQKPNYTEIRQYCNHWRNFADIDDSWKSIKSI |
| | LDWTSFNQERIVDVAGPGGWNDPDMLVIGNFGLSWNQQVTQMALWAI |
| | MAAPLFMSNDLRHISPQAKALLQDKDVIAINQDPLGKQGYQLRQGDNF |
| | EVWERPLSGLAWAVAMINRQEIGGPRSYTIAVASLGKGVACNPACFITQ |
| | LLPVKRKLGFYEWTSRLRSHINPTGTVLLQLENTMQMSLKDLL (SEQ ID |
| | NO: 105) |
| spNotch2- | MPALRPALLWALLALWLCCAAPAHALDNGLARTPTMGWLHWERFMC |
| GLA | NLDCQEEPDSCISEKLFMEMAELMVSEGWKDAGYEYLCIDDCWMAPQ |
| | RDSEGRLQADPQRFPHGIRQLANYVHSKGLKLGIYADVGNKTCAGFPGS |
| | FGYYDIDAQTFADWGVDLLKFDGCYCDSLENLADGYKHMSLALNRTG |
| | RSIVYSCEWPLYMWPFQKPNYTEIRQYCNHWRNFADIDDSWKSIKSILD |
| | WTSFNQERIVDVAGPGGWNDPDMLVIGNFGLSWNQQVTQMALWAIMA |
| | APLFMSNDLRHISPQAKALLQDKDVIAINQDPLGKQGYQLRQGDNFEV |
| | WERPLSGLAWAVAMINRQEIGGPRSYTIAVASLGKGVACNPACFITQLL |
| | PVKRKLGFYEWTSRLRSHINPTGTVLLQLENTMQMSLKDLL (SEQ ID |
| | NO: 106) |
| spORM1- | MALSWVLTVLSLLPLLEALDNGLARTPTMGWLHWERFMCNLDCQEEP |
| GLA | DSCISEKLFMEMAELMVSEGWKDAGYEYLCIDDCWMAPQRDSEGRLQ |
| | ADPQRFPHGIRQLANYVHSKGLKLGIYADVGNKTCAGFPGSFGYYDIDA |
| | QTFADWGVDLLKFDGCYCDSLENLADGYKHMSLALNRTGRSIVYSCE |
| | WPLYMWPFQKPNYTEIRQYCNHWRNFADIDDSWKSIKSILDWTSFNQE |
| | RIVDVAGPGGWNDPDMLVIGNFGLSWNQQVTQMALWAIMAAPLFMSN |
| | DLRHISPQAKALLQDKDVIAINQDPLGKQGYQLRQGDNFEVWERPLSGL |
| | AWAVAMINRQEIGGPRSYTIAVASLGKGVACNPACFITQLLPVKRKLGF |
| | YEWTSRLRSHINPTGTVLLQLENTMQMSLKDLL (SEQ ID NO: 107) |
| spTF-GLA | MRLAVGALLVCAVLGLCLALDNGLARTPTMGWLHWERFMCNLDCQE |
| | EPDSCISEKLFMEMAELMVSEGWKDAGYEYLCIDDCWMAPQRDSEGRL |
| | QADPQRFPHGIRQLANYVHSKGLKLGIYADVGNKTCAGFPGSFGYYDID |
| | AQTFADWGVDLLKFDGCYCDSLENLADGYKHMSLALNRTGRSIVYSCE |
| | WPLYMWPFQKPNYTEIRQYCNHWRNFADIDDSWKSIKSILDWTSFNQE |
| | RIVDVAGPGGWNDPDMLVIGNFGLSWNQQVTQMALWAIMAAPLFMSN |
| | DLRHISPQAKALLQDKDVIAINQDPLGKQGYQLRQGDNFEVWERPLSGL |
| | AWAVAMINRQEIGGPRSYTIAVASLGKGVACNPACFITQLLPVKRKLGF |
| | YEWTSRLRSHINPTGTVLLQLENTMQMSLKDLL (SEQ ID NO: 108) |
| sp7-GLA 7 | MAFLWLLSCWALLGTTFGLDNGLARTPTLDNGLARTPTMGWLHWERF |
| mut | MCNLDCKEEPDSCISEKLFMEMAELMVSEGWKDAGYEYLCIDDCWMA |
| | PQRDSEGRLQADPERFPHGIRQLANYVHSKGLKLGIYADVGNKTCAGFP |
| | GSFGYYDIDAQTFADWGVDLLKFDGCYCDSLENLADGYKHMSLALNR |
| | TGRSIVYSCEWPLYMWPFQEPNYTEIRQYCNHWRNFADIDDSWQSIKSI |
| | LDWTSTNQERIVDVAGPGGWNDPDMLVIGNFGLSWNQQVTQMALWAI |
| | MAAPLFMSNDLRHISPQAKALLQDKDVIAINQDPLGKQGYQLRQEDNFE |
| | VWERPLSNLAWAVAMINRQEIGGPRSYTIAVASLGKGVACNPACFITQL |
| | LPVKRKLGFYEWTSRLRSHINPTGTVLLQLENTMQMSLKDLL (SEQ ID |
| | NO: 109) |

In Table 1, Termination codons for SEQ ID NOs: 15-19 are shown in bold.

### Additional Aspects and Embodiments

A first set of additional aspects and embodiments are provided by:
1. A polynucleotide comprising a nucleic acid encoding α-galactosidase A (GLA), wherein the nucleic acid is selected from the group consisting of: (1) a polynucleotide having at least 75% sequence identity to the sequence of SEQ ID NO: 15, (2) a polynucleotide having at least 84% sequence identity to the sequence of SEQ ID NO: 16, (3) a polynucleotide having at least 86% sequence identity to the sequence of SEQ ID NO: 17, (4) a polynucleotide having at least 86% sequence identity to the sequence of SEQ ID NO: 18, and (5) a polynucleotide having at least 83% sequence identity to the sequence of SEQ ID NO: 19, optionally, the GLA comprises the amino acid sequence of SEQ ID NO: 100.
2. The polynucleotide of 1, wherein the nucleic acid contains fewer than 14 CpG dinucleotides, optionally 0 CpG dinucleotides.
3. The polynucleotide of 1 or 2, wherein the nucleic acid has a sequence of any one of SEQ ID NOs: 15-19.
4. A polynucleotide comprising a nucleic acid encoding an α-galactosidase A (GLA) protein, wherein said GLA protein has an amino acid sequence of SEQ ID NO: 100 having one or more amino acid substitutions selected from the group consisting of Gln57Lys, Gln111Glu, Lys213Glu, Lys237Gln, Phe248Thr, Gly334Glu, and Gly346Asn, optionally, the GLA comprises the amino acid sequence of SEQ ID NO: 48.
5. The polynucleotide of 4, wherein the nucleic acid has a sequence of SEQ ID NO: 47.
6. The polynucleotide of any one of 1-5, further comprising an intron positioned within the nucleic acid encoding the GLA.
7. The polynucleotide of 6, wherein the intron is positioned between nucleotides 78 and 79 of the nucleic encoding the GLA, wherein the nucleotide positions are given in reference to the coding sequence of GLA having a sequence of SEQ ID NO: 14.
8. The polynucleotide of 7, wherein the intron is selected from the group consisting of an intron from a vitronectin1 (VTN1) gene, a retinol binding protein 4 (RBP4) gene, a mouse IgG heavy chain A (IgHA) gene, and a mouse IgG heavy chain μ (IgHμ) gene, optionally, the intron comprises a sequence of one of SEQ ID NOs: 49-52.
9. The polynucleotide of any one of 6-8, wherein the nucleic acid has a sequence of any one of SEQ ID NOs: 43-46.
10. The polynucleotide of any one of 1-9, further comprising a second nucleic acid that encodes a signal peptide sequence positioned at the 5′ end of the nucleic acid encoding the GLA.
11. The polynucleotide of 10, wherein the signal peptide sequence is a heterologous or an endogenous or native signal peptide sequence.
12. The polynucleotide of 10 or 11, wherein the signal peptide is selected from the group consisting of human chymotrypsinogen B2 signal peptide, AHSG signal peptide, CD300 signal peptide, LAMP1 signal peptide, Notch 2 signal peptide, ORM1 signal peptide, TF signal peptide, and wild-type GLA signal peptide, or a variant thereof.
13. The polynucleotide of 12, wherein the signal peptide is a human chymotrypsinogen B2 signal peptide.
14. The polynucleotide of 13, wherein the nucleic acid encoding the signal peptide has a sequence of any one of SEQ ID NOs: 1-5.
15. An expression cassette comprising the polynucleotide of any one of 1-14, operably linked to an expression control element.
16. The expression cassette of 15, wherein the expression control element is a liver-specific expression control element.
17. The expression cassette of 15 or 16, wherein the expression control element is positioned 5′ of the nucleic acid encoding GLA.
18. The expression cassette of any one of 15-17, further comprising a poly-adenylation sequence positioned 3′ of the nucleic acid encoding GLA.
19. The expression cassette of any one of 15-18, wherein the expression control element or poly-adenylation sequence is CpG-reduced compared to the wild-type expression control element or polyadenylation sequence.
20. The expression cassette of any one of 15-19, wherein the expression control element comprises an ApoE/hAAT enhancer/promoter sequence.
21. The expression cassette of any one of 15-20, wherein the poly-adenylation sequence comprises a bovine growth hormone (bGH) polyadenylation sequence.
22. The expression cassette of 20 or 21, wherein the ApoE/hAAT enhancer/promoter sequence or the bGH polyadenylation sequence is CpG-reduced compared to wild-type ApoE/hAAT enhancer/promoter sequence or bGH polyadenylation sequence.
23. The expression cassette of 20, wherein the ApoE/hAAT enhancer/promoter sequence comprises the sequence of SEQ ID NO: 38.
24. The expression cassette of 21, wherein the bGH polyadenylation sequence comprises the sequence of SEQ ID NO: 20.
25. The expression cassette of any one of 15-24, further comprising an intron positioned between the 3′ end of the expression control element and the 5′ end of the nucleic acid.
26. The expression cassette of 25, wherein the intron comprises an hBB2m1 intron.
27. The expression cassette of 26, wherein the intron sequence comprises the sequence of SEQ ID NO: 39.
28. An adeno-associated virus (AAV) vector comprising the polynucleotide or expression cassette of any one of 1-27.
29. The AAV vector of 28, wherein the AAV vector comprises: (a) one or more of an AAV capsid, and (b) one or more AAV inverted terminal repeats (ITRs), wherein the AAV ITR(s) flanks the 5′ or 3′ terminus of the nucleic acid or the expression cassette.
30. The AAV vector of 29, wherein at least one or more of the ITRs is modified to have reduced CpGs.
31. The AAV vector of any one of 28-30, wherein the AAV vector has a capsid serotype comprising a modified or variant AAV VP1, VP2 and/or VP3 capsid having 90% or more sequence identity to AAV1, AAV2, AAV3, AAV4, AAV5, AAV6, AAV7, AAV8, AAV9, AAV10, AAV 11, AAV12, Rh10, Rh74 (SEQ ID NO: 35), AAV3B, LK03 (SEQ ID NO: 42), AAV-2i8, SEQ ID NO: 110, SEQ ID NO: 36, and/or SEQ ID NO: 37; or a capsid having 95% or more sequence identity to AAV1, AAV2, AAV3, AAV4, AAV5, AAV6, AAV7, AAV8, AAV9, AAV10, AAV11, AAV12, Rh10, Rh74 (SEQ ID NO: 35, AAV3B, LK03 (SEQ ID NO: 42), AAV-2i8, SEQ ID NO: 110, SEQ ID NO: 36, and/or SEQ ID NO: 37; or a capsid having 100% sequence identity to AAV1, AAV2, AAV3, AAV4, AAV5, AAV6, AAV7, AAV8, AAV9, AAV10, AAV11, AAV12, Rh10, Rh74 (SEQ ID NO: 35), AAV3B, LK03 (SEQ ID NO: 42) AAV-2i8, SEQ ID NO: 110, SEQ ID NO: 36, and/or SEQ ID NO: 37.
32. The AAV vector of any one of 28-31, wherein the ITRs comprise one or more ITRs of any of AAV1, AAV2, AAV3, AAV4, AAV5, AAV6, AAV7, AAV8, AAV9, AAV10, AAV11, AAV12, Rh10, Rh74, AAV3B AAV serotypes, or a combination thereof.
33. An AAV vector comprising the polynucleotide sequence of any one of SEQ ID NOs: 21-34 and 53-56.
34. A pharmaceutical composition comprising a plurality of AAV vectors of any of 28-33 in a biologically compatible carrier or excipient.
35. The pharmaceutical composition of 34, further comprising empty AAV capsids.
36. The pharmaceutical composition of 35, wherein the ratio of empty AAV capsids to the AAV vector is from about 100:1 to about 50:1, from about 50:1 to about 25:1, from about 25:1 to about 10:1, from about 10:1 to about 1:1, from about 1:1 to about 1:10, from about 1:10 to about 1:25, from about 1:25 to about 1:50, or from about 1:50 to about 1:100.
37. The pharmaceutical composition of any one of 34-36, further comprising a surfactant.
38. A method of treating a subject in need of α-galactosidase A (GLA), comprising administering to the subject a therapeutically effective amount of the polynucleotide or expression cassette of any one of 1-24, or the AAV vector of any one of 25-30, or the pharmaceutical composition of any one of 31-34, wherein the GLA is expressed in the subject.
39. The method of 38, wherein the subject has Fabry disease.
40. The method of 38 or 39, wherein the polynucleotide, expression cassette, AAV vector, or pharmaceutical composition is administered to the subject intravenously, intraarterially, intra-cavity, intramucosally, or via catheter.
41. The method of any one of 38-40, wherein the AAV vector is administered to the subject in a range from about 1×10⁸ to about 1×10¹⁴ vector genomes per kilogram (vg/kg) of the weight of the subject.
42. The method of any one of 38-41, wherein the method reduces, decreases or inhibits one or more symptoms of the need for GLA or of Fabry disease; or prevents or reduces progression or worsening of one or more symptoms of the need for GLA or of Fabry disease; or stabilizes one or more symptoms of the need for GLA or of Fabry disease; or improves one or more symptoms of the need for GLA or of Fabry disease.
43. A cell comprising the polynucleotide or expression cassette of any one of 1-27.
44. A cell that produces the AAV vector of any one of 28-33.
45. A method of producing the AAV vector of any one of 28-33, comprising (a) introducing an AAV vector genome comprising the polynucleotide or expression cassette of any one of 1-22 into a packaging helper cell; and (b) culturing the helper cell under conditions to produce the AAV vector.

A second set of additional aspects and embodiments include:

Aspect 1 directed to a polynucleotide comprising a nucleic acid sequence selected from the group consisting of:
(a) a nucleic acid sequence encoding an α-galactosidase A (GLA), wherein the nucleic acid sequence has a sequence identity of at least about 85% to the sequence of SEQ ID NO: 15, and wherein the GLA has a sequence identity of least 95% to the sequence of SEQ ID NO: 100;
(b) a nucleic acid sequence encoding an α-galactosidase A (GLA) comprising an inserted intron, wherein GLA comprising the inserted intron has a sequence identity of least 95% to the sequence of SEQ ID NO: 100 in the absence of the inserted intron;
(c) a nucleic acid sequence encoding a precursor α-galactosidase A comprising a signal peptide joined to the amino terminus of α-galactosidase A (GLA), wherein the signal peptide has a sequence identity of at least 80% to a sequence selected from the consisting of SEQ ID NO: 41, SEQ ID NO: 57, SEQ ID NO: 58, SEQ ID NO: 60, SEQ ID NO: 61, SEQ ID NO: 62, and SEQ ID NO: 63; and GLA has a sequence identity of least 95% to the sequence of SEQ ID NO: 100;
(d) a nucleic acid encoding an α-galactosidase A (GLA) having an amino acid sequence differing from SEQ ID NO: 100 by 1 to 7 amino acids, wherein at least one of the 1 to 7 amino acids is a substitution selected from the group consisting of Gln57Lys, Gln111Glu, Lys213Glu, Lys237Gln, Phe248Thr, Gly334Glu, and Gly346Asn.

Reference to a “precursor” α-galactosidase A indicates the presence of a signal peptide. The signal peptide may be the naturally occurring peptide associated with GLA or a different or heterologous signal peptide.

In a different embodiments of aspect 1(a) the nucleic acid has a sequence identity of at least 90%, at least 95%, at least 98%, or 100% to the sequence of SEQ ID NOs: 15, 16, 17 or 18 or a sequence identity of at least 90%, at least 95%, at least 98%, or 100% to bases 1-1194 of the sequence of SEQ ID NO: 15, 16, 17, or 18 (bases 1-1194 provide the amino acid coding region); and independently GLA has a sequence identify to the sequence of SEQ ID NO: 100 of at least 95%, at least 98% or 100%. Reference to independently indicates any of the provided sequence identities to the sequence of SEQ ID NO: 15 may be combined with any of the provided sequence identities of the sequence of SEQ ID NO: 100. For example, a nucleic acid having a sequence identity to the sequence of SEQ ID NO: 15 of at least 90%, may encode GLA having a sequence identity of least 95%, at least 98%, or 100% to the sequence of SEQ ID NO: 100; a nucleic acid having a sequence identity to the sequence of SEQ ID NO: 15 of at least 95%, may encode GLA having a sequence identity of least 95%, at least 98%, or 100% to the sequence of SEQ ID NO: 100; and a nucleic acid of the sequence of SEQ ID NO: 15 may encode GLA having a sequence identity of least 95%, at least 98%, or 100% to the sequence of SEQ ID NO: 100.

In different embodiments of aspect 1(b) the sequence identity of GLA to the sequence of SEQ ID NO: 100 in the absence of the inserted intron is at least 98% or 100%.

In different embodiments of aspect 1(c) the signal peptide has a sequence identity to the sequence of SEQ ID NO: 41 of at least 85%, at least 90%, at least 95% or 100%; has a sequence identity to the sequence of SEQ ID NO: 57 of at least 85%, at least 90%, at least 95% or 100%; has a sequence identity to the sequence of SEQ ID NO: 58 of at least 85%, at least 90%, at least 95% or 100%; has a sequence identity to the sequence of SEQ ID NO: 59 of at least 85%, at least 90%, at least 95% or 100%; has a sequence identity to the sequence of SEQ ID NO: 60 of at least 85%, at least 90%, at least 95% or 100%; has a sequence identity to the sequence of SEQ ID NO: 61 of at least 85%, at least 90%, at least 95% or 100%; has a sequence identity to the sequence of SEQ ID NO: 62 of at least 85%, at least 90%, at least 95% or 100%; or has a sequence identity to the sequence of SEQ ID NO: 63 of at least 85%, at least 90%, at least 95% or 100%; each independently with respect to GLA having a sequence identity of least 95%, at least 98%, or 100% to the sequence of SEQ ID NO: 100. Reference to each independently indicates that each of the provided signal sequences with accompanying sequence identify can be combined with GLA having any of the provided sequence identities to the sequence of SEQ ID NO: 100. For example, a signal peptide having a sequence identity to the sequence of SEQ ID NO: 41 of at least 85% may be combined with GLA having a sequence identity of least 95%, at least 98%, or 100% to the sequence of SEQ ID NO: 100; a signal peptide having a sequence identity to the sequence of SEQ ID NO: 41 of at least 90%, may be combined with a GLA having a sequence identity of least 95%, at least 98%, or 100% to the sequence of SEQ ID NO: 100; a signal peptide having a sequence identity to the sequence of SEQ ID NO: 41 of at least 95%, may be combined with GLA having a sequence identity of least 95%, at least 98%, or 100% to the sequence of SEQ ID NO: 100; and a signal peptide of the sequence of SEQ ID NO: 41, may be combined with a GLA having a sequence identity of least 95%, at least 98%, or 100% to the sequence of SEQ ID NO: 100.

In a further embodiments of aspect 1(c) the precursor α-galactosidase A comprising a signal peptide has an amino sequence at least 95%, 97%, 99%, or 100% identity to a sequence selected from the group consisting of SEQ ID NOs: 101, 102, 103, 104, 105, 106, 107, 108 and 109; and the precursor α-galactosidase A comprising a signal peptide has an amino sequence at least 95%, 97%, 99%, or 100% to SEQ ID NO: 109.

In different embodiments of aspect 1(d) the nucleic acid encoding GLA differs from the sequence of SEQ ID NO: 100 by 1 to 7 amino acids substitutions wherein each of the 1, 2, 3, 4, 5, 6, or 7 amino acid substitutions are selected from the group consisting of Gln57Lys, Gln111Glu, Lys213Glu, Lys237Gln, Phe248Thr, Gly334Glu, and Gly346Asn; and the GLA comprises the amino acid sequence of SEQ ID NO: 48.

Embodiment 1 further describes aspect 1(d) and different embodiments of aspect 1(d) by providing the nucleic acid comprises a sequence identity of at least 90%, at least 95%, at least 98%, to SEQ ID NO: 47 or bases 1-1194 of SEQ ID NO 47, or is provided by the sequence of SEQ ID NO: 47 or bases 1-1194 of SEQ ID NO 47.

Reference to further describing an aspect or embodiment provides that the further description applies to each of the descriptions provided in the reference aspect or embodiment. For example, embodiment 1 further describing aspect 1(d) and different embodiments of aspect 1(d), provides that the different embodiments in embodiment 1 can apply independently to any descriptions provided in the aspect 1(d) and different embodiments of aspect 1(d).

Embodiment 2 further describes aspect 1(b) by providing the GLA provided in the absence of the intron comprises the GLA sequence as provided in any of aspect 1(a) and different embodiments of aspect 1(a), aspect (1b) and different embodiments of aspect 1(b), aspect 1(d) and different embodiment of aspect 1(d), or embodiment 1, wherein the intron is positioned between nucleotides 78 and 79 of GLA, wherein the nucleotide positions are given in reference to the coding sequence of GLA of SEQ ID NO: 14.

Embodiment 3 further describes aspect 1(d) and different embodiments of aspect 1(d), and embodiment 2 by providing the intron has a sequence identity of at least 90%, at least 95%, at least 98%, or 100% to the sequence of SEQ ID NOs: 49, 50, 51, or 52.

Embodiment 4 further describes aspect 1(d) by providing GLA comprising the intron has a sequence identity of at least 90%, at least 95%, at least 98%, or 100% to the sequence of SEQ ID NOs: 43, 44, 45 or 46.

Embodiment 5 further describes aspect 1(a) and different embodiments of aspect 1(a), aspect 1(b) and different embodiments of aspect 1(b), aspect 1(d) and different embodiment of aspect 1(d), and embodiments 1, 2, 3, and 4 by providing the polynucleotide further comprises a second nucleic acid sequence, wherein the second sequence encodes a signal peptide sequence positioned at the 5′ end of the GLA nucleic acid sequence. The signal peptide sequence may be a heterologous, endogenous or native signal peptide sequence, or a derivative thereof.

Embodiment 6, further describes embodiment 5 by providing in different embodiments, the peptide signal has a sequence identity to the sequence of SEQ ID NO: 41 of at least 85%, at least 90%, at least 95% or 100%; has a sequence identity to the sequence of SEQ ID NO: 57 of at least 85%, at least 90%, at least 95% or 100%; has a sequence identity to the sequence of SEQ ID NO: 58 of at least 85%, at least 90%, at least 95% or 100%; has a sequence identity to the sequence of SEQ ID NO: 59 of at least 85%, at least 90%, at least 95% or 100%; has a sequence identity to the sequence of SEQ ID NO: 60 of at least 85%, at least 90%, at least 95% or 100%; has a sequence identity to the sequence of SEQ ID NO: 61 of at least 85%, at least 90%, at least 95% or 100%; has a sequence identity to the sequence of SEQ ID NO: 62 of at least 85%, at least 90%, at least 95% or 100%; or has a sequence identity to the sequence of SEQ ID NO: 63 of at least 85%, at least 90%, at least 95% or 100%.

Embodiment 7 further describes the GLA encoding sequence as provided in any of aspect 1(a) and different embodiments of aspect 1(a), aspect (1b) and different embodiments of aspect 1(b), aspect (1c) and different embodiments of aspect 1(c), aspect 1(d) and different embodiment of aspect 1(d), and embodiments 1-6, wherein the GLA encoding sequence contains fewer than 14, 13, 12, 11, 10, 9, 8, 7, 6, 5, 4, 3, 2, or 1 CpG dinucleotides.

Aspect 2 is directed to an expression cassette comprising the polynucleotide provided in aspect 1(a) and different embodiments of aspect 1(a), aspect (1b) and different embodiments of aspect 1(b), aspect (1c) and different embodiments of aspect 1(c), aspect 1(d) and different embodiment of aspect 1(d), and embodiments 1-7; wherein the polynucleotide is operatively coupled to an expression control element.

Embodiment 8 further describes aspect 2, wherein the polypeptide description is as provided in the aspect 2 and in different embodiments of aspect 2, the expression control element is a liver-specific expression control element, the expression control element comprises an ApoE/hAAT enhancer/promoter sequence, or comprises the sequence of SEQ ID NO: 38 or a sequence with a sequence identity of at least 98% to SEQ ID NO: 38.

Reference to a description in a referred to aspect or embodiment provides for incorporation of the referred aspect (including associated embodiments) and referred to embodiments (including different described embodiments). For example, reference to the polypeptide description provided in aspect 2, describes in different embodiments the polynucleotide provided in aspect 1(a) and different embodiments of aspect 1(a); aspect (1b) and different embodiments of aspect 1(b), aspect 1(c) and different embodiments of aspect 1(c), aspect 1(d) and different embodiment of aspect 1(d), and embodiments 1-7.

Embodiment 9 further describes the expression control element of embodiment 8 by providing the expression control is positioned 5′ of the polynucleotide.

Embodiment 10 further describes the expression cassette of aspect 2, embodiment 8 and embodiment 9, wherein the expression cassette further comprises a poly-adenylation sequence positioned 3′ of the polynucleotide. In further embodiments the poly-adenylation sequence comprises a bovine growth hormone (bGH) polyadenylation sequence; and comprises a sequence with a sequence identity to SEQ ID NO: 20 of at least 95% or 100%.

Embodiment 11 further describes the expression cassette of aspect 2 and embodiments 9-10, wherein an intron is positioned between the 3′ end of the expression control element and the 5′ end of the polynucleotide. In further embodiments the intron comprises a sequence with a sequence identity to the sequence of SEQ ID NO: 39 of at least 95% or 100%.

Embodiment 12 further describes the expression cassette of aspect 2, and embodiments 9-11, wherein the expression control element and/or the poly-adenylation sequence is CpG-reduced compared to the wild-type expression control element or polyadenylation sequence.

Embodiment 13 further describes the polypeptide of aspect 1 (including 1(a), 1(b), 1(c) and 1(d) and related embodiments) and embodiments 1-8, and the expression vector of aspect 2 and embodiments 9-12, wherein the polypeptide or expression cassette further comprises an AAV inverted repeat (ITR) flanking its 5′ terminus and/or an AAV ITR flanking its 3′ terminus. Preferably, the 3′ and 5′ terminus are flanked by an ITR.

Aspect 3 is directed to an AAV plasmid genome comprising the polynucleotide or expression cassette of embodiment 13 and an origin of replication is present. In a further embodiment, a selectable marker is present.

Aspect 4 is directed to an adeno-associated virus (AAV) vector comprising a capsid and the polynucleotide or the expression cassette provided in embodiment 13. Reference to AAV capsid includes naturally occurring AAV capsids along with modified and variant AAV capsids. The AAV capsid facilitates intracellular delivery of the polynucleotide or expression cassette, preferably the expression cassette.

Embodiment 14 further describes the AAV vector of aspect 4, and the polypeptide and expression cassettes of embodiment 13, wherein the ITRs comprise one or more ITRs of any of AAV1, AAV2, AAV3, AAV4, AAV5, AAV6, AAV7, AAV8, AAV9, AAV10, AAV11, AAV12, Rh10, Rh74, or AAV3B serotypes, or a combination thereof.

Embodiment 15 further describes aspect 4, embodiment 13 and embodiment 14, wherein in different embodiments: adjacent to the 5′ ITR at the 5′ end is a 5′ cloning remnant and/or adjacent to the 3′ ITR at the 3′ end is a 3′ cloning remnant.

Embodiment 16 further describes aspect 4 and embodiments 13-15, wherein the 5′ and/or 3′ ITR is modified to have reduced CpGs.

Embodiment 17 further describes the AAV vector of aspect 4 and embodiments 13-16, wherein the expression cassette comprises a sequence having a sequence identity of at least 95%, at least 98%, or 100% to the sequence of any one of SEQ ID NOs: 21-34, 53-56 and 91-99.

Embodiment 18 further describes the AAV vector of aspect 4 and embodiments 13-17, wherein the expression cassette comprises or consists of a sequence having a sequence identity of at least 95%, at least 98% or 100% with a sequence of any one of SEQ ID NOs: 91-95.

Embodiment 19 further describes the capsid of aspect 4 and embodiments 13-18, wherein the capsid comprises VP1, VP2 and/or VP3 protein having a sequence identity of at least 90%, at least 95%, at least 98%, or 100% to VP1, VP2 and/or VP3 provided by AAV1, AAV2, AAV3, AAV4, AAV5, AAV6, AAV7, AAV8, AAV9, AAV10, AAV11, AAV12, Rh10, Rh74 (SEQ ID NO: 35), AAV3B, LK03 (SEQ ID NO: 42) or AAV-2i8; comprises VP1 having a sequence identity of at least 90%, at least 95%, at least 98%, or 100% to the sequence of SEQ ID NO: 110 or 42; or comprises VP1 having a sequence identity of at least 90%, at least 95%, at least 98%, or 100% to the sequence of SEQ ID NOs: 110 or 42, and/or VP2 having a sequence identity of at least 90%, at least 95%, at least 98%, or 100% to the sequence of SEQ ID NO: 36 and/or VP3 having a sequence identity at least 90%, at least 95%, at least 98%, or 100% to the sequence of SEQ ID NO: 37.

Aspect 5 is directed to a pharmaceutical composition comprising the AAV vector provided for in aspect 4 and embodiments 13-19 and a biologically compatible carrier or excipient.

Embodiment 20, further describes the pharmaceutical composition of aspect 5, wherein the AAV vector is provided in an effective amount to increase GLA activity in a human subject, and preferably decrease globotriaosylsphingosine.

Embodiment 21 further describes the pharmaceutical composition of aspect 5, wherein the composition further comprises empty AAV capsids. Reference to empty AAV capsids, indicates the same capsids as used in a AAV vector being administered, but the capsid lacks the AAV vector. In further embodiments the ratio of empty AAV capsid to the AAV vector is from about 100:1 to 1:100; from about 100:1 to about 50:1; from about 50:1 to about 25:1; from about 25:1 to about 10:1; from about 10:1 to about 1:1; from about 1:1 to about 1:10; from about 1:10 to about 1:25; from about 1:25 to about 1:50; or from about 1:50 to about 1:100.

Embodiment 22 further describes the pharmaceutical composition of aspect 5 and embodiments 20 and 21, wherein the composition further comprises a surfactant.

Aspect 6 is directed to a polypeptide selected from the group consisting of: (a) a nucleic acid sequence encoding a precursor α-galactosidase A comprising a signal peptide joined to the amino terminus of α-galactosidase A, wherein the signal peptide has a sequence identity of at least 80% to a sequence selected from the consisting of SEQ ID NO: 41, SEQ ID NO: 57, SEQ ID NO: 58, SEQ ID NO: 59, SEQ ID NO: 60, SEQ ID NO: 61, SEQ ID NO: 62, and SEQ ID NO: 63; and said GLA has a sequence identity of least 95% to the sequence of SEQ ID NO: 100; and
(b) an α-galactosidase A (GLA) having an amino acid sequence differing from the sequence of SEQ ID NO: 100 by 1 to 7 amino acids, wherein at least one of said 1 to 7 amino acids is a substitution selected from the group consisting of Gln57Lys, Gln111Glu, Lys213Glu, Lys237Gln, Phe248Thr, Gly334Glu, and Gly346Asn.

In different embodiments of aspect 6(a), the signal peptide has a sequence identity to the sequence of SEQ ID NO: 41 of at least 85%, at least 90%, at least 95% or 100%; has a sequence identity to the sequence of SEQ ID NO: 57 of at least 85%, at least 90%, at least 95% or 100%; has a sequence identity to the sequence of SEQ ID NO: 58 of at least 85%, at least 90%, at least 95% or 100%; has a sequence identity to the sequence of SEQ ID NO: 60 of at least 85%, at least 90%, at least 95% or 100%; has a sequence identity to the sequence of SEQ ID NO: 61 of at least 85%, at least 90%, at least 95% or 100%; has a sequence identity to the sequence of SEQ ID NO: 62 of at least 85%, at least 90%, at least 95% or 100%; or has a sequence identity to the sequence of SEQ ID NO: 63 of at least 85%, at least 90%, at least 95% or 100%; each independently with respect to a GLA having a sequence identity of least 95%, at least 98%, or 100% to the sequence of SEQ ID NO: 100.

In different embodiments of aspect 6(b), GLA has an amino acid sequence differing from SEQ ID NO: 100 by 1 to 7 amino acids substitutions, wherein the 1, 2, 3, 4, 5, 6, or 7 amino acid substitutions are each independently selected from the group consisting of Gln57Lys, Gln111Glu, Lys213Glu, Lys237Gln, Phe248Thr, Gly334Glu, and Gly346Asn; and the GLA comprises the amino acid sequence of SEQ ID NO: 48.

Aspect 7 is directed to a method of treating a subject in need of GLA comprising administering to the subject a therapeutically effective amount of the polynucleotide, expression cassette, AAV vector, pharmaceutical composition, or polypeptide or any of aspects and embodiments described above in the second set of additional aspects and embodiments. Preferably, the subject is a human.

Embodiment 23 further describes aspect 7, wherein the subject has Fabry disease; the method reduces, decreases or inhibits one or more symptoms of the need for GLA or of Fabry disease; the method prevents or reduces progression or worsening of one or more symptoms of the need for GLA or of Fabry disease; the method stabilizes one or more symptoms of the need for GLA or of Fabry disease; or the method improves one or more symptoms of the need for GLA or of Fabry disease.

Embodiment 24 further describes aspect 7 and embodiment 23, wherein the AAV vector is administered to the subject in a range from about 1×10⁸ to about 1×10¹⁴ vector genomes per kilogram (vg/kg) of the weight of the subject.

Aspect 8 is directed the polynucleotide, expression cassette, AAV vector, pharmaceutical composition, or polypeptide or any of aspects and embodiments described above in the second set of additional aspects and embodiments for use in (i) a method described in aspect 7, embodiment 22 and embodiment 23; or (ii) preparation of medicament.

All of the features disclosed herein can be combined in any combination. Each feature disclosed in the specification can be replaced by an alternative feature serving a same, equivalent, or similar purpose. Thus, unless expressly stated otherwise, disclosed features (e.g., modified nucleic acids encoding GLA, expression cassettes comprising modified nucleic acids encoding GLA, rAAV particles comprising the modified nucleic acids encoding GLA, and non-viral vectors comprising the modified nucleic acids encoding GLA) are an example of a genus of equivalent or similar features.

A number of embodiments of the instant invention have been described. Nevertheless, one skilled in the art, without departing from the spirit and scope of the instant invention, can make various changes and modifications of the instant invention to adapt it to various usages and conditions. Accordingly, the following examples are intended to illustrate, but not limit the scope of the instant invention claimed in any way.

### EXAMPLES

### Example 1—GLA Expression Cassette Overview

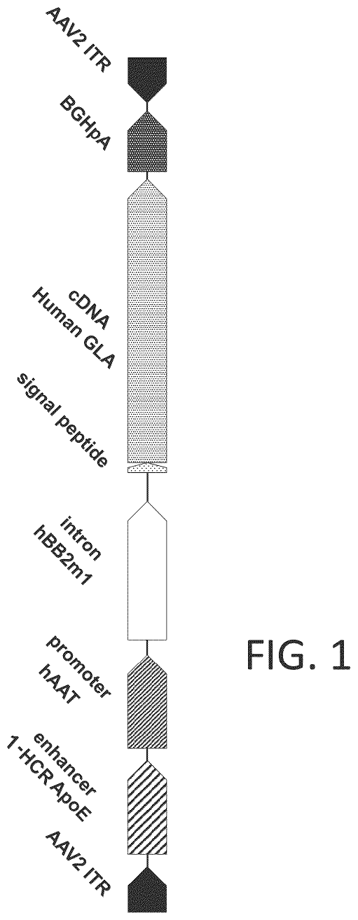

GLA expression cassettes were designed as shown in FIG. 1 and in Table 2. Table 2 references the sequences for the precursor α-galactosidase A (signal peptide+GLA moiety) and the expression cassettes. All sequences provided in Table 2 contained 5′ and 3′ flanking AAV inverted terminal repeats (ITRs), a liver-specific ApoE/hAAT enhancer/promoter sequence, a human hemoglobin subunit beta (HBB2) intron, a signal peptide, a human GLA coding sequence, and a bovine growth hormone (bGH) polyadenylation (poly A) sequence.

TABLE 2
GLA expression cassette components

| Expression Cassette | | Signal Peptide | | GLA Encoding Moiety | |
|---|---|---|---|---|---|
| | SEQ | | SEQ | | SEQ |
| | ID | | ID | | ID |
| Name | NO | Name | NO | Name | NO |
| pAAV_ApoE_hAAT_HBB2 | 21 | sp7 | 1 | GLA | 14 |
| m1_sp7.GLA_BGH228 | | | | | |
| pAAV_ApoE_hAAT_HBB2 | 22 | spAHSG | 6 | GLA | 14 |
| m1_spAHSG.GLA_BGH228 | | | | | |
| pAAV_ApoE_hAAT_HBB2 | 23 | spCD300 | 7 | GLA | 14 |
| m1_spCD300.GLA_BGH228 | | | | | |
| pAAV_ApoE_hAAT_HBB2 | 24 | spGLA | 8 | GLA | 14 |
| m1_spGLA.GLA_BGH228 | | | | | |
| pAAV_ApoE_hAAT_HBB2 | 25 | spLAMP1 | 9 | GLA | 14 |
| m1_spLAMP1.GLA_BGH228 | | | | | |
| pAAV_ApoE_hAAT_HBB2 | 26 | spNotch2 | 10 | GLA | 14 |
| m1_spNotch2.GLA_BGH228 | | | | | |
| pAAV_ApoE_hAAT_HBB2 | 27 | spORM1 | 11 | GLA | 14 |
| m1_spORM1.GLA_BGH228 | | | | | |
| pAAV_ApoE_hAAT_HBB2 | 29 | spTF | 13 | GLA | 14 |
| m1_spTF.GLA_BGH228 | | | | | |
| pAAV_ApoE_hAAT_HBB2 | 91 | sp7 | 2 | GLAco4 | 15 |
| m1_sp7.GLAco4_BGH228 | | | | | |
| pAAV_ApoE_hAAT_HBB2 | 92 | sp7 | 3 | GLAcoBC0 | 16 |
| m1_sp7.GLAcoBC0_BGH228 | | | | | |
| pAAV_ApoE_hAAT_HBB2 | 93 | sp7 | 4 | GLAcoH0 | 17 |
| m1_sp7.GLAcoH0_BGH228 | | | | | |
| pAAV_ApoE_hAAT_HBB2 | 94 | sp7 | 5 | GLAcoH6 | 18 |
| m1_sp7.GLAcoH6_BGH228 | | | | | |
| pAAV_ApoE_hAAT_HBB2 | 95 | sp7 | 2 | GLAv45 | 19 |
| m1_sp7.GLAv45_BGH228 | | | | | |
| pAAV_ApoE_HAT_sp7_GLA_WT_intronIgHA | 96 | sp7 | 2 | GLA_WT_intronIgHA | 45 |
| pAAV_ApoE_HAT_sp7_GLA_WT_intronIgHμ | 97 | sp7 | 2 | GLA_WT_intronIgHμ | 46 |
| pAAV_ApoE_HAT_sp7_GLA_WT_intronRBP4 | 98 | sp7 | 2 | GLA_WT_intronRBP4 | 44 |
| pAAV_ApoE_HAT_sp7_GLA_WT_intronVTN1 | 99 | sp7 | 2 | GLA_WT_intronVTN1 | 43 |
| pSPKL0031 (GLA 7 mut) | 28 | sp7 | 2 | GLA 7 mut | 47 |

Expression cassettes are packaged in an AAV viral particle by being encapsidated in an AAV capsid, e.g., AAV-4-1 capsid variant, described in International Patent Application publication WO 2016/210170, the contents of which are incorporated herein in their entirety, or LK03 capsid variant, described in U.S. Pat. No. 9,169,299, the contents of which are incorporated herein in their entirety. Viral particles are generally produced using the triple transfection protocol well-known in the art.

### Example 2—Evaluation of Signal Peptides

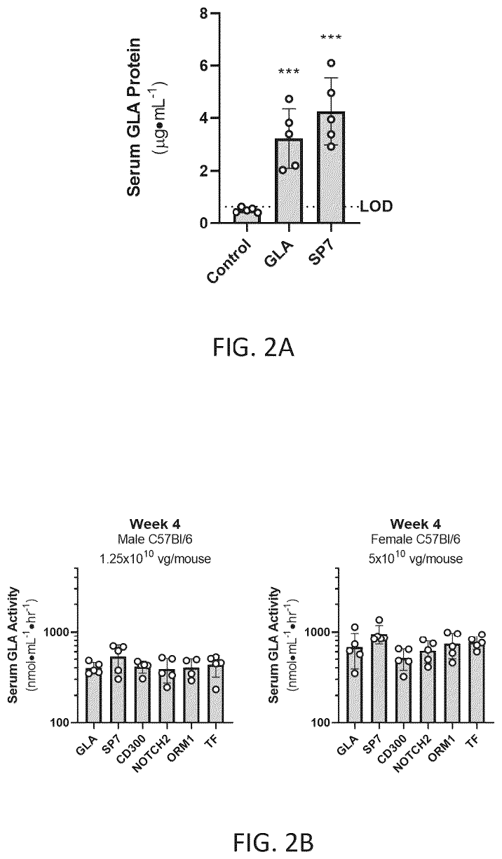

To evaluate the potency of GLA expression cassettes having different signal peptides in place of the native GLA signal peptide, expression cassettes having the sequences of SEQ ID NO: 21 (sp7.GLA), SEQ ID NO: 23 (spCD300.GLA), SEQ ID NO: 24 (spGLA.GLA), SEQ ID NO: 26 (spNotch2.GLA), SEQ ID NO: 27 (spORM1.GLA), and SEQ ID NO: 29 (spTF.GLA) were packaged into AAV vectors comprising SEQ ID NOs: 110, 36 and 37 capsids. Five to six male or female C57Bl/6 mice per group were injected intravenously via the tail vein with 1.25×10¹⁰ vg/mouse or 5×10¹⁰ vg/mouse of the rAAVs, respectively. Levels of circulating GLA enzyme activity in mouse serum were measured using an in vitro enzyme activity assay. A standard curve was generated from serial dilutions of fluorescent 4-methylumbelliferyl (4-MU). GLA enzyme activity was defined as concentration of fluorescent 4-MU released per hour of co-incubation of serum and synthetic enzyme substrate 4-methylumbelliferyl β-D-galactopyranoside (4-MU-Gal), in units of nmol×mL⁻¹×hr⁻¹. Incubations of individual tissue samples containing GLA and 4-MU-Gal were performed in duplicate for all measurements of GLA enzyme activity. As shown in FIG. 2A, the expression cassette containing signal peptide sp7, which is chymotrypsin B2-derived, yielded higher levels of serum GLA activity compared to the expression cassette containing the wild-type GLA signal peptide.

To evaluate the human GLA protein expression levels of GLA cassettes over endogenous levels of mouse GLA in serum, expression cassettes having the sequence of SEQ ID NO: 21 (sp7.GLA) and the sequence of SEQ ID NO: 24 (spGLA.GLA) were each packaged into an AAV vector comprising SEQ ID NOs: 110, 36 and 37 capsids. Five female C57Bl/6 mice per group were injected intravenously via the tail vein with 5×10¹⁰ vg/mouse of rAAV. Serum GLA protein was measured using capillary electrophoresis. Levels of circulating GLA in mouse sera collected six weeks post-transduction were quantified against a standard curve of recombinant human GLA (ProteinSimple Wesm, Bio-Techne) and plotted in units of μg/mL (FIG. 2B). GLA protein levels were also determined in untreated control C57Bl/6 mice. Bar heights represent mean serum GLA activity of five mice per group; error bars indicate one standard deviation from the mean; the quantification limit of the standard curve is shown by a horizontal line (lower limit of quantitation, indicated as “LOD” in the figure). Levels of GLA protein were significantly higher in mice treated with either rAAV (AAV-sp7.GLA or AAV-spGLA.GLA) than in untreated mice (t-test, ***p<0.001). Both the rAAV vector expressing GLA with its native signal peptide (GLA) and the rAAV vector expressing GLA with the chymotrypsinogen B2 signal peptide (SP7) induced serum GLA expression over baseline endogenous levels of mouse GLA in C57Bl/6 mice (FIG. 2B).

### Example 3—Evaluation of GLA Cassettes in Fabry Mouse Models

The male GLA-/null knockout mouse model was used to assess efficacy of expression cassettes described herein. This mouse model has a mixed B6; 129 background, rather than a C57Bl/6 background.

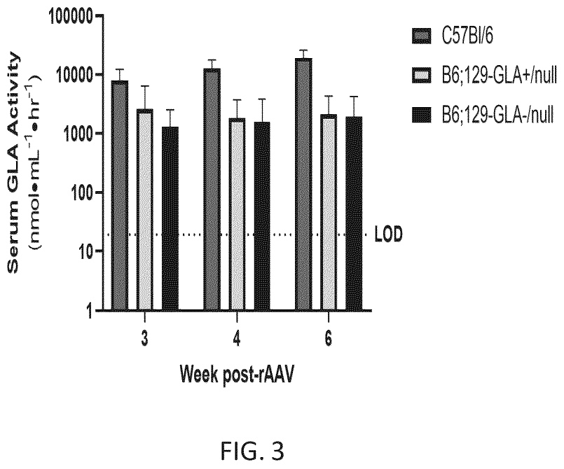

Ten C57Bl/6, eleven B6; 129-GLA+/null and eleven B6; 129-GLA-/null male mice were injected intravenously via the tail vein with 5×10¹⁰ vg/mouse of the rAAV (SEQ ID NOs: 110, 36 and 37 capsids) comprising the expression cassette having the sequence of SEQ ID NO: 21 (sp7.GLA) (referred to herein as AAV-sp7-GLA). Activity of GLA in mouse serum collected three, four, and six weeks following rAAV administration was defined as concentration of fluorescent 4-MU released per hour of co-incubation of serum and synthetic substrate 4-MU-Gal, in units of nmol×mL⁻¹×hr⁻¹ and is plotted in FIG. 3. Bar heights indicate the mean of ten to eleven mice per group; error bars indicate one standard deviation from the mean; the quantification limit of the standard curve 4-parameter fit is shown by a horizontal line (lower limit of quantitation, indicated as “LOD” in the figure). AAV-sp7-GLA rAAV exhibited greater potency in C57Bl/6 mice compared to B6; 129 mice. Serum GLA activity post-transduction was low in both B6; 129-GLA+/null and B6; 129-/null mice, thus lower potency was not a specific consequence of the GLA genotype or the Fabry phenotype. Consequently, in order to achieve serum GLA activities in excess of 1000 nmol/mL-hr in Fabry model mice (B6; 129 GLA null strain), dose escalation studies were performed to explore GLA expression at a range of AAV doses from 5×10⁹ up to 5×10¹¹ vg/mouse.

### Example 4—Dose Ranging Studies

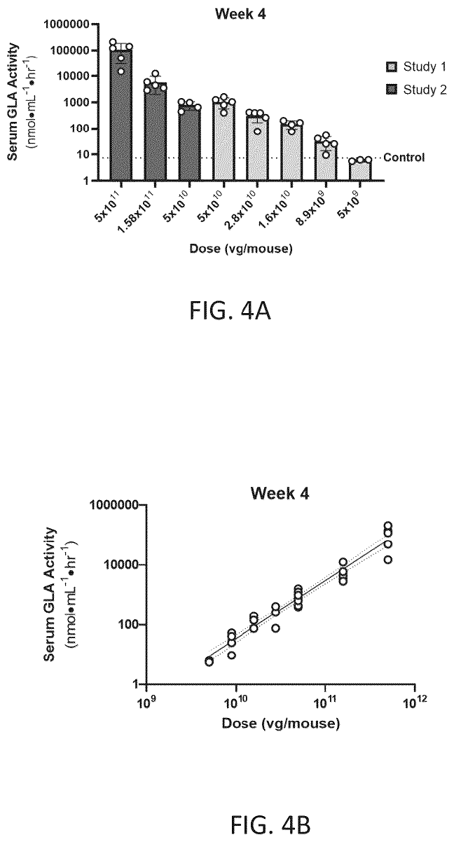

B6; 129 GLA-/null male mice (n=5 per group) were injected intravenously via the tail vein with 5×10⁹ vg/mouse, 8.9×10⁹ vg/mouse, 1.6×10¹⁰ vg/mouse, 2.8×10¹⁰ vg/mouse, 5×10¹⁰ vg/mouse, 1.58×10¹¹ vg/mouse, or 5×10¹¹ vg/mouse of AAV-sp7-GLA. Dose escalation was performed in two separate studies, Study 1 and Study 2. Activity of GLA in mouse serum collected 4 weeks post transduction was measured as concentration of fluorescent 4-MU released per hour of co-incubation of serum and synthetic substrate 4-MU-Gal, in units of nmol×mL⁻¹×hr⁻¹, and is plotted in FIG. 4A and FIG. 4B. Bar heights represent mean serum GLA activity of five mice per group; error bars indicate one standard deviation from the mean. The maximum level of background serum GLA activity detected by the assay in five un-transduced control GLA-/null mice at week 4 is shown as a horizontal line in FIG. 4A. As shown, GLA expression showed a linear dose-response to the amount of AAV administered (FIG. 4B).

### Example 5—Kidney Uptake of GLA

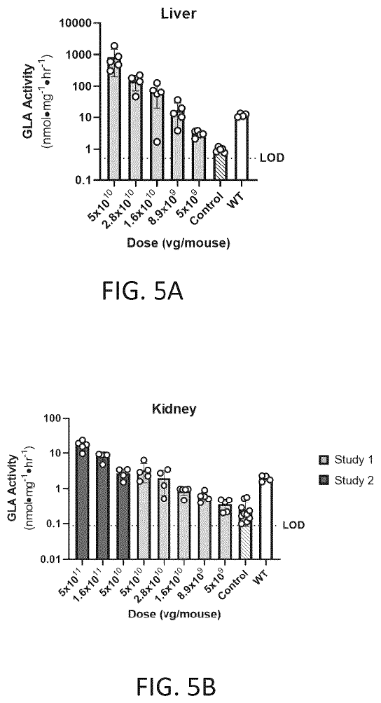

Tissue uptake of GLA, especially in kidney, is thought to be essential for clinical efficacy of Fabry therapeutics. Groups of five B6; 129 GLA-/null male mice were injected intravenously via the tail vein with 5×10⁹ vg/mouse, 8.9×10⁹ vg/mouse, 1.6×10¹⁰ vg/mouse, 2.8×10¹⁰ vg/mouse, or 5×10¹⁰ vg/mouse of AAV-sp7-GLA. Livers and kidneys were collected twelve weeks post-transduction, and GLA activity in tissue lysates was measured as concentration of fluorescent 4-MU released per hour of co-incubation of tissue lysate and synthetic substrate 4-MU-Gal, in units of nmol×mg total lysate protein⁻¹×hr⁻¹, and is plotted in FIG. 5A (liver) and FIG. 5B (kidney). GLA activity in liver and kidney tissue lysates was also assessed in five untreated B6; 129 GLA-/null male control mice, and in four untreated age-matched B6; 129 GLA+/null (WT) male mice. Bar heights represent mean tissue GLA activity; error bars indicate one standard deviation from the mean; a horizontal line indicates the quantitative limit of the standard curve normalized to protein concentrations of respective tissue lysates. As shown, dose escalation of AAV resulted in increased GLA activity observed in the liver and in the kidney of knockout male mice. Moreover, at doses equal to or greater than 2.8×10¹⁰ vg/mouse, GLA activity in the livers and kidneys of GLA knockout animals was restored to the levels observed in normal animals.

Notably, the 5×10¹⁰ vg/mouse dose of the AAV-sp7-GLA vector, which contains a liver-specific transgene promoter, achieved GLA activity in the kidneys of Fabry mice approaching the levels of GLA enzymatic activity associated with wild-type mice (B6; 129 GLA+/null, WT).

### Example 6—Evaluation of GLA Coding Sequences

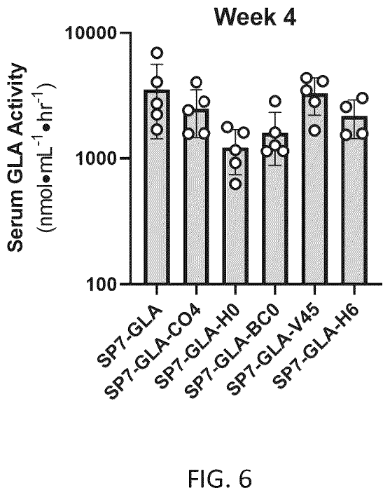

The GLA portion of the expression cassette having the sequence of SEQ ID NO: 21 (sp7.GLA) was depleted of CpG motifs and was codon-optimized to support maximal expression. Codon-optimized GLA variant cassettes (SEQ ID NO: 91 (GLAco4), SEQ ID NO: 92 (GLAcoBCO), SEQ ID NO: 93 (GLAcoHO), SEQ ID NO: 94 (GLAcoH6), and SEQ ID NO: 95 (GLAv45)) were encapsidated in an AAV capsid and transduced into five male C57Bl/6 mice per group at a dose of 5.0×10¹⁰ vg/mouse. Serum GLA activity at week 4 was measured as a proxy for relative transgene activity, and plotted in FIG. 6. Bar heights represent mean tissue GLA activity; error bars indicate one standard deviation from the mean. As shown, CpG-free or CpG-reduced, codon-optimized variants demonstrated serum GLA activity comparable to that of the cassette having the sequence of SEQ ID NO: 21.

### Example 7—Further Optimization of GLA Expression Cassette

Additional modifications were made to the GLA portion of the expression cassette provided in the sequence of SEQ ID NO: 21 (sp7.GLA). A GLA variant containing 7 amino acid substitutions (Q57K; Q111E; K213E; K237Q; F248T; G334E; G346N) (“GLA 7 mut”) was generated using structurally guided mutagenesis (SEQ ID NO: 47 (SPKL0031)).

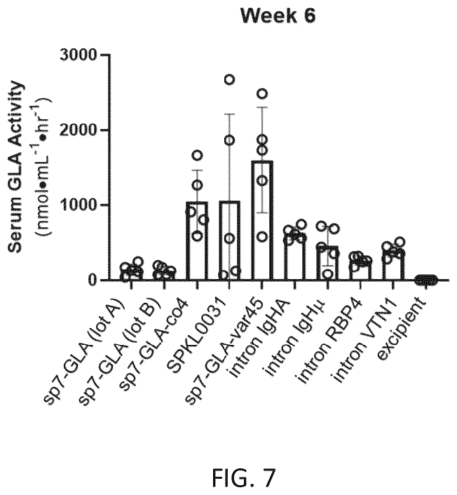

Additionally, heterologous introns were introduced into the sp7-GLA coding sequence between nucleotides 78/79 of the GLA coding sequence SEQ ID NO: 14 to provide SEQ ID NO: 96 (IgHA, SEQ ID NO: 97 (IgHμ), SEQ ID NO: 98 (RBP4), and SEQ ID NO: 99 (VTN1)). Expression cassette SEQ ID NO: 95 (referred to as sp7-GLA-var45 in FIG. 7) was also included. All cassette sequences were encapsidated in an AAV capsid and transduced into five male C57Bl/6 mice per group at a dose of 2.5×10¹⁰ vg/mouse co-administered with 2×10⁹ vg/mouse of AAV-CAG-Gaussia (AAV encapsidated expression cassette having Gaussia luciferase under the control of the CAG promoter, for purposes of normalizing transduction efficiency). Serum GLA activity at week 6 was measured as a proxy for relative transgene activity, and plotted in FIG. 7. Bar heights represent mean tissue GLA activity; error bars indicate one standard deviation from the mean. All modifications except the insertion of the IgH and RBP4 introns led to a statistically significant increase in GLA activity in mouse serum.

### Example 8—Dose Escalation Study with Codon-Optimized AAV-Sp7-GLA in Fabry Disease Model Mice

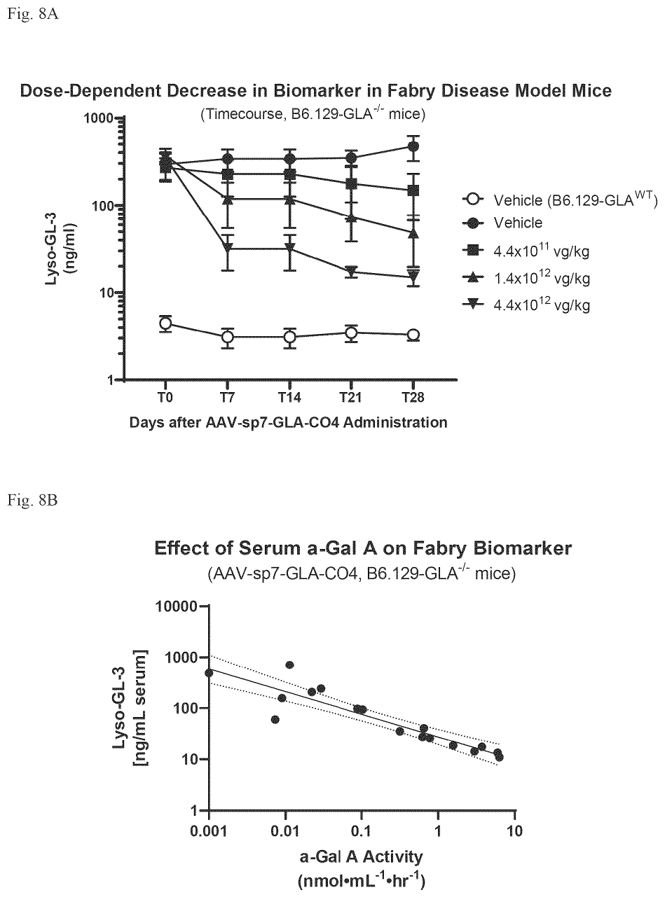

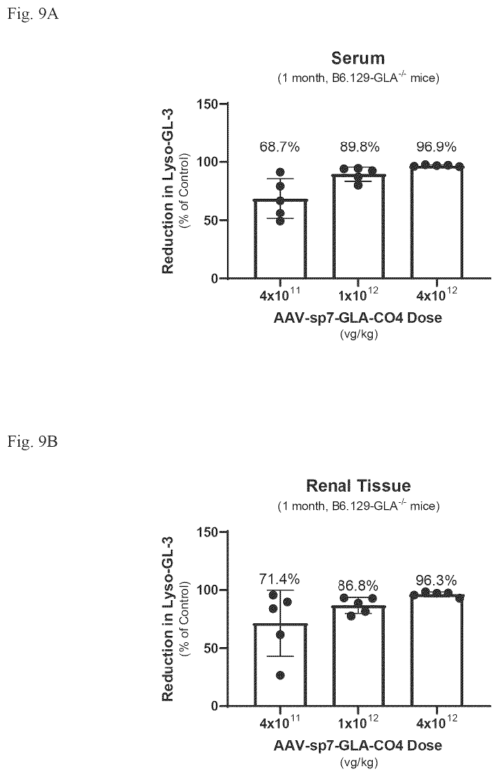

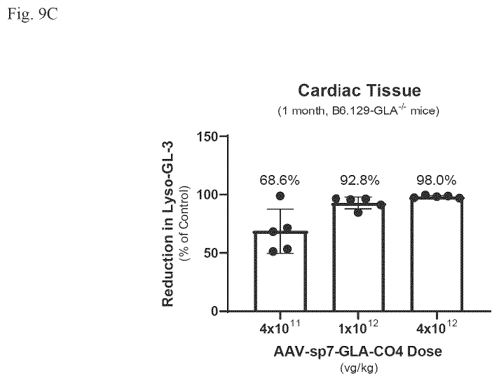

GLA knockout mice (in groups of five male B6; GLA−/−) were intavenously administered, via the tail vein, three doses (4.4E11, 1.4E12 and 4.4E12 vg/kg) of AAV encapsidated sp7-GLA-co4 (AAV-sp7-GLA-co4). Sera were collected weekly; levels of lyso-GL3 were analyzed by mass spectrometry, and GLA activity levels were measured using an in vitro 4-MU-Gal assay. As shown in FIG. 8A, a dose dependent response was observed in the levels of a biomarker of Fabry disease (lyso-GL3) over the course of 28 days of the study. In addition, a linear relationship was observed between serum alpha-Gal A activity and lyso-GL3 levels (FIG. 8B). Dose-dependent decreases in Lyso-GL3 were observed in serum (FIG. 9A) and clinically relevant tissues of kidney (FIG. 9B) and heart (FIG. 9C).

### Example 9—Non-Human Primate (NHP) Study of AAV-Sp7-GLA-Co4

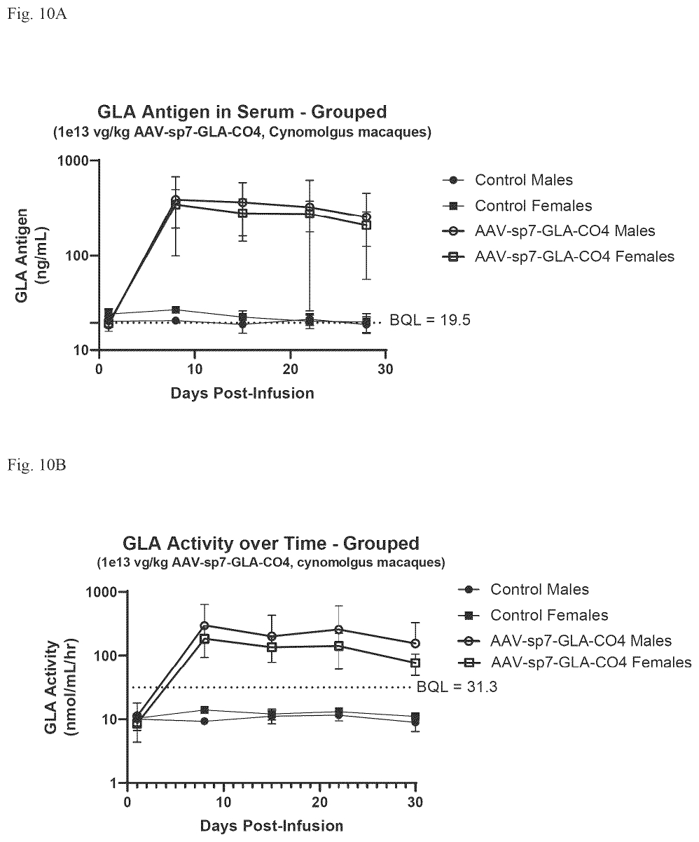

Four male and four female cynomolgus macaques were dosed with AAV-sp7-GLA-co4 at 1e13 vg/kg. control monkeys received vehicle. Sera were collected weekly for 28 days, and assessed for GLA activity levels by the 4-MU-Gal assay (FIG. 10A), and assessed for GLA antigen levels measured using an ELISA specific for human alpha-Gal A (FIG. 10B). As observed, circulating GLA antigen and activity levels in the monkeys that received AAV-sp7-GLA-CO4 were significantly above control levels.

### Example 10—Dose Escalation NHP Study of AAV-Sp7-GLA-Co4

A 60-day GLP-compliant dose finding study is carried out in cynomolgus macaques. The duration of the study is intended to provide a sufficient window to determine the peak expression and detect any potential safety signals. AAV-sp7-GLA-co4 is administered via a single intravenous (IV) infusion to the NHPs in the groups and at the dosages indicated in Table 3 below.

TABLE 3

| | Dose | Number |
|---|---|---|
| Group | (vg/kg) | (Male/Female) |
| 1 | Vehicle | 4/4 |
| 2 | 2 × 10¹² | 4/4 |
| 3 | 6 × 10¹² | 4/4 |
| 4 | 2 × 10¹³ | 4/4 |

Serum samples are taken at intervals over the 60 days following administration, and levels of α-GalA antigen, α-GalA activity, and anti-α-GalA IgG are measured. Standard clinical pathology and anatomical pathology panel analyses are performed. Biodistribution and germline transmission are assessed.

### Example 11—Minimum Efficacious Dose Study

A dose ranging study in mice was performed to identify the minimum efficacious dose to significantly decrease the Fabry biomarkers GL-3 and lyso-GL-3. GLA knockout mice (B6; 129-GLA−/−; also referred to as GLAko or GLA KO) (n=20 per group) were injected intravenously (IV) with three doses ranging from 2E11 vg/kg to 2E12 vg/kg of AAV-sp7-GLA (SEQ ID NO: 21). Serum was taken from ten mice weekly for 6 weeks and at weeks 9 and 12 post AAV injection. Five mice from each group were randomly selected for timed takedowns at 1, 3, 6, and 10 months and analyzed for GLA antigen, GLA activity, and Fabry biomarkers GL-3 and lyso-GL3. Levels of GLA activity were measured using an in vitro (4-MU-GAL) assay (data not shown). Levels of GLA antigen were measured using an ELISA specific for alpha-Gal A.

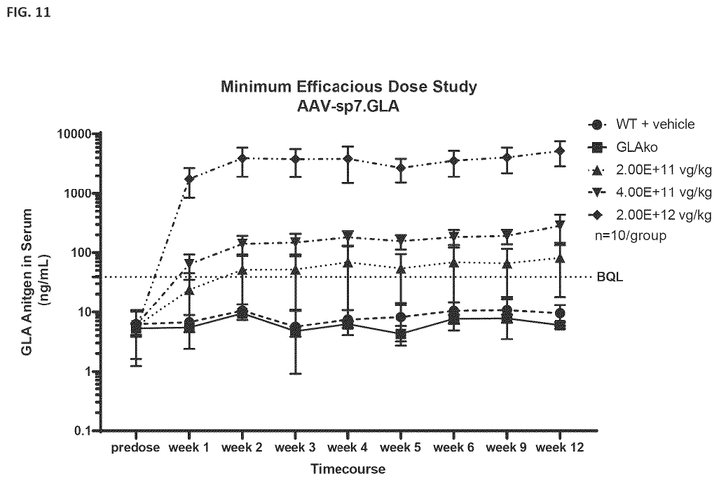

A dose-dependent increase in circulating serum GLA was observed (FIG. 11), with a linear relationship between GLA activity and GLA antigen expression levels. Circulating serum GLA stabilized at week two with 2E11 vg/kg at ˜70 ng/ml, 4E11 vg/kg at ˜190 ng/ml, and 2E12 vg/kg at ˜4 μg/ml (FIG. 11).

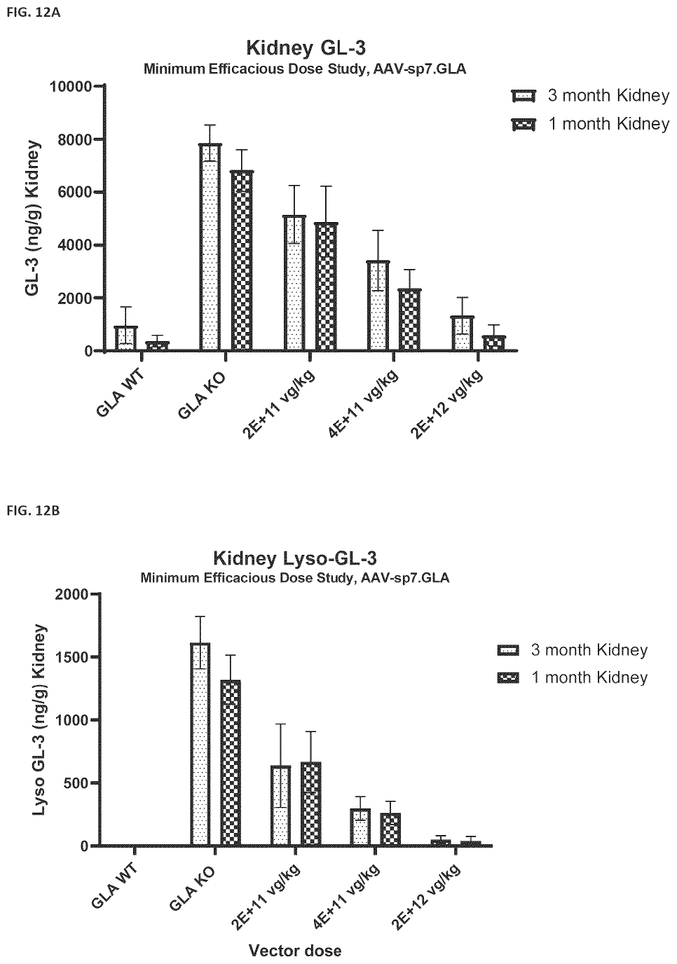

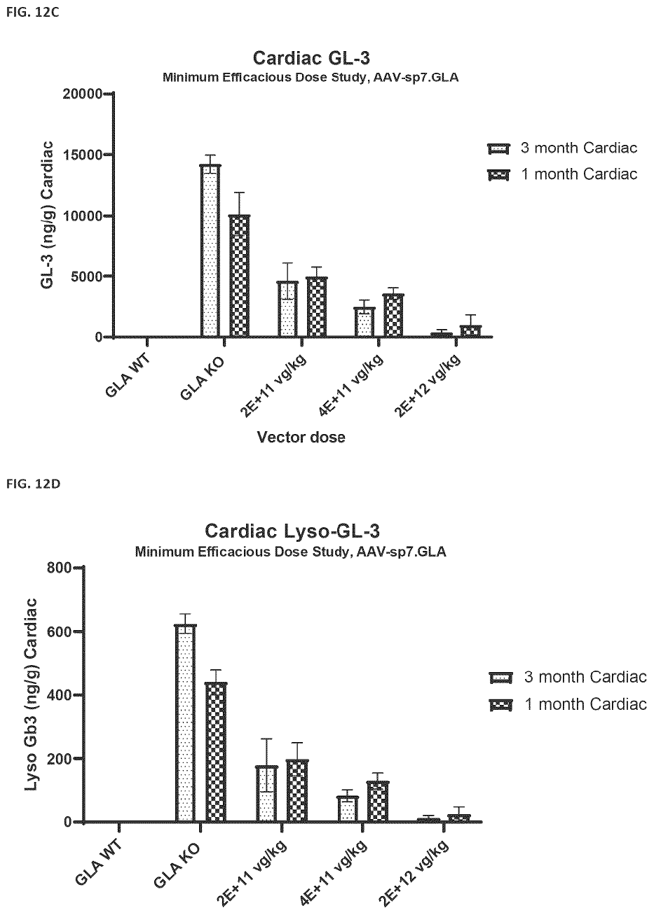

The levels of Fabry biomarkers GL-3 and lyso-GL-3 were measured in the heart and kidney by LC/MS and analyzed using two-way ANOVA with multiple comparisons. Levels of GL3 and lyso-GL-3 in heart and kidney of GLAko mice that received AAV-sp7-GLA at all dosage levels, at both 1 month and 3 month time points, were significantly decreased (p<0.05), compared to GL-3 and lyso-GL-3 levels in the GLAko mice that received vehicle alone (FIGS. 12A-12D).

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, aspects that are not expressly excluded in the instant invention are nevertheless disclosed herein.

I/We claim:

1. A polynucleotide comprising a nucleic acid sequence selected from the group consisting of:
a) a nucleic acid sequence encoding α-galactosidase A (GLA), wherein said nucleic acid sequence is at least 85% identical to the sequence of SEQ ID NO: 15, and wherein said GLA has a sequence at least 95% identical to the sequence of SEQ ID NO: 100;
b) a nucleic acid sequence encoding α-galactosidase A (GLA), wherein said nucleic acid sequence comprises an intron in the coding sequence for said GLA, and wherein said GLA has a sequence at least 95% identical to the sequence of SEQ ID NO: 100;
c) a nucleic acid sequence encoding a precursor α-galactosidase A (GLA) comprising an amino-terminal signal peptide, wherein said signal peptide has a sequence at least 80% identical to a sequence selected from the group consisting of SEQ ID NO: 41, SEQ ID NO: 57, SEQ ID NO: 58, SEQ ID NO: 60, SEQ ID NO: 61, SEQ ID NO: 62, and SEQ ID NO: 63; and wherein said GLA has a sequence at least 95% identical to the sequence of SEQ ID NO: 100; and
d) a nucleic acid sequence encoding an α-galactosidase A (GLA) having an amino acid sequence differing from SEQ ID NO: 100 by 1 to 7 amino acids, wherein at least one of said 1 to 7 amino acids is a substitution selected from the group consisting of Gln57Lys, Gln111Glu, Lys213Glu, Lys237Gln, Phe248Thr, Gly334Glu, and Gly346Asn.

2. A polynucleotide comprising a nucleic acid sequence encoding α-galactosidase A (GLA), wherein said nucleic acid sequence is selected from the group consisting of: (1) a nucleic acid sequence having at least 85% sequence identity to the sequence of SEQ ID NO: 15, (2) a nucleic acid sequence having at least 84% sequence identity to the sequence of SEQ ID NO: 16, (3) a nucleic acid sequence having at least 86% sequence identity to the sequence of SEQ ID NO: 17, (4) a nucleic acid sequence having at least 86% sequence identity to the sequence of SEQ ID NO: 18, and (5) a nucleic acid sequence having at least 83% sequence identity to the sequence of SEQ ID NO: 19.

3. The polynucleotide of claim 1 or 2, wherein said polynucleotide comprises a nucleic acid sequence having at least 95% identity to the sequence of SEQ ID NO: 15.

4. The polynucleotide of claim 3, wherein said nucleic acid sequence encodes GLA comprising the sequence of SEQ ID NO: 100.

5. The polynucleotide of any one of claims 1-4, wherein said nucleic acid sequence contains fewer than 14 CpG dinucleotides.

6. The polynucleotide of claim 1 or 2, wherein said nucleic acid sequence has a sequence of any one of SEQ ID NOs: 15-19, or bases 1-1194 of any one of SEQ ID NOs: 15-19.

7. The polynucleotide of claim 1, wherein said nucleic acid sequence has a sequence at least 95% identical to the sequence of SEQ ID NO: 15, and wherein said GLA has a sequence at least 98% identical to the sequence of SEQ ID NO: 100.

8. The polynucleotide of claim 1, wherein said nucleic acid sequence encodes a GLA protein having an amino acid sequence of SEQ ID NO: 100 having one or more amino acid substitutions selected from the group consisting of Gln57Lys, Gln111Glu, Lys213Glu, Lys237Gln, Phe248Thr, Gly334Glu, and Gly346Asn.

9. The polynucleotide of claim 8, wherein said GLA comprises the amino acid sequence of SEQ ID NO: 48.

10. The polynucleotide of claim 9, wherein the nucleic acid sequence comprises the sequence of SEQ ID NO: 47 or bases 1-1194 of SEQ ID NO: 47.

11. The polynucleotide of claim 1, wherein said nucleic acid sequence encodes said GLA comprising said inserted intron, wherein said GLA in the absence of said intron comprises the GLA sequence provided by any of claims 1-10, wherein said intron is positioned between nucleotides 78 and 79 of the nucleic encoding the GLA and the nucleotide positions are given in reference to the coding sequence of GLA having a sequence of SEQ ID NO: 14.

12. The polynucleotide of claim 11, wherein said intron comprises a sequence selected from the group consisting of SEQ NO: 49, SEQ ID NO: 50, SEQ ID NO: 51 and SEQ ID NO: 52.

13. The polynucleotide of claim 12, wherein the nucleic acid has a sequence of any one of SEQ ID NOs: 43-46.

14. The polynucleotide of any one of claims 1-13, wherein said polynucleotide further comprises a second nucleic acid sequence, wherein said second sequence encodes a signal peptide sequence positioned at the 5′ end of the first nucleic acid sequence.

15. The polynucleotide of claim 14, wherein said signal peptide sequence is a heterologous signal peptide sequence.

16. The polynucleotide of claim 14, wherein said signal peptide sequence is selected from the group consisting of human chymotrypsinogen B2 signal peptide (SEQ ID NO: 41), AHSG signal peptide (SEQ ID NO: 57), CD300 signal peptide (SEQ ID NO: 58), GLA signal peptide (SEQ ID NO: 59) LAMP1 signal peptide (SEQ ID NO: 60), Notch 2 signal peptide (SEQ ID NO: 61), ORM1 signal peptide (SEQ ID NO: 62), TF signal peptide (SEQ ID NO: 63), and wild-type GLA signal peptide (SEQ ID NO: 59), or a variant thereof having a sequence at least 90% identical to the sequence of any of SEQ ID NOs: 41, and 57-63.

17. The polynucleotide of claim 16, wherein said signal peptide is human chymotrypsinogen B2 signal peptide (SEQ ID NO: 41).

18. The polynucleotide of claim 17, wherein said nucleic acid encoding said signal peptide has a sequence of any one of SEQ ID NOs: 1-5.

19. The polynucleotide of any one of claims 1-18, wherein said polynucleotide further comprises an AAV inverted repeat (ITR) flanking the 5′ terminus of said polynucleotide and/or an AAV ITR flanking the 3′ terminus of said polynucleotide.

20. An expression cassette comprising the polynucleotide of any one of claims 1-19, wherein said GLA encoding sequence is operably linked to an expression control element.

21. The expression cassette of claim 20, wherein the expression control element is a liver-specific expression control element.

22. The expression cassette of claim 20 or 21, wherein the expression control element is positioned 5′ of the nucleic acid encoding GLA.

23. The expression cassette of any one of claims 20-22, further comprising a poly-adenylation sequence positioned 3′ of the nucleic acid encoding GLA.

24. The expression cassette of any one of claims 20-23, wherein the expression control element and/or poly-adenylation sequence is CpG-reduced compared to the wild-type expression control element or polyadenylation sequence.

25. The expression cassette of any one of claims 20-24, wherein the expression control element comprises an ApoE/hAAT enhancer/promoter sequence.

26. The expression cassette of any one of claims 23-25, wherein the poly-adenylation sequence comprises a bovine growth hormone (bGH) polyadenylation sequence.

27. The expression cassette of claim 25 or 26, wherein the ApoE/hAAT enhancer/promoter sequence is CpG-reduced compared to wild-type ApoE/hAAT enhancer/promoter sequence.

28. The expression cassette of claim 27, wherein the ApoE/hAAT enhancer/promoter sequence comprises the sequence of SEQ ID NO: 38.

29. The expression cassette of claim 28, wherein the bGH polyadenylation sequence comprises the sequence of SEQ ID NO: 20.

30. The expression cassette of any one of claims 20-29, further comprising an intron positioned between the 3′ end of the expression control element and the 5′ end of the polynucleotide.

31. The expression cassette of claim 30, wherein the intron comprises an hBB2m1 intron (SEQ ID NO: 39).

32. The expression cassette of any one of claims 20-31, wherein said expression cassette further comprises an ITR flanking the 5′ of said expression cassette and/or an ITR flanking the 3′ terminus of said expression cassette.

33. An AAV plasmid genome comprising the polynucleotide of claim 19 or the expression cassette of claim 32, and an origin of replication.

34. An adeno-associated virus (AAV) vector comprising the polynucleotide of claim 19 or the expression cassette of claim 32 and a capsid.

35. The AAV vector of claim 34, wherein said AAV comprises said expression cassette, and said expression cassette further comprises said ITR flanking the 5′ of said expression cassette and said ITR flanking the 3′ terminus of said expression cassette.

36. The AAV vector of claim 34 or 35, wherein said 5′ ITR and said 3′ ITR are independently selected from the group consisting of a AAV1, AAV2, AAV3, AAV4, AAV5, AAV6, AAV7, AAV8, AAV9, AAV10, AAV11, AAV12, Rh10, Rh74 and AAV3B ITR.

37. The AAV vector claim 35 or 36, wherein at least one or more of said 5′ ITR and 3′ ITR is modified to have reduced CpGs.

38. The AAV vector of claim 34, wherein said vector comprises a sequence having a sequence identity of at least 95% with a sequence of any one of SEQ ID NOs: 21-34, 53-56 and 91-99.

39. The AAV vector of claim 34, wherein said vector consists of a sequence having a sequence identity of at least 95% with a sequence of any one of SEQ ID NOs: 91-95.

40. The AAV vector of any one of claims 34-39, wherein said capsid comprises a AAV VP1, VP2 or VP3 capsid having 90% or more sequence identity to VP1, VP2 or VP3 capsid of AAV1, AAV2, AAV3, AAV4, AAV5, AAV6, AAV7, AAV8, AAV9, AAV10, AAV11, AAV12, Rh10, Rh74 (SEQ ID NO: 35), AAV3B, LK03 (SEQ ID NO: 42), AAV-2i8; or SEQ ID NO: 110, SEQ ID NO: 36 or SEQ ID NO: 37.

41. The AAV vector of claim 40, wherein said capsid is a AAV1, AAV2, AAV3, AAV4, AAV5, AAV6, AAV7, AAV8, AAV9, AAV10, AAV11, AAV12, Rh10, Rh74 (SEQ ID NO: 35), AAV3B, LK03 (SEQ ID NO: 42) or AAV-2i8 capsid; or a capsid comprising SEQ ID NO: 110, SEQ ID NO: 36, and/or SEQ ID NO: 37.

42. A pharmaceutical composition comprising a plurality of AAV vector of any one of claims 34-41 in a biologically compatible carrier or excipient.

43. The pharmaceutical composition of claim 42, wherein said plurality of AAV vectors particles is an effective amount to increase GLA activity in a human subject.

44. The pharmaceutical composition of claim 43, further comprising empty AAV capsids.

45. The pharmaceutical composition of claim 44, wherein the ratio of empty AAV capsids to the AAV vector is from about 100:1 to about 1:100.

46. The pharmaceutical composition of any one of claims 42-45, further comprising a surfactant.

47. A method of treating a subject in need of α-galactosidase A (GLA), comprising administering to the subject a therapeutically effective amount of the polynucleotide or expression cassette of any one of claims 1-32 or the AAV vector of any one of claims 34-41, or the pharmaceutical composition of any one of claims 42-46, wherein the GLA is expressed in the subject.

48. The method of claim 47, wherein the subject has Fabry disease.

49. The method of claim 47 or 48, wherein said method reduces, decreases or inhibits one or more symptoms of Fabry disease.

50. The method of any one of claims 47-49, wherein the polynucleotide, expression cassette, AAV vector or pharmaceutical composition is administered to the subject intravenously, intraarterially, intra-cavity, intramucosally, or via catheter.

51. The method of any one of claims 47-50, wherein said AAV is administered to the subject in a range from about 1×10⁸ to about 1×10¹⁴ vector genomes per kilogram (vg/kg) of the weight of the subject.

52. The method of any one of claims 47-51, wherein said subject is a human.

53. A cell comprising the polynucleotide or expression cassette of any one of claims 1-32 or AAV plasmid genome of claim 33.

54. A cell that produces the AAV vector of any one of claims 34-41.

55. A method of producing the AAV vector of any one of claims 34-41, comprising (a) introducing an AAV vector genome plasmid of claim 33 into a packaging helper cell; and (b) culturing the helper cell under conditions to produce the AAV vector.

56. A polypeptide selected from the group consisting of:
a) a precursor α-galactosidase A comprising a signal peptide joined to the amino terminus of α-galactosidase A (GLA), wherein said signal peptide has a sequence at least 80% identical to a sequence selected from the group consisting of SEQ ID NO: 41, SEQ ID NO: 57, SEQ ID NO: 58, SEQ ID NO: 60, SEQ ID NO: 61, SEQ ID NO: 62, and SEQ ID NO: 63; and said GLA has a sequence at least 95% identical to SEQ ID NO: 100; and
b) an α-galactosidase A (GLA) having an amino acid sequence differing from SEQ ID NO: 100 by 1 to 7 amino acids, wherein at least one of said 1 to 7 amino acids is a substitution selected from the group consisting of Gln57Lys, Gln111Glu, Lys213Glu, Lys237Gln, Phe248Thr, Gly334Glu, and Gly346Asn.

57. The polynucleotide of any one of claims 1-19, the expression cassette of any one of claims 20-32, the AAV vector of any one of claims 34-41, the pharmaceutical composition of any one of claims 42-46, or the polypeptide of claim 56, for use in the method of any one of claims 47-52.


SEQUENCE LISTING (110 sequences)

SEQ ID NO: 1 - DNA, 54 bp, Artificial Sequence
  sp7 signal peptide variant
atggccttcctgtggctgctgtcctgctgggcactgctgggcaccacatttggc

SEQ ID NO: 2 - DNA, 54 bp, Artificial Sequence
  sp7 signal peptide variant
atggcctttctgtggctgctgtcctgctgggccctgctggggaccacctttggc

SEQ ID NO: 3 - DNA, 54 bp, Artificial Sequence
  sp7 signal peptide variant
atggccttcctgtggctgctgagctgctgggccctgctggggaccacctttggg

SEQ ID NO: 4 - DNA, 54 bp, Artificial Sequence
  sp7 signal peptide variant
atggccttcctgtggctcctgagctgctgggccctcctggggaccacctttggg

SEQ ID NO: 5 - DNA, 54 bp, Artificial Sequence
  sp7 signal peptide variant
atggccttcctgtggctgctgagctgctgggccctgctggggaccacatttggc

SEQ ID NO: 6 - DNA, 54 bp, Artificial Sequence
  AHSG signal peptide
atgaagtccctcgtcctgctcctttgtcttgctcagctctggggctgccactca

SEQ ID NO: 7 - DNA, 51 bp, Artificial Sequence
  CD300 signal peptide
atgtggctgccttgggctctgttgcttctctgggtcccaggatgttttgct

SEQ ID NO: 8 - DNA, 93 bp, Artificial Sequence
  GLA signal peptide
atgcagctgaggaacccagaactacatctgggctgcgcgcttgcgcttcgcttcctggccctcgtttcctgggacatccctggggctagagca

SEQ ID NO: 9 - DNA, 84 bp, Artificial Sequence
  LAMP1 signal peptide
atggcggcccccggcagcgcccggcgacccctgctgctgctactgctgttgctgctgctcggcctcatgcattgtgcgtcagca

SEQ ID NO: 10 - DNA, 75 bp, Artificial Sequence
  Notch2 signal peptide
atgcccgccctgcgccccgctctgctgtgggcgctgctggcgctctggctgtgctgcgcggcccccgcgcatgca

SEQ ID NO: 11 - DNA, 54 bp, Artificial Sequence
  ORM1 signal peptide
atggcgctgtcctgggttcttacagtcctgagcctcctacctctgctggaagcc

SEQ ID NO: 12 - protein, 429 aa, Artificial Sequence
  full-length GLA protein sequence
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 13 - DNA, 57 bp, Artificial Sequence
  TF signal peptide
atgaggctcgccgtgggagccctgctggtctgcgccgtcctggggctgtgtctggct

SEQ ID NO: 14 - DNA, 1197 bp, Artificial Sequence
  Partial GLA cDNA, GLA coding sequence
ctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 15 - DNA, 1200 bp, Artificial Sequence
  Partial GLA cDNA, GLA coding sequence, GLAco4 variant
ctggacaatggcctggccaggacccccaccatggggtggctgcactgggagaggttcatgtgcaacctggattgccaggaggagccagattcttgcatctctgagaagctgttcatggagatggctgagctgatggtgtctgagggctggaaggatgctggctatgagtacctgtgcattgatgactgctggatggctccccagagggactctgagggcagactgcaggctgacccccagaggttcccccatgggatcaggcagctggccaactatgtgcacagcaagggcctgaagctgggcatttatgctgatgtgggcaacaagacctgtgctggcttccctggctcttttggctattatgacattgatgctcagacctttgctgactggggggtggacctgctgaagtttgatgggtgctattgtgactctctggagaacctggctgatggctataagcatatgtctctggccctgaacaggactggcaggagcattgtctacagctgtgagtggcctctgtacatgtggcctttccagaagcctaactacactgagatcaggcagtattgcaaccactggaggaactttgctgacattgatgactcttggaagtctattaagagcattctggattggaccagcttcaaccaggaaaggattgtggatgtggctgggcctgggggctggaatgaccctgacatgctggtgattgggaactttggcctgagctggaaccagcaggtgacccagatggccctgtgggccatcatggctgcccccctgttcatgtccaatgacctgaggcacatcagcccccaggccaaggccctgctgcaggataaggatgtgattgccatcaatcaggaccccctggggaagcagggctaccagctgaggcagggggacaactttgaggtgtgggagagacctctgtctgggctggcctgggctgtggccatgatcaacaggcaggagattgggggccccaggagctataccattgctgtggcctctctggggaagggggtggcctgcaaccctgcctgcttcatcacccagctgctgcctgtgaagaggaagctgggcttctatgagtggaccagcaggctgaggagccacattaatcccactggcactgtgctgctgcagctggagaacaccatgcagatgtctctgaaggatctgctgtgataa

SEQ ID NO: 16 - DNA, 1197 bp, Artificial Sequence
  Partial GLA cDNA, GLA coding sequence, GLAcoBC0 variant
ctggacaatggcctggccagaacccccaccatgggctggctgcactgggagaggttcatgtgcaaccttgactgccaggaggagcctgacagctgcatctctgagaagctgttcatggagatggctgagctcatggtgtcagaggggtggaaggatgcaggctatgagtacctgtgcatagatgactgctggatggctccccagagggactcagagggcaggctgcaggctgaccctcagaggttcccccatggcatcaggcagctggccaactatgtgcacagcaagggcctcaagctgggaatctatgctgatgtgggcaacaaaacatgtgcaggcttcccaggctcttttggctactatgacatagatgcccagacctttgcagactggggagttgacctgctgaagtttgatgggtgctactgtgacagcctggagaacctggctgatgggtacaagcacatgtccctggccctgaacaggacaggcaggtccattgtgtacagctgtgagtggcccctgtacatgtggcccttccagaagcccaactacacagagatcagacagtactgcaaccactggagaaactttgctgacattgatgactcctggaagagcatcaagtccatcctggactggaccagcttcaaccaggagaggattgtggatgtggctggcccagggggctggaatgaccctgacatgctggtcattggcaactttggcctgagctggaaccagcaggtgacccagatggccctgtgggccatcatggctgcccccctcttcatgagcaatgacctgagacacatctccccccaagccaaggccctgttgcaggacaaggatgtgattgccatcaatcaggaccccttgggcaagcagggctaccagctgagacagggggacaactttgaggtgtgggagagacccctctctggcttggcctgggctgtggccatgatcaacagacaggagattgggggccccagaagctacaccattgctgttgccagcctgggcaagggggtggcctgcaaccctgcctgcttcatcacacagctgctgcctgtgaagaggaagctggggttctatgagtggacctctaggctgagaagtcacatcaaccccacaggcacagtgctgctgcagttggagaacaccatgcagatgagcctgaaggacctgctctga

SEQ ID NO: 17 - DNA, 1197 bp, Artificial Sequence
  Partial GLA cDNA, GLA coding sequence, GLAcoH0 variant
ctggacaatggtctggccaggactcccaccatgggctggctgcactgggagaggttcatgtgcaacctggactgccaggaggagccagactcctgcatcagtgagaagctcttcatggagatggctgagctcatggtctctgaagggtggaaggatgctgggtatgagtacctgtgtattgatgactgctggatggccccccaaagggactcagagggcaggctgcaggcagaccctcagaggttccctcatgggatcaggcagcttgccaactatgtccacagcaaggggctgaagctggggatctatgctgatgttgggaacaagacctgtgctggcttcccaggctcctttggctactatgatattgatgcacagacatttgcagattggggggtggatctgctgaagtttgatgggtgctactgtgactccctggagaaccttgcagatgggtacaagcacatgtccctggccctgaacaggactggcagaagcatagtctactcctgtgagtggcccctgtacatgtggcccttccagaagcccaactacacagagatcagacagtactgcaaccactggaggaactttgcagacattgatgattcctggaagagtatcaagagcatcctagactggacaagctttaaccaggagaggattgtggatgtagcaggcccagggggctggaatgacccagacatgcttgtgattggcaactttggcctcagctggaaccagcaggtgactcagatggccctgtgggccatcatggctgcccccctgttcatgtctaatgacctcaggcacatcagcccacaagccaaggcactcctccaggacaaggatgtgatagccatcaaccaggaccccttggggaagcaggggtaccagctaaggcagggggacaactttgaggtgtgggaaaggcccctctcagggcttgcctgggcagtagctatgatcaacaggcaggagattggtggccccagatcttacactattgcagtggctagcctggggaagggggtggcctgcaaccctgcctgcttcatcacacagctcctcccagtcaagaggaagttggggttctatgagtggacttccaggctgagatcccacatcaaccccacaggcactgtgctgctgcagctagagaacaccatgcagatgagcctgaaggacctcctctga

SEQ ID NO: 18 - DNA, 1197 bp, Artificial Sequence
  Partial GLA cDNA, GLA coding sequence, GLAcoH6 variant
ctggacaatggcttggccaggactcccaccatgggctggctgcactgggagaggttcatgtgcaacttggactgccaggaggagccagactcctgcatctctgagaagctcttcatggagatggctgagctcatggtctcagaggggtggaaggatgcagggtatgagtacctctgcattgatgactgctggatggcccctcaaagggattcagagggtaggctccaggcagacccccagaggttcccccatggcatcaggcagcttgctaactatgtccacagcaagggcctgaagctggggatctatgctgatgttggcaacaagacctgtgctggcttcccaggttcctttgggtactatgacattgatgcacagacctttgcagactggggggtagacctcctaaagtttgatgggtgctactgtgacagcttggagaacctggcagatggctacaagcacatgagccttgccctgaacaggactggcagaagcatagtgtactcctgtgagtggcccctgtacatgtggcccttccagaagcccaactacacagagatcagacagtactgcaaccactggaggaactttgctgacatagatgactcctggaagagcataaagagcatcctggactggacatccttcaaccaggagaggattgtggatgtggctgggccagggggctggaatgacccagacatgcttgtgataggcaactttggcctcagctggaaccagcaggtgactcagatggccctctgggccatcatggctgcccccctgttcatgagcaatgacctcaggcacatcagcccccaagccaaggccctcctccaggacaaggatgtgatagccatcaatcaggaccccctaggcaagcaggggtaccagctcagacagggtgacaactttgaggtgtgggaaaggcctctgtcaggccttgcctgggcagtggctatgatcaacaggcaggagattggtgggcctaggagctacaccattgcagtggcctccctggggaagggagtggcctgcaacccagcctgcttcatcacacagctcctccctgtgaagaggaagcttggcttctatgaatggacctccaggctgaggagtcacatcaaccccacagggactgtcctcctacagctagagaacaccatgcagatgtccctgaaggacctgctgtga

SEQ ID NO: 19 - DNA, 1197 bp, Artificial Sequence
  Partial GLA cDNA, GLA coding sequence, GLAvar45v2 variant
ctggacaatggcctggccaggacccccaccatgggctggctgcactgggagagattcatgtgcaacctggactgccaggaggagcctgacagctgcatctctgagaagctgttcatggagatggcagagctgatggtgtctgagggctggaaggatgctggctatgagtacctgtgcattgatgactgctggatggccccccagagagactctgagggcaggctgcaggcagacccccagaggttcccccatggcatcagacagctggccaactatgtgcacagcaagggcctgaagctgggcatctatgctgatgtgggcaacaagacctgtgcaggcttccctggctcctttggctactatgacattgatgcccagacctttgctgactggggagtggacctgctgaagtttgatggctgctactgtgactccctggagaacctggctgatggctacaagcacatgtccctggccctgaacaggacaggcagatccattgtgtacagctgtgagtggcccctgtacatgtggccattccagaagcccaactacacagagatcaggcagtactgcaaccactggagaaactttgcagacattgatgattcctggaagagcatcaagtccatcctggactggacctccttcaaccaggagagaattgtggatgtggcaggccctgggggctggaatgacccagacatgctggtcattggcaactttggcctgtcctggaaccagcaggtgacccagatggccctgtgggccatcatggcagcccccctgttcatgagcaatgacctgagacacatcagcccacaggccaaggccctgctgcaggacaaggatgtgattgccatcaaccaggaccctctgggcaagcagggctaccagctgaggcagggagacaactttgaggtgtgggagaggcccctgtctggcctggcctgggcagtggccatgatcaacaggcaggagattggaggccccaggagctacaccattgcagtggccagcctgggcaagggggtggcctgcaacccagcctgcttcatcacccagctgctgcctgtgaagaggaagctgggcttctatgagtggacctccagactgaggagccacatcaaccccacaggcacagtgctgctgcagctggagaacaccatgcagatgagcctgaaggacctgctgtga

SEQ ID NO: 20 - DNA, 228 bp, Artificial Sequence
  BGH228 polyA sequence
ctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 21 - DNA, 3122 bp, Artificial Sequence
  pAAV_ApoE_hAAT_HBB2m1_sp7.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctgctgtcctgctgggcactgctgggcaccacatttggcctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 3122 bp, Artificial Sequence
  pAAV_ApoE_hAAT_HBB2m1_spAHSG.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgaagtccctcgtcctgctcctttgtcttgctcagctctggggctgccactcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 3119 bp, Artificial Sequence
  pAAV_ApoE_hAAT_HBB2m1_spCD300.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgtggctgccttgggctctgttgcttctctgggtcccaggatgttttgctctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 3161 bp, Artificial Sequence
  pAAV_ApoE_hAAT_HBB2m1_spGLA.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - DNA, 3152 bp, Artificial Sequence
  pAAV_ApoE_hAAT_HBB2m1_spLAMP1.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcggcccccggcagcgcccggcgacccctgctgctgctactgctgttgctgctgctcggcctcatgcattgtgcgtcagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 26 - DNA, 3143 bp, Artificial Sequence
  pAAV_ApoE_hAAT_HBB2m1_spNotch2.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgcccgccctgcgccccgctctgctgtgggcgctgctggcgctctggctgtgctgcgcggcccccgcgcatgcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 27 - DNA, 3122 bp, Artificial Sequence
  pAAV_ApoE_hAAT_HBB2m1_spORM1.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcgctgtcctgggttcttacagtcctgagcctcctacctctgctggaagccctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 28 - DNA, 3122 bp, Artificial Sequence
  pSPKL0031 (GLA 7 mut)
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgcaaagaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctgaacgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaagaacccaattatacagaaatccgacagtactgcaatcactggcgaaattttgctgacattgatgattcctggcagagtataaagagtatcttggactggacatctaccaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacaggaagacaactttgaagtgtgggaacgacctctctcaaacttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 29 - DNA, 3125 bp, Artificial Sequence
  pAAV_ApoE_hAAT_HBB2m1_spTF.GLA_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatgaggctcgccgtgggagccctgctggtctgcgccgtcctggggctgtgtctggctctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - DNA, 3147 bp, Artificial Sequence
  pAAV_ApoE_hAAT_HBB2m1_sp7.GLAco4_BGH228 sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgtcctgctgggccctgctggggaccacctttggcctggacaatggcctggccaggacccccaccatggggtggctgcactgggagaggttcatgtgcaacctggattgccaggaggagccagattcttgcatctctgagaagctgttcatggagatggctgagctgatggtgtctgagggctggaaggatgctggctatgagtacctgtgcattgatgactgctggatggctccccagagggactctgagggcagactgcaggctgacccccagaggttcccccatgggatcaggcagctggccaactatgtgcacagcaagggcctgaagctgggcatttatgctgatgtgggcaacaagacctgtgctggcttccctggctcttttggctattatgacattgatgctcagacctttgctgactggggggtggacctgctgaagtttgatgggtgctattgtgactctctggagaacctggctgatggctataagcatatgtctctggccctgaacaggactggcaggagcattgtctacagctgtgagtggcctctgtacatgtggcctttccagaagcctaactacactgagatcaggcagtattgcaaccactggaggaactttgctgacattgatgactcttggaagtctattaagagcattctggattggaccagcttcaaccaggaaaggattgtggatgtggctgggcctgggggctggaatgaccctgacatgctggtgattgggaactttggcctgagctggaaccagcaggtgacccagatggccctgtgggccatcatggctgcccccctgttcatgtccaatgacctgaggcacatcagcccccaggccaaggccctgctgcaggataaggatgtgattgccatcaatcaggaccccctggggaagcagggctaccagctgaggcagggggacaactttgaggtgtgggagagacctctgtctgggctggcctgggctgtggccatgatcaacaggcaggagattgggggccccaggagctataccattgctgtggcctctctggggaagggggtggcctgcaaccctgcctgcttcatcacccagctgctgcctgtgaagaggaagctgggcttctatgagtggaccagcaggctgaggagccacattaatcccactggcactgtgctgctgcagctggagaacaccatgcagatgtctctgaaggatctgctgtgataaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 31 - DNA, 3144 bp, Artificial Sequence
  pAAV_ApoE_hAAT_HBB2m1_sp7.GLAcoBC0_BGH228 sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctgctgagctgctgggccctgctggggaccacctttgggctggacaatggcctggccagaacccccaccatgggctggctgcactgggagaggttcatgtgcaaccttgactgccaggaggagcctgacagctgcatctctgagaagctgttcatggagatggctgagctcatggtgtcagaggggtggaaggatgcaggctatgagtacctgtgcatagatgactgctggatggctccccagagggactcagagggcaggctgcaggctgaccctcagaggttcccccatggcatcaggcagctggccaactatgtgcacagcaagggcctcaagctgggaatctatgctgatgtgggcaacaaaacatgtgcaggcttcccaggctcttttggctactatgacatagatgcccagacctttgcagactggggagttgacctgctgaagtttgatgggtgctactgtgacagcctggagaacctggctgatgggtacaagcacatgtccctggccctgaacaggacaggcaggtccattgtgtacagctgtgagtggcccctgtacatgtggcccttccagaagcccaactacacagagatcagacagtactgcaaccactggagaaactttgctgacattgatgactcctggaagagcatcaagtccatcctggactggaccagcttcaaccaggagaggattgtggatgtggctggcccagggggctggaatgaccctgacatgctggtcattggcaactttggcctgagctggaaccagcaggtgacccagatggccctgtgggccatcatggctgcccccctcttcatgagcaatgacctgagacacatctccccccaagccaaggccctgttgcaggacaaggatgtgattgccatcaatcaggaccccttgggcaagcagggctaccagctgagacagggggacaactttgaggtgtgggagagacccctctctggcttggcctgggctgtggccatgatcaacagacaggagattgggggccccagaagctacaccattgctgttgccagcctgggcaagggggtggcctgcaaccctgcctgcttcatcacacagctgctgcctgtgaagaggaagctggggttctatgagtggacctctaggctgagaagtcacatcaaccccacaggcacagtgctgctgcagttggagaacaccatgcagatgagcctgaaggacctgctctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - DNA, 3144 bp, Artificial Sequence
  pAAV_ApoE_hAAT_HBB2m1_sp7.GLAcoH0_BGH228 sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctcctgagctgctgggccctcctggggaccacctttgggctggacaatggtctggccaggactcccaccatgggctggctgcactgggagaggttcatgtgcaacctggactgccaggaggagccagactcctgcatcagtgagaagctcttcatggagatggctgagctcatggtctctgaagggtggaaggatgctgggtatgagtacctgtgtattgatgactgctggatggccccccaaagggactcagagggcaggctgcaggcagaccctcagaggttccctcatgggatcaggcagcttgccaactatgtccacagcaaggggctgaagctggggatctatgctgatgttgggaacaagacctgtgctggcttcccaggctcctttggctactatgatattgatgcacagacatttgcagattggggggtggatctgctgaagtttgatgggtgctactgtgactccctggagaaccttgcagatgggtacaagcacatgtccctggccctgaacaggactggcagaagcatagtctactcctgtgagtggcccctgtacatgtggcccttccagaagcccaactacacagagatcagacagtactgcaaccactggaggaactttgcagacattgatgattcctggaagagtatcaagagcatcctagactggacaagctttaaccaggagaggattgtggatgtagcaggcccagggggctggaatgacccagacatgcttgtgattggcaactttggcctcagctggaaccagcaggtgactcagatggccctgtgggccatcatggctgcccccctgttcatgtctaatgacctcaggcacatcagcccacaagccaaggcactcctccaggacaaggatgtgatagccatcaaccaggaccccttggggaagcaggggtaccagctaaggcagggggacaactttgaggtgtgggaaaggcccctctcagggcttgcctgggcagtagctatgatcaacaggcaggagattggtggccccagatcttacactattgcagtggctagcctggggaagggggtggcctgcaaccctgcctgcttcatcacacagctcctcccagtcaagaggaagttggggttctatgagtggacttccaggctgagatcccacatcaaccccacaggcactgtgctgctgcagctagagaacaccatgcagatgagcctgaaggacctcctctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 3144 bp, Artificial Sequence
  pAAV_ApoE_hAAT_HBB2m1_sp7.GLAcoH6_BGH228 sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctgctgagctgctgggccctgctggggaccacatttggcctggacaatggcttggccaggactcccaccatgggctggctgcactgggagaggttcatgtgcaacttggactgccaggaggagccagactcctgcatctctgagaagctcttcatggagatggctgagctcatggtctcagaggggtggaaggatgcagggtatgagtacctctgcattgatgactgctggatggcccctcaaagggattcagagggtaggctccaggcagacccccagaggttcccccatggcatcaggcagcttgctaactatgtccacagcaagggcctgaagctggggatctatgctgatgttggcaacaagacctgtgctggcttcccaggttcctttgggtactatgacattgatgcacagacctttgcagactggggggtagacctcctaaagtttgatgggtgctactgtgacagcttggagaacctggcagatggctacaagcacatgagccttgccctgaacaggactggcagaagcatagtgtactcctgtgagtggcccctgtacatgtggcccttccagaagcccaactacacagagatcagacagtactgcaaccactggaggaactttgctgacatagatgactcctggaagagcataaagagcatcctggactggacatccttcaaccaggagaggattgtggatgtggctgggccagggggctggaatgacccagacatgcttgtgataggcaactttggcctcagctggaaccagcaggtgactcagatggccctctgggccatcatggctgcccccctgttcatgagcaatgacctcaggcacatcagcccccaagccaaggccctcctccaggacaaggatgtgatagccatcaatcaggaccccctaggcaagcaggggtaccagctcagacagggtgacaactttgaggtgtgggaaaggcctctgtcaggccttgcctgggcagtggctatgatcaacaggcaggagattggtgggcctaggagctacaccattgcagtggcctccctggggaagggagtggcctgcaacccagcctgcttcatcacacagctcctccctgtgaagaggaagcttggcttctatgaatggacctccaggctgaggagtcacatcaaccccacagggactgtcctcctacagctagagaacaccatgcagatgtccctgaaggacctgctgtgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 34 - DNA, 3139 bp, Artificial Sequence
  pAAV_ApoE_hAAT_HBB2m1_sp7.GLAvar45v2_BGH228 sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcagccaccatggcctttctgtggctgctgtcctgctgggccctgctggggaccacctttggcctggacaatggcctggccaggacccccaccatgggctggctgcactgggagagattcatgtgcaacctggactgccaggaggagcctgacagctgcatctctgagaagctgttcatggagatggcagagctgatggtgtctgagggctggaaggatgctggctatgagtacctgtgcattgatgactgctggatggccccccagagagactctgagggcaggctgcaggcagacccccagaggttcccccatggcatcagacagctggccaactatgtgcacagcaagggcctgaagctgggcatctatgctgatgtgggcaacaagacctgtgcaggcttccctggctcctttggctactatgacattgatgcccagacctttgctgactggggagtggacctgctgaagtttgatggctgctactgtgactccctggagaacctggctgatggctacaagcacatgtccctggccctgaacaggacaggcagatccattgtgtacagctgtgagtggcccctgtacatgtggccattccagaagcccaactacacagagatcaggcagtactgcaaccactggagaaactttgcagacattgatgattcctggaagagcatcaagtccatcctggactggacctccttcaaccaggagagaattgtggatgtggcaggccctgggggctggaatgacccagacatgctggtcattggcaactttggcctgtcctggaaccagcaggtgacccagatggccctgtgggccatcatggcagcccccctgttcatgagcaatgacctgagacacatcagcccacaggccaaggccctgctgcaggacaaggatgtgattgccatcaaccaggaccctctgggcaagcagggctaccagctgaggcagggagacaactttgaggtgtgggagaggcccctgtctggcctggcctgggcagtggccatgatcaacaggcaggagattggaggccccaggagctacaccattgcagtggccagcctgggcaagggggtggcctgcaacccagcctgcttcatcacccagctgctgcctgtgaagaggaagctgggcttctatgagtggacctccagactgaggagccacatcaaccccacaggcacagtgctgctgcagctggagaacaccatgcagatgagcctgaaggacctgctgtgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 35 - protein, 738 aa, Artificial Sequence
  VP1
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 36 - protein, 601 aa, Artificial Sequence
  VP2
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 37 - protein, 535 aa, Artificial Sequence
  VP3
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 38 - DNA, 727 bp, Artificial Sequence
  ApoE/hAAT promoter/enhancer sequence
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 39 - DNA, 543 bp, Artificial Sequence
  hBB2m1 intron sequence
gatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 40 - protein, 388 aa, Artificial Sequence [sequence omitted]

SEQ ID NO: 41 - protein, 18 aa, Artificial Sequence
  SP7
MAFLWLLSCWALLGTTFG

SEQ ID NO: 42 - protein, 736 aa, Artificial Sequence
  VP1
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 43 - DNA, 1658 bp, Artificial Sequence
  GLA-int-VTN1
ctggacaatggattggcaaggacgcctaccatgggctggctgcactgggagcgcttcatgtgcaaccttgactgccaggtatggagagagggcaagtcttgcttctccctcaaaagggctgaaaccccttggtattggtagagccaggccggctggagggggctgtggttgtggagctatcgatcaaagtctgtttgctcaggccagactttgcttctgttgaccttttggggaaagctcagctctacctggaccccacaccttggactttgcctagcacagctgagagcacagccagcagagggaggggctgtggctgaggagtttagggggcctgggggggtggggtcgagacaccagtgatatggtggagggaaagcacagggggaagggaattggactgagagtcaaaggcctggctctgccattcgctgctgtgtgtctttgggcaaggtgcagcagatgaactctaatggccccgctggaaggggcaagattcggacccccaagacctctcattcaccccttccctgccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 44 - DNA, 1364 bp, Artificial Sequence
  GLA-int-RBP4
ctggacaatggattggcaaggacgcctaccatgggctggctgcactgggagcgcttcatgtgcaaccttgactgccaggtcagtggccgccggggcagctgcgccctttgcgctccagggttccccaagggccctgcctgctgatcgccacgtgggcattgtgaagggaagggagcaccgaatgggtggagggagggaggaagccctttgcccggcttggctgaggatccccttggcttttgcaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 45 - DNA, 1279 bp, Artificial Sequence
  GLA-int-IgHA
ctggacaatggattggcaaggacgcctaccatgggctggctgcactgggagcgcttcatgtgcaaccttgactgccaggtgagtacaggaggtggagagtggccagcccttctcatgttcagagaacatggttaactggttaagtcatgtcgtcccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 46 - DNA, 1307 bp, Artificial Sequence
  GLA-int-IgHM
ctggacaatggattggcaaggacgcctaccatgggctggctgcactgggagcgcttcatgtgcaaccttgactgccaggtaagaaccaaaccctcccagcaggggtgcccaggcccaggcatggcccagagggagcagcgggtggggcttaggccaagctgagctcacaccttgacctttcattacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 47 - DNA, 1197 bp, Artificial Sequence
  GLA 7 mut
ctggacaatggattggcaaggacgcctaccatgggctggctgcactgggagcgcttcatgtgcaaccttgactgcaaagaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctgaacgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaagaacccaattatacagaaatccgacagtactgcaatcactggcgaaattttgctgacattgatgattcctggcagagtataaagagtatcttggactggacatctaccaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacaggaagacaactttgaagtgtgggaacgacctctctcaaacttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 48 - protein, 398 aa, Artificial Sequence
  GLA 7 mut aa sequence
LDNGLARTPTMGWLHWERFMCNLDCKEEPDSCISEKLFMEMAELMVSEGWKDAGYEYLCIDDCWMAPQRDSEGRLQADPERFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQEPNYTEIRQYCNHWRNFADIDDSWQSIKSILDWTSTNQERIVDVAGPGGWNDPDMLVIGNFGLSWNQQVTQMALWAIMAAPLFMSNDLRHISPQAKALLQDKDVIAINQDPLGKQGYQLRQEDNFEVWERPLSNLAWAVAMINRQEIGGPRSYTIAVASLGKGVACNPACFITQLLPVKRKLGFYEWTSRLRSHINPTGTVLLQLENTMQMSLKDLL

SEQ ID NO: 49 - DNA, 461 bp, Artificial Sequence
  Intron from vitronectin1 gene
gtatggagagagggcaagtcttgcttctccctcaaaagggctgaaaccccttggtattggtagagccaggccggctggagggggctgtggttgtggagctatcgatcaaagtctgtttgctcaggccagactttgcttctgttgaccttttggggaaagctcagctctacctggaccccacaccttggactttgcctagcacagctgagagcacagccagcagagggaggggctgtggctgaggagtttagggggcctgggggggtggggtcgagacaccagtgatatggtggagggaaagcacagggggaagggaattggactgagagtcaaaggcctggctctgccattcgctgctgtgtgtctttgggcaaggtgcagcagatgaactctaatggccccgctggaaggggcaagattcggacccccaagacctctcattcaccccttccctgccacag

SEQ ID NO: 50 - DNA, 167 bp, Artificial Sequence
  Intron from RBP4 gene
gtcagtggccgccggggcagctgcgccctttgcgctccagggttccccaagggccctgcctgctgatcgccacgtgggcattgtgaagggaagggagcaccgaatgggtggagggagggaggaagccctttgcccggcttggctgaggatccccttggcttttgcag

SEQ ID NO: 51 - DNA, 82 bp, Artificial Sequence
  Intron from heavy chain of mouse IgG heavy chain A gene
gtgagtacaggaggtggagagtggccagcccttctcatgttcagagaacatggttaactggttaagtcatgtcgtcccacag

SEQ ID NO: 52 - DNA, 110 bp, Artificial Sequence
  Intron from heavy chain of mouse IgG heavy chain mu gene
gtaagaaccaaaccctcccagcaggggtgcccaggcccaggcatggcccagagggagcagcgggtggggcttaggccaagctgagctcacaccttgacctttcattacag

SEQ ID NO: 53 - DNA, 3226 bp, Artificial Sequence
  AAV_ApoE_HAT_sp7_GLA_WT_intronIgHA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtgagtacaggaggtggagagtggccagcccttctcatgttcagagaacatggttaactggttaagtcatgtcgtcccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 54 - DNA, 3254 bp, Artificial Sequence
  AAV_ApoE_HAT_sp7_GLA_WT_intronIgHmu
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtaagaaccaaaccctcccagcaggggtgcccaggcccaggcatggcccagagggagcagcgggtggggcttaggccaagctgagctcacaccttgacctttcattacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 55 - DNA, 3311 bp, Artificial Sequence
  AAV_ApoE_HAT_sp7_GLA_WT_intronRBP4
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtcagtggccgccggggcagctgcgccctttgcgctccagggttccccaagggccctgcctgctgatcgccacgtgggcattgtgaagggaagggagcaccgaatgggtggagggagggaggaagccctttgcccggcttggctgaggatccccttggcttttgcaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 56 - DNA, 3605 bp, Artificial Sequence
  AAV_ApoE_HAT_sp7_GLA_WT_intronVTN1
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtatggagagagggcaagtcttgcttctccctcaaaagggctgaaaccccttggtattggtagagccaggccggctggagggggctgtggttgtggagctatcgatcaaagtctgtttgctcaggccagactttgcttctgttgaccttttggggaaagctcagctctacctggaccccacaccttggactttgcctagcacagctgagagcacagccagcagagggaggggctgtggctgaggagtttagggggcctgggggggtggggtcgagacaccagtgatatggtggagggaaagcacagggggaagggaattggactgagagtcaaaggcctggctctgccattcgctgctgtgtgtctttgggcaaggtgcagcagatgaactctaatggccccgctggaaggggcaagattcggacccccaagacctctcattcaccccttccctgccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 57 - protein, 18 aa, Artificial Sequence
  AHSG signal peptide aa sequence
MKSLVLLLCLAQLWGCHS

SEQ ID NO: 58 - protein, 17 aa, Artificial Sequence
  CD300 signal peptide aa sequence
MWLPWALLLLWVPGCFA

SEQ ID NO: 59 - protein, 31 aa, Artificial Sequence
  GLA signal peptide aa sequence
MQLRNPELHLGCALALRFLALVSWDIPGARA

SEQ ID NO: 60 - protein, 28 aa, Artificial Sequence
  LAMP1 signal peptide aa sequence
MAAPGSARRPLLLLLLLLLLGLMHCASA

SEQ ID NO: 61 - protein, 25 aa, Artificial Sequence
  Notch2 signal peptide aa sequence
MPALRPALLWALLALWLCCAAPAHA

SEQ ID NO: 62 - protein, 18 aa, Artificial Sequence
  ORM1 signal peptide aa sequence
MALSWVLTVLSLLPLLEA

SEQ ID NO: 63 - protein, 19 aa, Artificial Sequence
  TF signal peptide aa sequence
MRLAVGALLVCAVLGLCLA

SEQ ID NO: 64 - DNA, 1251 bp, Artificial Sequence
  sp7-GLA cDNA
atggcctttctgtggctgctgtcctgctgggccctgctggggaccacctttggcctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 65 - DNA, 1251 bp, Artificial Sequence
  spAHSG-GLA cDNA
atgaagtccctcgtcctgctcctttgtcttgctcagctctggggctgccactcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 66 - DNA, 1248 bp, Artificial Sequence
  spCD300-GLA cDNA
atgtggctgccttgggctctgttgcttctctgggtcccaggatgttttgctctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 67 - DNA, 1290 bp, Artificial Sequence
  spGLA-GLA cDNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 68 - DNA, 1281 bp, Artificial Sequence
  spLAMP1-GLA cDNA
atggcggcccccggcagcgcccggcgacccctgctgctgctactgctgttgctgctgctcggcctcatgcattgtgcgtcagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 69 - DNA, 1272 bp, Artificial Sequence
  spNotch2-GLA cDNA
atgcccgccctgcgccccgctctgctgtgggcgctgctggcgctctggctgtgctgcgcggcccccgcgcatgcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 70 - DNA, 1251 bp, Artificial Sequence
  spORM1-GLA cDNA
atggcgctgtcctgggttcttacagtcctgagcctcctacctctgctggaagccctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 71 - DNA, 1254 bp, Artificial Sequence
  spTF-GLA cDNA
atgaggctcgccgtgggagccctgctggtctgcgccgtcctggggctgtgtctggctctggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 72 - DNA, 1254 bp, Artificial Sequence
  sp7-GLAco4 cDNA
atggcctttctgtggctgctgtcctgctgggccctgctggggaccacctttggcctggacaatggcctggccaggacccccaccatggggtggctgcactgggagaggttcatgtgcaacctggattgccaggaggagccagattcttgcatctctgagaagctgttcatggagatggctgagctgatggtgtctgagggctggaaggatgctggctatgagtacctgtgcattgatgactgctggatggctccccagagggactctgagggcagactgcaggctgacccccagaggttcccccatgggatcaggcagctggccaactatgtgcacagcaagggcctgaagctgggcatttatgctgatgtgggcaacaagacctgtgctggcttccctggctcttttggctattatgacattgatgctcagacctttgctgactggggggtggacctgctgaagtttgatgggtgctattgtgactctctggagaacctggctgatggctataagcatatgtctctggccctgaacaggactggcaggagcattgtctacagctgtgagtggcctctgtacatgtggcctttccagaagcctaactacactgagatcaggcagtattgcaaccactggaggaactttgctgacattgatgactcttggaagtctattaagagcattctggattggaccagcttcaaccaggaaaggattgtggatgtggctgggcctgggggctggaatgaccctgacatgctggtgattgggaactttggcctgagctggaaccagcaggtgacccagatggccctgtgggccatcatggctgcccccctgttcatgtccaatgacctgaggcacatcagcccccaggccaaggccctgctgcaggataaggatgtgattgccatcaatcaggaccccctggggaagcagggctaccagctgaggcagggggacaactttgaggtgtgggagagacctctgtctgggctggcctgggctgtggccatgatcaacaggcaggagattgggggccccaggagctataccattgctgtggcctctctggggaagggggtggcctgcaaccctgcctgcttcatcacccagctgctgcctgtgaagaggaagctgggcttctatgagtggaccagcaggctgaggagccacattaatcccactggcactgtgctgctgcagctggagaacaccatgcagatgtctctgaaggatctgctgtgataa

SEQ ID NO: 73 - DNA, 1251 bp, Artificial Sequence
  sp7-GLAcoBC0 cDNA
atggcctttctgtggctgctgtcctgctgggccctgctggggaccacctttggcctggacaatggcctggccagaacccccaccatgggctggctgcactgggagaggttcatgtgcaaccttgactgccaggaggagcctgacagctgcatctctgagaagctgttcatggagatggctgagctcatggtgtcagaggggtggaaggatgcaggctatgagtacctgtgcatagatgactgctggatggctccccagagggactcagagggcaggctgcaggctgaccctcagaggttcccccatggcatcaggcagctggccaactatgtgcacagcaagggcctcaagctgggaatctatgctgatgtgggcaacaaaacatgtgcaggcttcccaggctcttttggctactatgacatagatgcccagacctttgcagactggggagttgacctgctgaagtttgatgggtgctactgtgacagcctggagaacctggctgatgggtacaagcacatgtccctggccctgaacaggacaggcaggtccattgtgtacagctgtgagtggcccctgtacatgtggcccttccagaagcccaactacacagagatcagacagtactgcaaccactggagaaactttgctgacattgatgactcctggaagagcatcaagtccatcctggactggaccagcttcaaccaggagaggattgtggatgtggctggcccagggggctggaatgaccctgacatgctggtcattggcaactttggcctgagctggaaccagcaggtgacccagatggccctgtgggccatcatggctgcccccctcttcatgagcaatgacctgagacacatctccccccaagccaaggccctgttgcaggacaaggatgtgattgccatcaatcaggaccccttgggcaagcagggctaccagctgagacagggggacaactttgaggtgtgggagagacccctctctggcttggcctgggctgtggccatgatcaacagacaggagattgggggccccagaagctacaccattgctgttgccagcctgggcaagggggtggcctgcaaccctgcctgcttcatcacacagctgctgcctgtgaagaggaagctggggttctatgagtggacctctaggctgagaagtcacatcaaccccacaggcacagtgctgctgcagttggagaacaccatgcagatgagcctgaaggacctgctctga

SEQ ID NO: 74 - DNA, 1251 bp, Artificial Sequence
  sp7-GLAcoH0 cDNA
atggcctttctgtggctgctgtcctgctgggccctgctggggaccacctttggcctggacaatggtctggccaggactcccaccatgggctggctgcactgggagaggttcatgtgcaacctggactgccaggaggagccagactcctgcatcagtgagaagctcttcatggagatggctgagctcatggtctctgaagggtggaaggatgctgggtatgagtacctgtgtattgatgactgctggatggccccccaaagggactcagagggcaggctgcaggcagaccctcagaggttccctcatgggatcaggcagcttgccaactatgtccacagcaaggggctgaagctggggatctatgctgatgttgggaacaagacctgtgctggcttcccaggctcctttggctactatgatattgatgcacagacatttgcagattggggggtggatctgctgaagtttgatgggtgctactgtgactccctggagaaccttgcagatgggtacaagcacatgtccctggccctgaacaggactggcagaagcatagtctactcctgtgagtggcccctgtacatgtggcccttccagaagcccaactacacagagatcagacagtactgcaaccactggaggaactttgcagacattgatgattcctggaagagtatcaagagcatcctagactggacaagctttaaccaggagaggattgtggatgtagcaggcccagggggctggaatgacccagacatgcttgtgattggcaactttggcctcagctggaaccagcaggtgactcagatggccctgtgggccatcatggctgcccccctgttcatgtctaatgacctcaggcacatcagcccacaagccaaggcactcctccaggacaaggatgtgatagccatcaaccaggaccccttggggaagcaggggtaccagctaaggcagggggacaactttgaggtgtgggaaaggcccctctcagggcttgcctgggcagtagctatgatcaacaggcaggagattggtggccccagatcttacactattgcagtggctagcctggggaagggggtggcctgcaaccctgcctgcttcatcacacagctcctcccagtcaagaggaagttggggttctatgagtggacttccaggctgagatcccacatcaaccccacaggcactgtgctgctgcagctagagaacaccatgcagatgagcctgaaggacctcctctga

SEQ ID NO: 75 - DNA, 1251 bp, Artificial Sequence
  sp7-GLAcoH6 cDNA
atggcctttctgtggctgctgtcctgctgggccctgctggggaccacctttggcctggacaatggcttggccaggactcccaccatgggctggctgcactgggagaggttcatgtgcaacttggactgccaggaggagccagactcctgcatctctgagaagctcttcatggagatggctgagctcatggtctcagaggggtggaaggatgcagggtatgagtacctctgcattgatgactgctggatggcccctcaaagggattcagagggtaggctccaggcagacccccagaggttcccccatggcatcaggcagcttgctaactatgtccacagcaagggcctgaagctggggatctatgctgatgttggcaacaagacctgtgctggcttcccaggttcctttgggtactatgacattgatgcacagacctttgcagactggggggtagacctcctaaagtttgatgggtgctactgtgacagcttggagaacctggcagatggctacaagcacatgagccttgccctgaacaggactggcagaagcatagtgtactcctgtgagtggcccctgtacatgtggcccttccagaagcccaactacacagagatcagacagtactgcaaccactggaggaactttgctgacatagatgactcctggaagagcataaagagcatcctggactggacatccttcaaccaggagaggattgtggatgtggctgggccagggggctggaatgacccagacatgcttgtgataggcaactttggcctcagctggaaccagcaggtgactcagatggccctctgggccatcatggctgcccccctgttcatgagcaatgacctcaggcacatcagcccccaagccaaggccctcctccaggacaaggatgtgatagccatcaatcaggaccccctaggcaagcaggggtaccagctcagacagggtgacaactttgaggtgtgggaaaggcctctgtcaggccttgcctgggcagtggctatgatcaacaggcaggagattggtgggcctaggagctacaccattgcagtggcctccctggggaagggagtggcctgcaacccagcctgcttcatcacacagctcctccctgtgaagaggaagcttggcttctatgaatggacctccaggctgaggagtcacatcaaccccacagggactgtcctcctacagctagagaacaccatgcagatgtccctgaaggacctgctgtga

SEQ ID NO: 76 - DNA, 1251 bp, Artificial Sequence
  sp7-GLAvar45v2 cDNA
atggcctttctgtggctgctgtcctgctgggccctgctggggaccacctttggcctggacaatggcctggccaggacccccaccatgggctggctgcactgggagagattcatgtgcaacctggactgccaggaggagcctgacagctgcatctctgagaagctgttcatggagatggcagagctgatggtgtctgagggctggaaggatgctggctatgagtacctgtgcattgatgactgctggatggccccccagagagactctgagggcaggctgcaggcagacccccagaggttcccccatggcatcagacagctggccaactatgtgcacagcaagggcctgaagctgggcatctatgctgatgtgggcaacaagacctgcgcaggcttccctggctcctttggctactatgacattgatgcccagacctttgctgactggggagtggacctgctgaagtttgatggctgctactgtgactccctggagaacctggctgatggctacaagcacatgtccctggccctgaacaggacaggcagatccattgtgtacagctgtgagtggcccctgtacatgtggccattccagaagcccaactacacagagatcaggcagtactgcaaccactggagaaactttgcagacattgatgattcctggaagagcatcaagtccatcctggactggacctccttcaaccaggagagaattgtggatgtggcaggccctgggggctggaatgacccagacatgctggtcattggcaactttggcctgtcctggaaccagcaggtgacccagatggccctgtgggccatcatggcagcccccctgttcatgagcaatgacctgagacacatcagcccacaggccaaggccctgctgcaggacaaggatgtgattgccatcaaccaggaccctctgggcaagcagggctaccagctgaggcagggagacaactttgaggtgtgggagaggcccctgtctggcctggcctgggcagtggccatgatcaacaggcaggagattggaggccccaggagctacaccattgcagtggccagcctgggcaagggggtggcctgcaacccagcctgcttcatcacccagctgctgcctgtgaagaggaagctgggcttctatgagtggacctccagactgaggagccacatcaaccccacaggcacagtgctgctgcagctggagaacaccatgcagatgagcctgaaggacctgctgtga

SEQ ID NO: 77 - DNA, 1333 bp, Artificial Sequence
  sp7-GLA_WT_intronIgHA cDNA
atggcctttctgtggctgctgtcctgctgggccctgctggggaccacctttggcctggacaatggattggcaaggacgcctaccatgggctggctgcactgggagcgcttcatgtgcaaccttgactgccaggtgagtacaggaggtggagagtggccagcccttctcatgttcagagaacatggttaactggttaagtcatgtcgtcccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 78 - DNA, 1361 bp, Artificial Sequence
  sp7-GLA_WT_intronIgHmu cDNA
atggcctttctgtggctgctgtcctgctgggccctgctggggaccacctttggcctggacaatggattggcaaggacgcctaccatgggctggctgcactgggagcgcttcatgtgcaaccttgactgccaggtaagaaccaaaccctcccagcaggggtgcccaggcccaggcatggcccagagggagcagcgggtggggcttaggccaagctgagctcacaccttgacctttcattacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 79 - DNA, 1418 bp, Artificial Sequence
  sp7-GLA_WT_intronRBP4 cDNA
atggcctttctgtggctgctgtcctgctgggccctgctggggaccacctttggcctggacaatggattggcaaggacgcctaccatgggctggctgcactgggagcgcttcatgtgcaaccttgactgccaggtcagtggccgccggggcagctgcgccctttgcgctccagggttccccaagggccctgcctgctgatcgccacgtgggcattgtgaagggaagggagcaccgaatgggtggagggagggaggaagccctttgcccggcttggctgaggatccccttggcttttgcaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 80 - DNA, 1712 bp, Artificial Sequence
  sp7-GLA_WT_intronVTN1 cDNA
atggcctttctgtggctgctgtcctgctgggccctgctggggaccacctttggcctggacaatggattggcaaggacgcctaccatgggctggctgcactgggagcgcttcatgtgcaaccttgactgccaggtatggagagagggcaagtcttgcttctccctcaaaagggctgaaaccccttggtattggtagagccaggccggctggagggggctgtggttgtggagctatcgatcaaagtctgtttgctcaggccagactttgcttctgttgaccttttggggaaagctcagctctacctggaccccacaccttggactttgcctagcacagctgagagcacagccagcagagggaggggctgtggctgaggagtttagggggcctgggggggtggggtcgagacaccagtgatatggtggagggaaagcacagggggaagggaattggactgagagtcaaaggcctggctctgccattcgctgctgtgtgtctttgggcaaggtgcagcagatgaactctaatggccccgctggaaggggcaagattcggacccccaagacctctcattcaccccttccctgccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttga

SEQ ID NO: 81 - DNA, 1251 bp, Artificial Sequence
  sp7-GLA 7 mut cDNA
atggcctttctgtggctgctgtcctgctgggccctgctggggaccacctttggcctggacaatggattggcaaggacgcctaccatgggctggctgcactgggagcgcttcatgtgcaaccttgactgcaaagaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctgaacgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaagaacccaattatacagaaatccgacagtactgcaatcactggcgaaattttgctgacattgatgattcctggcagagtataaagagtatcttggactggacatctaccaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacaggaagacaactttgaagtgtgggaacgacctctctcaaacttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 82 - protein, 406 aa, Artificial Sequence
  sp7-GLA aa
MAFLWLLSCWALLGTTFGMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 83 - protein, 406 aa, Artificial Sequence
  spAHSG-GLA aa
MKSLVLLLCLAQLWGCHSMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 84 - protein, 405 aa, Artificial Sequence
  spCD300-GLA aa
MWLPWALLLLWVPGCFAMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 85 - protein, 419 aa, Artificial Sequence
  spGLA-GLA aa
MQLRNPELHLGCALALRFLALVSWDIPGARAMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 86 - protein, 416 aa, Artificial Sequence
  spLAMP1-GLA aa
MAAPGSARRPLLLLLLLLLLGLMHCASAMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 87 - protein, 413 aa, Artificial Sequence
  spNotch2-GLA aa
MPALRPALLWALLALWLCCAAPAHAMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 88 - protein, 406 aa, Artificial Sequence
  spORM1-GLA aa
MALSWVLTVLSLLPLLEAMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 89 - protein, 407 aa, Artificial Sequence
  spTF-GLA aa
MRLAVGALLVCAVLGLCLAMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 90 - protein, 416 aa, Artificial Sequence
  sp7-GLA 7 mut aa
MAFLWLLSCWALLGTTFGLDNGLARTPTMGWLHWERFMCNLDCKEEPDSCISEKLFMEMAELMVSEGWKDAGYEYLCIDDCWMAPQRDSEGRLQADPERFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQEPNYTEIRQYCNHWRNFADIDDSWQSIKSILDWTSTNQERIVDVAGPGGWNDPDMLVIGNFGLSWNQQVTQMALWAIMAAPLFMSNDLRHISPQAKALLQDKDVIAINQDPLGKQGYQLRQEDNFEVWERPLSNLAWAVAMINRQEIGGPRSYTIAVASLGKGVACNPACFITQLLPVKRKLGFYEWTSRLRSHINPTGTVLLQLENTMQMSLKDLL

SEQ ID NO: 91 - DNA, 3125 bp, Artificial Sequence
  pAAV_ApoE_hAAT_HBB2m1_sp7.GLAco4_BGH228
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgtcctgctgggccctgctggggaccacctttggcctggacaatggcctggccaggacccccaccatggggtggctgcactgggagaggttcatgtgcaacctggattgccaggaggagccagattcttgcatctctgagaagctgttcatggagatggctgagctgatggtgtctgagggctggaaggatgctggctatgagtacctgtgcattgatgactgctggatggctccccagagggactctgagggcagactgcaggctgacccccagaggttcccccatgggatcaggcagctggccaactatgtgcacagcaagggcctgaagctgggcatttatgctgatgtgggcaacaagacctgtgctggcttccctggctcttttggctattatgacattgatgctcagacctttgctgactggggggtggacctgctgaagtttgatgggtgctattgtgactctctggagaacctggctgatggctataagcatatgtctctggccctgaacaggactggcaggagcattgtctacagctgtgagtggcctctgtacatgtggcctttccagaagcctaactacactgagatcaggcagtattgcaaccactggaggaactttgctgacattgatgactcttggaagtctattaagagcattctggattggaccagcttcaaccaggaaaggattgtggatgtggctgggcctgggggctggaatgaccctgacatgctggtgattgggaactttggcctgagctggaaccagcaggtgacccagatggccctgtgggccatcatggctgcccccctgttcatgtccaatgacctgaggcacatcagcccccaggccaaggccctgctgcaggataaggatgtgattgccatcaatcaggaccccctggggaagcagggctaccagctgaggcagggggacaactttgaggtgtgggagagacctctgtctgggctggcctgggctgtggccatgatcaacaggcaggagattgggggccccaggagctataccattgctgtggcctctctggggaagggggtggcctgcaaccctgcctgcttcatcacccagctgctgcctgtgaagaggaagctgggcttctatgagtggaccagcaggctgaggagccacattaatcccactggcactgtgctgctgcagctggagaacaccatgcagatgtctctgaaggatctgctgtgataaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 92 - DNA, 3122 bp, Artificial Sequence
  pAAV_ApoE_hAAT_HBB2m1_sp7.GLAcoBC0_BGH228
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctgctgagctgctgggccctgctggggaccacctttgggctggacaatggcctggccagaacccccaccatgggctggctgcactgggagaggttcatgtgcaaccttgactgccaggaggagcctgacagctgcatctctgagaagctgttcatggagatggctgagctcatggtgtcagaggggtggaaggatgcaggctatgagtacctgtgcatagatgactgctggatggctccccagagggactcagagggcaggctgcaggctgaccctcagaggttcccccatggcatcaggcagctggccaactatgtgcacagcaagggcctcaagctgggaatctatgctgatgtgggcaacaaaacatgtgcaggcttcccaggctcttttggctactatgacatagatgcccagacctttgcagactggggagttgacctgctgaagtttgatgggtgctactgtgacagcctggagaacctggctgatgggtacaagcacatgtccctggccctgaacaggacaggcaggtccattgtgtacagctgtgagtggcccctgtacatgtggcccttccagaagcccaactacacagagatcagacagtactgcaaccactggagaaactttgctgacattgatgactcctggaagagcatcaagtccatcctggactggaccagcttcaaccaggagaggattgtggatgtggctggcccagggggctggaatgaccctgacatgctggtcattggcaactttggcctgagctggaaccagcaggtgacccagatggccctgtgggccatcatggctgcccccctcttcatgagcaatgacctgagacacatctccccccaagccaaggccctgttgcaggacaaggatgtgattgccatcaatcaggaccccttgggcaagcagggctaccagctgagacagggggacaactttgaggtgtgggagagacccctctctggcttggcctgggctgtggccatgatcaacagacaggagattgggggccccagaagctacaccattgctgttgccagcctgggcaagggggtggcctgcaaccctgcctgcttcatcacacagctgctgcctgtgaagaggaagctggggttctatgagtggacctctaggctgagaagtcacatcaaccccacaggcacagtgctgctgcagttggagaacaccatgcagatgagcctgaaggacctgctctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 93 - DNA, 3122 bp, Artificial Sequence
  pAAV_ApoE_hAAT_HBB2m1_sp7.GLAcoH0_BGH228
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctcctgagctgctgggccctcctggggaccacctttgggctggacaatggtctggccaggactcccaccatgggctggctgcactgggagaggttcatgtgcaacctggactgccaggaggagccagactcctgcatcagtgagaagctcttcatggagatggctgagctcatggtctctgaagggtggaaggatgctgggtatgagtacctgtgtattgatgactgctggatggccccccaaagggactcagagggcaggctgcaggcagaccctcagaggttccctcatgggatcaggcagcttgccaactatgtccacagcaaggggctgaagctggggatctatgctgatgttgggaacaagacctgtgctggcttcccaggctcctttggctactatgatattgatgcacagacatttgcagattggggggtggatctgctgaagtttgatgggtgctactgtgactccctggagaaccttgcagatgggtacaagcacatgtccctggccctgaacaggactggcagaagcatagtctactcctgtgagtggcccctgtacatgtggcccttccagaagcccaactacacagagatcagacagtactgcaaccactggaggaactttgcagacattgatgattcctggaagagtatcaagagcatcctagactggacaagctttaaccaggagaggattgtggatgtagcaggcccagggggctggaatgacccagacatgcttgtgattggcaactttggcctcagctggaaccagcaggtgactcagatggccctgtgggccatcatggctgcccccctgttcatgtctaatgacctcaggcacatcagcccacaagccaaggcactcctccaggacaaggatgtgatagccatcaaccaggaccccttggggaagcaggggtaccagctaaggcagggggacaactttgaggtgtgggaaaggcccctctcagggcttgcctgggcagtagctatgatcaacaggcaggagattggtggccccagatcttacactattgcagtggctagcctggggaagggggtggcctgcaaccctgcctgcttcatcacacagctcctcccagtcaagaggaagttggggttctatgagtggacttccaggctgagatcccacatcaaccccacaggcactgtgctgctgcagctagagaacaccatgcagatgagcctgaaggacctcctctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 94 - DNA, 3122 bp, Artificial Sequence
  pAAV_ApoE_hAAT_HBB2m1_sp7.GLAcoH6_BGH228 sequence
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctgctgagctgctgggccctgctggggaccacatttggcctggacaatggcttggccaggactcccaccatgggctggctgcactgggagaggttcatgtgcaacttggactgccaggaggagccagactcctgcatctctgagaagctcttcatggagatggctgagctcatggtctcagaggggtggaaggatgcagggtatgagtacctctgcattgatgactgctggatggcccctcaaagggattcagagggtaggctccaggcagacccccagaggttcccccatggcatcaggcagcttgctaactatgtccacagcaagggcctgaagctggggatctatgctgatgttggcaacaagacctgtgctggcttcccaggttcctttgggtactatgacattgatgcacagacctttgcagactggggggtagacctcctaaagtttgatgggtgctactgtgacagcttggagaacctggcagatggctacaagcacatgagccttgccctgaacaggactggcagaagcatagtgtactcctgtgagtggcccctgtacatgtggcccttccagaagcccaactacacagagatcagacagtactgcaaccactggaggaactttgctgacatagatgactcctggaagagcataaagagcatcctggactggacatccttcaaccaggagaggattgtggatgtggctgggccagggggctggaatgacccagacatgcttgtgataggcaactttggcctcagctggaaccagcaggtgactcagatggccctctgggccatcatggctgcccccctgttcatgagcaatgacctcaggcacatcagcccccaagccaaggccctcctccaggacaaggatgtgatagccatcaatcaggaccccctaggcaagcaggggtaccagctcagacagggtgacaactttgaggtgtgggaaaggcctctgtcaggccttgcctgggcagtggctatgatcaacaggcaggagattggtgggcctaggagctacaccattgcagtggcctccctggggaagggagtggcctgcaacccagcctgcttcatcacacagctcctccctgtgaagaggaagcttggcttctatgaatggacctccaggctgaggagtcacatcaaccccacagggactgtcctcctacagctagagaacaccatgcagatgtccctgaaggacctgctgtgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 95 - DNA, 3117 bp, Artificial Sequence
  pAAV_ApoE_hAAT_HBB2m1_sp7.GLAvar45v2_BGH228
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcagccaccatggcctttctgtggctgctgtcctgctgggccctgctggggaccacctttggcctggacaatggcctggccaggacccccaccatgggctggctgcactgggagagattcatgtgcaacctggactgccaggaggagcctgacagctgcatctctgagaagctgttcatggagatggcagagctgatggtgtctgagggctggaaggatgctggctatgagtacctgtgcattgatgactgctggatggccccccagagagactctgagggcaggctgcaggcagacccccagaggttcccccatggcatcagacagctggccaactatgtgcacagcaagggcctgaagctgggcatctatgctgatgtgggcaacaagacctgtgcaggcttccctggctcctttggctactatgacattgatgcccagacctttgctgactggggagtggacctgctgaagtttgatggctgctactgtgactccctggagaacctggctgatggctacaagcacatgtccctggccctgaacaggacaggcagatccattgtgtacagctgtgagtggcccctgtacatgtggccattccagaagcccaactacacagagatcaggcagtactgcaaccactggagaaactttgcagacattgatgattcctggaagagcatcaagtccatcctggactggacctccttcaaccaggagagaattgtggatgtggcaggccctgggggctggaatgacccagacatgctggtcattggcaactttggcctgtcctggaaccagcaggtgacccagatggccctgtgggccatcatggcagcccccctgttcatgagcaatgacctgagacacatcagcccacaggccaaggccctgctgcaggacaaggatgtgattgccatcaaccaggaccctctgggcaagcagggctaccagctgaggcagggagacaactttgaggtgtgggagaggcccctgtctggcctggcctgggcagtggccatgatcaacaggcaggagattggaggccccaggagctacaccattgcagtggccagcctgggcaagggggtggcctgcaacccagcctgcttcatcacccagctgctgcctgtgaagaggaagctgggcttctatgagtggacctccagactgaggagccacatcaaccccacaggcacagtgctgctgcagctggagaacaccatgcagatgagcctgaaggacctgctgtgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 96 - DNA, 3204 bp, Artificial Sequence
  AAV_ApoE_HAT_sp7_GLA_WT_intronIgHA
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtgagtacaggaggtggagagtggccagcccttctcatgttcagagaacatggttaactggttaagtcatgtcgtcccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 97 - DNA, 3232 bp, Artificial Sequence
  AAV_ApoE_HAT_sp7_GLA_WT_intronIgHmu
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtaagaaccaaaccctcccagcaggggtgcccaggcccaggcatggcccagagggagcagcgggtggggcttaggccaagctgagctcacaccttgacctttcattacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 98 - DNA, 3289 bp, Artificial Sequence
  AAV_ApoE_HAT_sp7_GLA_WT_intronRBP4
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtcagtggccgccggggcagctgcgccctttgcgctccagggttccccaagggccctgcctgctgatcgccacgtgggcattgtgaagggaagggagcaccgaatgggtggagggagggaggaagccctttgcccggcttggctgaggatccccttggcttttgcaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 99 - DNA, 3583 bp, Artificial Sequence
  AAV_ApoE_HAT_sp7_GLA_WT_intronVTN1
cctgcaggcagctgcgcgctcgctcgctcactgaggccgcccgggcaaagcccgggcggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacaggtcctgggcaacctgctggtctctctgctggcccatcactttggcaaagcacgcgtgccaccatggcctttctgtggctgctgtcctgctgggccctgctggggaccacctttggcctggacaatggattggcaaggacgcctaccatgggctggctgcactgggagcgcttcatgtgcaaccttgactgccaggtatggagagagggcaagtcttgcttctccctcaaaagggctgaaaccccttggtattggtagagccaggccggctggagggggctgtggttgtggagctatcgatcaaagtctgtttgctcaggccagactttgcttctgttgaccttttggggaaagctcagctctacctggaccccacaccttggactttgcctagcacagctgagagcacagccagcagagggaggggctgtggctgaggagtttagggggcctgggggggtggggtcgagacaccagtgatatggtggagggaaagcacagggggaagggaattggactgagagtcaaaggcctggctctgccattcgctgctgtgtgtctttgggcaaggtgcagcagatgaactctaatggccccgctggaaggggcaagattcggacccccaagacctctcattcaccccttccctgccacaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagttgatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 100 - protein, 398 aa, Artificial Sequence
  GLA
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 101 - protein, 416 aa, Artificial Sequence
  sp7-GLA
MAFLWLLSCWALLGTTFGLDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 102 - protein, 416 aa, Artificial Sequence
  spAHSG-GLA
MKSLVLLLCLAQLWGCHSLDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 103 - protein, 415 aa, Artificial Sequence
  spCD300-GLA
MWLPWALLLLWVPGCFALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 104 - protein, 429 aa, Artificial Sequence; identical to SEQ ID NO: 12
  spGLA-GLA

SEQ ID NO: 105 - protein, 426 aa, Artificial Sequence
  spLAMP1-GLA aa
MAAPGSARRPLLLLLLLLLLGLMHCASALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 106 - protein, 423 aa, Artificial Sequence
  spNotch2-GLA
MPALRPALLWALLALWLCCAAPAHALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 107 - protein, 416 aa, Artificial Sequence
  spORM1-GLA
MALSWVLTVLSLLPLLEALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 108 - protein, 417 aa, Artificial Sequence
  spTF-GLA
MRLAVGALLVCAVLGLCLALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 109 - protein, 426 aa, Artificial Sequence
  sp7-GLA 7 mut
MAFLWLLSCWALLGTTFGLDNGLARTPTLDNGLARTPTMGWLHWERFMCNLDCKEEPDSCISEKLFMEMAELMVSEGWKDAGYEYLCIDDCWMAPQRDSEGRLQADPERFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQEPNYTEIRQYCNHWRNFADIDDSWQSIKSILDWTSTNQERIVDVAGPGGWNDPDMLVIGNFGLSWNQQVTQMALWAIMAAPLFMSNDLRHISPQAKALLQDKDVIAINQDPLGKQGYQLRQEDNFEVWERPLSNLAWAVAMINRQEIGGPRSYTIAVASLGKGVACNPACFITQLLPVKRKLGFYEWTSRLRSHINPTGTVLLQLENTMQMSLKDLL

SEQ ID NO: 110 - protein, 738 aa, Artificial Sequence
  VP1
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL